SEQUENCE LISTING (38 sequences)

SEQ ID NO: 1 - DNA, 291 bp, Homo sapiens
atgctgacgctcggtgaacagttgcctttggtcacaagatttagaagacacagtgtccatcctcccagattggatctctttttcatatggatcttctgtttctatgtctttttaaaaaataactttttgggaaaccttttggattacaactgttcatcctcacctatgcaaagaaagggaagctattgctgggattttgaggagatggtcctagaacaattggagattcatacgcacacaaagaacctcaacccttacctcacaccagacacaaaagctaccttcaaataa

SEQ ID NO: 2 - DNA, 231 bp, Homo sapiens
atgctgacgctcggtgaacagttgcctttggtcacaagatttagaagacacagtgtccatcctcccagattggatctctttttcatatggatcttctgtttctatgtctttttaaaaaataactttttgggaaaccttttggattacaactgttcatcctcacctatgcaaagaaagggaagctattgctgggattttgaggaggcagtccgttgccactgggcagtatag

SEQ ID NO: 3 - DNA, 1962 bp, Homo sapiens
atggagccttcgggcagtgaacagttatttgaggaccctgatcctggaggcaaatcccaagatgcagaggccagaaagcagacagaatcagaacaaaaattgtctaaaatgacccacaatgctttggagaacattaacgtgattggccaaggcttgaagcatctcttccagcaccagcgcaggaggtcatcagtgtctccacatgatgtgcagcaaattcaggcagatccagaacctgaaatggatctggaaagccagaacgcatgtgctgagattgatggtgtccccacccaccccacagctctgaatcgtgtcctgcagcagattcgagtgccacccaagatgaagagagggacaagcttgcatagtaggcggggcaagccagaggccccaaagggaagtccccaaatcaacaggaagtctggtcaggagatgacagctgttatgcagtcaggccgacccaggtcttcatccacaactgatgcacctaccggctctgctatgatggaaatagcttgtgctgctgctgctgctgctgctgcatgtctaccaggagaggagggaactgcggagcggatcgaacggttggaagtaagcagccttgcccaaacatccagtgcagtggcctccagtaccgatggcagcatccacacagactctgtggatggaacaccagaccctcagcgcacaaaggctgccattgctcacctgcagcagaagatcctgaagctcacagaacaaatcaagattgcacaaacagcccgggacgacaacgttgctgaatacttgaagcttgccaacagtgcagacaaacagcaggctgcccgcatcaagcaagtctttgagaagaagaaccagaaatctgcccaaactatcctccagctgcaaaagaaacttgagcactaccacaggaagctcagagaggtagagcagaatgggatcccccggcagccaaaggatgtcttcagggacatgcaccagggtctgaaggatgtaggagcaaaggtgactggcttcagtgaaggtgtggtggatagtgtcaaaggtgggttttccagcttctcccaggccacccattcagcagcaggcgctgtagtctcaaagcccagagagattgcctcactcattcggaacaaatttggcagtgcagacaacatccccaacctgaaggactctttagaggaagggcaagtggatgatgcggggaaggctttgggagtgatttcaaactttcagtctagcccaaaatatggtagtgaagaagattgttctagtgccacttcaggctcagtgggagccaacagcaccacagggggcatcgctgtaggagcatccagctccaaaacaaacaccctggacatgcagagctcaggatttgatgcactactacatgagatccaggagatccgggaaacccaggccagactagaggaatcctttgagactctcaaggaacattatcagagggactattccttaataatgcagaccttacaggaggagcgatatagatgtgaacgattggaagaacagctaaatgacctaacagagctccaccagaatgaaatcttgaacttgaagcaggaactggcaagcatggaagaaaaaatcgcgtatcagtcctatgaacgggcccgggacatccaggaggccctggaggcatgccagacgcgcatctccaagatggagctgcagcagcagcagcagcaggtggtgcagctagaagggctggagaatgccactgcccggaaccttctgggcaaactcatcaacatcctcctggctgtcatggcagtccttttggtctttgtctccactgtagccaactgtgtggtccccctcatgaagactcgcaacaggacgttcagcactttattccttgtggtttttattgcctttctctggaagcactgggacgccctcttcagctatgtggaacggttcttttcatcccctagatga

SEQ ID NO: 4 - DNA, 366 bp, Homo sapiens
atggaagaaaaaatcgcgtatcagtcctatgaacgggcccgggacatccaggaggccctggaggcatgccagacgcgcatctccaagatggagctgcagcagcagcagcagcaggtggtgcagctagaagggctggagaatgccactgcccggaaccttctgggcaaactcatcaacatcctcctggctgtcatggcagtccttttggtctttgtctccactgtagccaactgtgtggtccccctcatgaagactcgcaacaggacgttcagcactttattccttgtggtttttattgcctttctctggaagcactgggacgccctcttcagctatgtggaacggttcttttcatcccctagatga

SEQ ID NO: 5 - DNA, 645 bp, Homo sapiens
atggagccttcgggcagtgaacagttatttgaggaccctgatcctggaggcaaatcccaagatgcagaggccagaaagcagacagaatcagaacaaaaattgtctaaaatgacccacaatgctttggagaacattaacgtgattggccaaggcttgaagcatctcttccagcaccagcgcaggaggtcatcagtgtctccacatgatgtgcagcaaattcaggcagatccagaacctgaaatggatctggaaagccagaacgcatgtgctgagattgatggtgtccccacccaccccacagctctgaatcgtgtcctgcagcagattcgagtgccacccaagatgaagagagggacaagcttgcatagtaggcggggcaagccagaggccccaaagggaagtccccaaatcaacaggaagtctggtcaggagatgacagctgttatgcagtcaggccgacccaggtcttcatccacaactgatgcacctaccagctctgctatgatggaaatagcttgtgctgctgctgctgctgctgctgcatgtctaccaggagaaatgccactgcccggaaccttctgggcaaactcatcaacatcctcctggctgtcatggcagtccttttggtctttgtctccactgtag

SEQ ID NO: 6 - DNA, 1962 bp, Homo sapiens; identical to SEQ ID NO: 3

SEQ ID NO: 7 - protein, 96 aa, Homo sapiens
MLTLGEQLPLVTRFRRHSVHPPRLDLFFIWIFCFYVFLKNNFLGNLLDYNCSSSPMQRKGSYCWDFEEMVLEQLEIHTHTKNLNPYLTPDTKATFK

SEQ ID NO: 8 - protein, 76 aa, Homo sapiens
MLTLGEQLPLVTRFRRHSVHPPRLDLFFIWIFCFYVFLKNNFLGNLLDYNCSSSPMQRKGSYCWDFEEAVRCHWAV

SEQ ID NO: 9 - protein, 653 aa, Homo sapiens
MEPSGSEQLFEDPDPGGKSQDAEARKQTESEQKLSKMTHNALENINVIGQGLKHLFQHQRRRSSVSPHDVQQIQADPEPEMDLESQNACAEIDGVPTHPTALNRVLQQIRVPPKMKRGTSLHSRRGKPEAPKGSPQINRKSGQEMTAVMQSGRPRSSSTTDAPTGSAMMEIACAAAAAAAACLPGEEGTAERIERLEVSSLAQTSSAVASSTDGSIHTDSVDGTPDPQRTKAAIAHLQQKILKLTEQIKIAQTARDDNVAEYLKLANSADKQQAARIKQVFEKKNQKSAQTILQLQKKLEHYHRKLREVEQNGIPRQPKDVFRDMHQGLKDVGAKVTGFSEGVVDSVKGGFSSFSQATHSAAGAVVSKPREIASLIRNKFGSADNIPNLKDSLEEGQVDDAGKALGVISNFQSSPKYGSEEDCSSATSGSVGANSTTGGIAVGASSSKTNTLDMQSSGFDALLHEIQEIRETQARLEESFETLKEHYQRDYSLIMQTLQEERYRCERLEEQLNDLTELHQNEILNLKQELASMEEKIAYQSYERARDIQEALEACQTRISKMELQQQQQQVVQLEGLENATARNLLGKLINILLAVMAVLLVFVSTVANCVVPLMKTRNRTFSTLFLVVFIAFLWKHWDALFSYVERFFSSPR

SEQ ID NO: 10 - protein, 26 aa, Homo sapiens
MLTLGEQLPLVTRFRRHSVHPPRLDL

SEQ ID NO: 11 - protein, 47 aa, Homo sapiens
NCSSSPMQRKGSYCWDFEEMVLEQLEIHTHTKNLNPYLTPDTKATFK

SEQ ID NO: 12 - protein, 26 aa, Homo sapiens
MLTLGEQLPLVTRFRRHSVHPPRLDL

SEQ ID NO: 13 - protein, 27 aa, Homo sapiens
NCSSSPMQRKGSYCWDFEEAVRCHWAV

SEQ ID NO: 14 - protein, 591 aa, Homo sapiens
MEPSGSEQLFEDPDPGGKSQDAEARKQTESEQKLSKMTHNALENINVIGQGLKHLFQHQRRRSSVSPHDVQQIQADPEPEMDLESQNACAEIDGVPTHPTALNRVLQQIRVPPKMKRGTSLHSRRGKPEAPKGSPQINRKSGQEMTAVMQSGRPRSSSTTDAPTGSAMMEIACAAAAAAAACLPGEEGTAERIERLEVSSLAQTSSAVASSTDGSIHTDSVDGTPDPQRTKAAIAHLQQKILKLTEQIKIAQTARDDNVAEYLKLANSADKQQAARIKQVFEKKNQKSAQTILQLQKKLEHYHRKLREVEQNGIPRQPKDVFRDMHQGLKDVGAKVTGFSEGVVDSVKGGFSSFSQATHSAAGAVVSKPREIASLIRNKFGSADNIPNLKDSLEEGQVDDAGKALGVISNFQSSPKYGSEEDCSSATSGSVGANSTTGGIAVGASSSKTNTLDMQSSGFDALLHEIQEIRETQARLEESFETLKEHYQRDYSLIMQTLQEERYRCERLEEQLNDLTELHQNEILNLKQELASMEEKIAYQSYERARDIQEALEACQTRISKMELQQQQQQVVQLEGLENATARNLLGKLIN

SEQ ID NO: 15 - protein, 6 aa, Homo sapiens
MKTRNR

SEQ ID NO: 16 - protein, 15 aa, Homo sapiens
DALFSYVERFFSSPR

SEQ ID NO: 17 - protein, 121 aa, Homo sapiens
MEEKIAYQSYERARDIQEALEACQTRISKMELQQQQQQVVQLEGLENATARNLLGKLINILLAVMAVLLVFVSTVANCVVPLMKTRNRTFSTLFLVVFIAFLWKHWDALFSYVERFFSSPR

SEQ ID NO: 18 - protein, 214 aa, Homo sapiens
MEPSGSEQLFEDPDPGGKSQDAEARKQTESEQKLSKMTHNALENINVIGQGLKHLFQHQRRRSSVSPHDVQQIQADPEPEMDLESQNACAEIDGVPTHPTALNRVLQQIRVPPKMKRGTSLHSRRGKPEAPKGSPQINRKSGQEMTAVMQSGRPRSSSTTDAPTSSAMMEIACAAAAAAAACLPGEMPLPGTFWANSSTSSWLSWQSFWSLSPL

SEQ ID NO: 19 - protein, 653 aa, Homo sapiens; identical to SEQ ID NO: 9

SEQ ID NO: 20 - protein, 56 aa, Homo sapiens
MEEKIAYQSYERARDIQEALEACQTRISKMELQQQQQQVVQLEGLENATARNLLGK

SEQ ID NO: 21 - protein, 14 aa, Homo sapiens
NCVVPLMKTRNRTF

SEQ ID NO: 22 - protein, 8 aa, Homo sapiens
ERFFSSPR

SEQ ID NO: 23 - protein, 23 aa, Homo sapiens
ILLAVMAVLLVFVSTVANCVVPL

SEQ ID NO: 24 - protein, 18 aa, Homo sapiens
TFSTLFLVVFIAFLWKHW

SEQ ID NO: 25 - protein, 59 aa, Homo sapiens
MEEKIAYQSYERARDIQEALEACQTRISKMELQQQQQQVVQLEGLENATARNLLGKLIN

SEQ ID NO: 26 - protein, 6 aa, Homo sapiens
MKTRNR

SEQ ID NO: 27 - protein, 15 aa, Homo sapiens
DALFSYVERFFSSPR

SEQ ID NO: 28 - protein, 591 aa, Homo sapiens; identical to SEQ ID NO: 14

SEQ ID NO: 29 - protein, 6 aa, Homo sapiens
MKTRNR

SEQ ID NO: 30 - protein, 15 aa, Homo sapiens
DALFSYVERFFSSPR

SEQ ID NO: 31 - DNA, 806 bp, Homo sapiens
tagaaccatggggtggggacagtcgtgagggagcgtggcctggcggcgcagcggacgcgggcctggcctcccgctcgcggcctgtcggggctgggacctgccgtcgcccccgttcgaggttgaagccccgggcctaggactcgacccccagcatcccacggggcctctttcctttcccggctcattccgctgtcattttgacctggggttcccctccagcccctcgcctcgttcccttcccagcatcccagggccgaggtattaatctctggagaagacacatccacagttagcactttcttcagatgctgacgctcggtgaacagttgcctttggtcacaagatttagaagacacagtgtccatcctcccagattggatctctttttcatatggatcttctgtttctatgtctttttaaaaaataactttttgggaaaccttttggattacaactgttcatcctcacctatgcaaagaaagggaagctattgctgggattttgaggagatggtcctagaacaattggagattcatacgcacacaaagaacctcaacccttacctcacaccagacacaaaagctaccttcaaataaatcataggcctaacttgaagagctaaaaccatgcaactccagaaagtttttgtcagaaagaaaatacaggagaaaatcttagtgaccttggggtaggcaaagatttcttaagacacaaaaagcatgaagtataaaggggaaaaaatcgctaaattggatttcatccaagttaaaaacttttaatctttgaaagatacctttaagaaaatg

SEQ ID NO: 32 - DNA, 1877 bp, Homo sapiens
ggtggttgtgggggatggaccgggtgcggcggccgcagtggaaggagcaggcgcttgagctcgagcgacggcgctggcggagacgccggctgctcctcccctccccgccggtattaatctctggagaagacacatccacagttagcactttcttcagatgctgacgctcggtgaacagttgcctttggtcacaagatttagaagacacagtgtccatcctcccagattggatctctttttcatatggatcttctgtttctatgtctttttaaaaaataactttttgggaaaccttttggattacaactgttcatcctcacctatgcaaagaaagggaagctattgctgggattttgaggaggcagtccgttgccactgggcagtatagatatgttgtccagcctgccatcactggactgtagttaaggtggatgtcttgtccagtctgctgccactgggctgtatgtaaggaggttcttctgtccaccccaccgctactggactctcttccctgtagatggtcctagaacaattggagattcatacgcacacaaagaacctcaacccttacctcacaccagacacaaaagctaccttcaaataaatcataggcctaacttgaagagctaaaaccatgcaactccagaaagtttttgtcagaaagaaaatacaggagaaaatcttagtgaccttggggtaggcaaagatttcttaagacacaaaaagcatgaagtataaaggggaaaaaatcgctaaattggatttcatccaagttaaaaacttttaatctttgaaagatacctttaagaaaatgaaaaagtacgccttgggctgggagaaaatatttgcagaacgtgtgtctgacagaggatgtgtatctagaagatataaagaattgtaactcaagaattgaaagacaaccccataagaaaagggggaaacaatttgaataaagttcatcaaagaatataaatggcaaataagcacatgaaaagatgcccaaagtcgtaagtcattagggaaatataaatttaaaccataatgagataccactgcatactccctagaatggctgtaatgaataggattagtcacatggtgacaagaatggaggatcatctggaactctcatacactgacgataggaatgtgaaatggatcaactactttggaagacaattgggcagtttctttcaaagtaaatgtgaagatgccatacgattcatccattccatttctaattattcaagagaaatgaaactgtatatccacaaaaaagacttgtacacaaacattcacagcagctattatttattggtaatagctaaaaactgtaaacagctcccatatccatcaagtgtatggataaacaaatttggtgtatttatacaatggaatactactcggcaataaaaagaacagttgatactctcaacaacctagatggacctcaaaataattcggtttaatgaatgaagccaaacttaagaagagtacattgtatgtacttggagaactaacttcttgcaatagatttttaagcactattaggagcatatgacttaaacagtttttaaaagtcagggagtaagtatgcttaaataaaatacaatctgtgaaacaaatctctgaattattatcacttcactggacactctaacttgaccatatttctgactttaatgtaactcactcttattccgtagtcacatgtttgcttgctcattggttcacattacatttattcagcatctgcttgagccaaggcactgtaactacatgttttttttagttacctacttttgtaaggtcctgtttctttggctacatctgattacagtaaacataggaagtttaataaaacaattttcatgacc

SEQ ID NO: 33 - DNA, 2801 bp, Homo sapiens
gacacagtgtccatcctcccagattggatctctttttcatatggatcttctgtttctatgtctttttaaaaaataactttttgggaaaccttttggattacaactgttcatcctcacctatgcaaagaaagggaagctattgctgggattttgaggagcttttcctaaaaggattgtacaccttagaagtgcttaaggaagagtgatgaagataggcatgaagccttcgtctcacagctgcatgcgtagtcactgttgaagcaaatgcctacctaatttgacactcttggtgtgtttaaaaaatttttttgagtttgcaaataagcatattaagtctactgatggagccttcgggcagtgaacagttatttgaggaccctgatcctggaggcaaatcccaagatgcagaggccagaaagcagacagaatcagaacaaaaattgtctaaaatgacccacaatgctttggagaacattaacgtgattggccaaggcttgaagcatctcttccagcaccagcgcaggaggtcatcagtgtctccacatgatgtgcagcaaattcaggcagatccagaacctgaaatggatctggaaagccagaacgcatgtgctgagattgatggtgtccccacccaccccacagctctgaatcgtgtcctgcagcagattcgagtgccacccaagatgaagagagggacaagcttgcatagtaggcggggcaagccagaggccccaaagggaagtccccaaatcaacaggaagtctggtcaggagatgacagctgttatgcagtcaggccgacccaggtcttcatccacaactgatgcacctaccggctctgctatgatggaaatagcttgtgctgctgctgctgctgctgctgcatgtctaccaggagaggagggaactgcggagcggatcgaacggttggaagtaagcagccttgcccaaacatccagtgcagtggcctccagtaccgatggcagcatccacacagactctgtggatggaacaccagaccctcagcgcacaaaggctgccattgctcacctgcagcagaagatcctgaagctcacagaacaaatcaagattgcacaaacagcccgggacgacaacgttgctgaatacttgaagcttgccaacagtgcagacaaacagcaggctgcccgcatcaagcaagtctttgagaagaagaaccagaaatctgcccaaactatcctccagctgcaaaagaaacttgagcactaccacaggaagctcagagaggtagagcagaatgggatcccccggcagccaaaggatgtcttcagggacatgcaccagggtctgaaggatgtaggagcaaaggtgactggcttcagtgaaggtgtggtggatagtgtcaaaggtgggttttccagcttctcccaggccacccattcagcagcaggcgctgtagtctcaaagcccagagagattgcctcactcattcggaacaaatttggcagtgcagacaacatccccaacctgaaggactctttagaggaagggcaagtggatgatgcggggaaggctttgggagtgatttcaaactttcagtctagcccaaaatatggtagtgaagaagattgttctagtgccacttcaggctcagtgggagccaacagcaccacagggggcatcgctgtaggagcatccagctccaaaacaaacaccctggacatgcagagctcaggatttgatgcactactacatgagatccaggagatccgggaaacccaggccagactagaggaatcctttgagactctcaaggaacattatcagagggactattccttaataatgcagaccttacaggaggagcgatatagatgtgaacgattggaagaacagctaaatgacctaacagagctccaccagaatgaaatcttgaacttgaagcaggaactggcaagcatggaagaaaaaatcgcgtatcagtcctatgaacgggcccgggacatccaggaggccctggaggcatgccagacgcgcatctccaagatggagctgcagcagcagcagcagcaggtggtgcagctagaagggctggagaatgccactgcccggaaccttctgggcaaactcatcaacatcctcctggctgtcatggcagtccttttggtctttgtctccactgtagccaactgtgtggtccccctcatgaagactcgcaacaggacgttcagcactttattccttgtggtttttattgcctttctctggaagcactgggacgccctcttcagctatgtggaacggttcttttcatcccctagatgatgctggcacagaaggcattgttccctaccctctggcgagtgcatgcagcagagagttagacagcaacttacctactctgaagttttctacaacaaaaaaagagttgagtgaatctgtttacatttagaataatgtttttttcttcaagagacgcaattgcaatagtattttttagattttatccaagaagttttttgggcgaaaatcttggatcatttttatgtagcatgattttccttgggatgcaaatcttaaaacagtcctttaatatgaaccaacaatctggagcacaccgaagggcaatctaaattgtggcttgaaggactgcactaaaacccactaaaaagatgcgaaaacctgatgagggcaaaccagttaaacctaacaccctgccttgtctgggctcatcacctctccctatcccagactaactttactgtgaaatcctaccacattccatgtctgaatttttggattcggggtggattttcgttgtcc

SEQ ID NO: 34 - DNA, 512 bp, Homo sapiens
aaaactgtaaacagctcccatatccatcaagtgtatggataaacaaatttggtgtatttatacaatggaatactactcggcaataaaaagaacagttgatactctcaacaacctagatggacctcaaaataattcggtttaatgaatgaagccaaacttaagaagagtacattgtatgtacttggagaactaacttcttgcaatagatttttaagcactattaggagcatatgacttaaacagtttttaaaagtcagggagtaagtatgcttaaataaaatacaatctgtgaaacaaatctctgaattattatcacttcactggacactctaacttgaccatatttctgactttaatgtaactcactcttattccgtagtcacatgtttgcttgctcattggttcacattacatttattcagcatctgcttgagccaaggcactgtaactacatgttttttttagttacctacttttgtaaggtcctgtttctttggctacatctgattaca

SEQ ID NO: 35 - DNA, 1447 bp, Homo sapiens
gaaggcagtatagagactcctatcaaggataaagaagaactaaatagagtttagtaacatcgtaaaaaggatgtgaacgattggaagaacagctaaatgacctaacagagctccaccagaatgaaatcttgaacttgaagcaggaactggcaagcatggaagaaaaaatcgcgtatcagtcctatgaacgggcccgggacatccaggaggccctggaggcatgccagacgcgcatctccaagatggagctgcagcagcagcagcagcaggtggtgcagctagaagggctggagaatgccactgcccggaaccttctgggcaaactcatcaacatcctcctggctgtcatggcagtccttttggtctttgtctccactgtagccaactgtgtggtccccctcatgaagactcgcaacaggacgttcagcactttattccttgtggtttttattgcctttctctggaagcactgggacgccctcttcagctatgtggaacggttcttttcatcccctagatgatgctggcacagaaggcattgttccctaccctctggcgagtgcatgcagcagagagttagacagcaacttacctactctgaagttttctacaacaaaaaaagagttgagtgaatctgtttacatttagaataatgtttttttcttcaagagacgcaattgcaatagtattttttagattttatccaagaagttttttgggcgaaaatcttggatcatttttatgtagcatgattttccttgggatgcaaatcttaaaacagtcctttaatatgaaccaacaatctggagcacaccgaagggcaatctaaattgtggcttgaaggactgcactaaaacccactaaaaagatgcgaaaacctgatgagggcaaaccagttaaacctaacaccctgccttgtctgggctcatcacctctccctatcccagactaactttactgtgaaatcctaccacattccatgtctgaatttttggattcggggtggattttcgttgtccgtggaagaacacatggatctctctggctttctcacccaagttggccacttacgctaatcctggaagtatgatcacttttgaacctgccccttaaccttgacgaggatacaaaagtgaaagcatcatcccccaaaggatcactgcacagtcctactacagtatttttaagtagccctctaaatacttaattttaagcaaaatcccttggccgcacttttaaggtttttttatatgtgtatagttaccaacctaaaaataaaaaatccgaacagcatacttgaagaatgtaatactcaaactctcagtgcttccttatggtttctaataggattttttattattgttattattattattgggtttttttggacagggttgggagggtcttttatttttcctttgaaataaagaagtgatgtttttaaatg

SEQ ID NO: 36 - DNA, 1584 bp, Homo sapiens
cgggtgcggcggccgcagtggaaggagcaggcgcttgagctcgagcgacggcgctggcggagacgccggctgctcctcccctccccgccggtattaatctctggagaagacacatccacagttagcactttcttcagatgctgacgctcggtgaacagttgcctttggtcacaagatttagaagacacagtgtccatcctcccagattggatctctttttcatatggatcttctgtttctatgtctttttaaaaaataactttttgggaaaccttttggattacaactgttcatcctcacctatgcaaagaaagggaagctattgctgggattttgaggagcttttcctaaaaggattgtacaccttagaagtgcttaaggaagagtgatgaagataggcatgaagccttcgtctcacagctgcatgcgtagtcactgttgaagcaaatgcctacctaatttgacactcttggtgtgtttaaaaaatttttttgagtttgcaaataagcatattaagtctactgatggagccttcgggcagtgaacagttatttgaggaccctgatcctggaggcaaatcccaagatgcagaggccagaaagcagacagaatcagaacaaaaattgtctaaaatgacccacaatgctttggagaacattaacgtgattggccaaggcttgaagcatctcttccagcaccagcgcaggaggtcatcagtgtctccacatgatgtgcagcaaattcaggcagatccagaacctgaaatggatctggaaagccagaacgcatgtgctgagattgatggtgtccccacccaccccacagctctgaatcgtgtcctgcagcagattcgagtgccacccaagatgaagagagggacaagcttgcatagtaggcggggcaagccagaggccccaaagggaagtccccaaatcaacaggaagtctggtcaggagatgacagctgttatgcagtcaggccgacccaggtcttcatccacaactgatgcacctaccagctctgctatgatggaaatagcttgtgctgctgctgctgctgctgctgcatgtctaccaggagaaatgccactgcccggaaccttctgggcaaactcatcaacatcctcctggctgtcatggcagtccttttggtctttgtctccactgtagccaactgtgtggtccccctcatgaagactcgcaacaggacgttcagcactttattccttgtggtttttattgcctttctctggaagcactgggacgccctcttcagctatgtggaacggttcttttcatcccctagatgatgctggcacagaaggcattgttccctaccctctggcgagtgcatgcagcagagagttagacagcaacttacctactctgaagttttctacaacaaaaaaagagttgagtgaatctgtttacatttagaataatgtttttttcttcaagagacgcaattgcaatagtattttttagattttatccaagaagttttttgggcgaaaatcttggatcatttttatgtagcatgattttccttgggatgcaaatcttaaaacagtcctttaatatgaac

SEQ ID NO: 37 - DNA, 2801 bp, Homo sapiens; identical to SEQ ID NO: 33

SEQ ID NO: 38 - DNA, 453 bp, Homo sapiens
  misc_feature (94)..(94): n is a, c, g, or t
  misc_feature (201)..(201): n is a, c, g, or t
  misc_feature (211)..(214): n is a, c, g, or t
gatgagggcaaaccagttaaacctaacaccctgccttgtctgggctcatcacctctccctatcccagactaactttactgtgaaatcctaccancattccatgtctgaatttttggattcggggtggattttcgttgtccgtggaagaacacatggatctctctggctttctcacccaagttggccacttacgctaatccnggaagtatgnnnncttttgaacctgccccttaaccttgacgaggatacaaaagtgaaagcatcatcccccaaaggatcactgcacagtcctactacagtatttttaagtagccctctaaatacttaattttaagcaaaatcccttggccgcacttttaaggtttttttatatgtgtatagttaccaacctaaaaataaaaaatccgaacagcatacttgaagaatgtaatactcaaactctcagtgcttcct

(19) United States
(12) Patent Application Publication
(10) Pub. No.: US 2007/0274988 A1
(43) Pub. Date: Nov. 29, 2007

(54) Kiaa0779, Splice Variants Thereof, and Methods of Their Use

(75) Inventors: Justin G.P. Wong, Oakland, CA (US); Kevin Hestir, Kensington, CA (US); Amy L. Tsui Collins, Oakland, CA (US); Ernestine Lee, Kensington, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: FIVE PRIME THERAPEAUTICS, INC., San Francisco, CA (US)

(21) Appl. No.: 10/575,265

(22) PCT Filed: Oct. 12, 2004
(86) PCT No.: PCT/US04/33408

§ 371 Date: May 2, 2007

Related U.S. Application Data
(60) Provisional application No. 60/510,612, filed on Oct. 10, 2003.

(51) Int. Cl. A61K 39/395; A01K 67/00; A61K 31/7088; C07H 21/00; C07K 16/00; C12N 15/63; C12N 7/01; C12P 21/08; G01N 33/50; G01N 33/53; C12Q 1/68; C12P 21/04; C12N 5/10; C12N 1/21; C07K 14/00; A61P 35/00; A61K 38/12

(52) U.S. Cl. 424/135.1; 435/235.1; 435/252.3; 435/320.1; 435/348; 435/440; 435/6; 435/69.1; 435/7.8; 435/70.3; 436/86; 514/12; 514/44; 530/324; 530/350; 530/387.3; 536/23.1; 800/4; 800/8

(57) ABSTRACT

The invention provides novel polynucleotides, related polypeptides related nucleic acid and polypeptide compositions, and related modulators, such as antibodies and small molecule modulators. The invention also provides methods to make and use these polynucleotides, polypeptides, related compositions, and modulators. These methods include diagnostic, prophylactic, and therapeutic applications. The compositions and methods of the invention are useful in treating proliferative disorders, e.g., cancers, such as kidney and pancreatic cancer; and inflammatory, immune, and viral disorders.

### PRIORITY CLAIM

This application claims the benefit of U.S. Provisional Application 60/510,612, filed Oct. 10, 2003, the disclosure of which is incorporated in its entirety.

### TECHNICAL FIELD

The present invention is related generally to novel polynucleotides and novel polypeptides encoded thereby, their compositions, antibodies directed thereto, and other agonists or antagonists thereto. The polynucleotides and polypeptides are useful in diagnostic, prophylactic, and therapeutic applications for a variety of diseases, disorders, syndromes and conditions, as well as in discovering new diagnostics, prophylactics, and therapeutics for such diseases, disorders, syndromes, and conditions (hereinafter disorders). The present invention also relates to methods of modulating biological activities through the use of the novel polynucleotides and novel polypeptides of the invention and through the use of agonists and antagonists, such as antibodies and small molecule drugs directed thereto.

This application further relates to the field of polypeptides that are associated with regulating cell growth and differentiation of cells that are over-expressed in cancer, such as in malignant kidney cancer, and pancreatic cancer, and/or that can be associated with proliferation or inhibition of cancer growth. These polypeptides may also be associated with other conditions, such as inflammatory, immune, and metabolic disorders, as well as microbial infections, including viral, bacterial, fungal, and parasitic diseases, disorders, syndromes, or conditions.

This application further relates to modulators of biological activity that can specifically bind to these polynucleotides or polypeptides, or otherwise specifically modulate their activity. For example, they can directly or indirectly induce antibody-dependent cellular cytotoxicity (ADCC), complement-dependent cytotoxicity (CDC), endocytosis, apoptosis, or recruitment of other cells to effect cell activation, cell inactivation, cell growth or differentiation or inhibition thereof, and cell killing.

### BACKGROUND

Polypeptides and polynucleotides of the invention are transmembrane proteins, i.e., proteins that extend into or through the cell membrane's lipid bilayer. They can span the membrane once, or more than once. Transmembrane proteins that span the membrane once are “single transmembrane proteins” (STM), and transmembrane proteins that span the membrane more than once are “multiple transmembrane proteins” (MTM).

A single transmembrane protein typically has one transmembrane (TM) domain, spanning a series of consecutive amino acid residues, numbered on the basis of distance from the N-terminus, with the first amino aid residue at the N-terminus as number 1. A multi-transmembrane protein typically has more than one TM domain, each spanning a series of consecutive amino acid residues, numbered in the same way as the STM protein.

Transmembrane proteins, having part of their molecules on either side of the bilayers, have many and widely variant biological functions. For example, they transport molecules, e.g., ions or proteins across membranes, transduce signals across membranes, act as receptors, and function as antigens. Transmembrane proteins are often involved in cell signaling events; they can comprise signaling molecules, or can interact with signaling molecules. Abnormalities of transmembrane proteins, such as over-expression or mutation of such, are associated with human cancers. For example, tumor cells are known to use transmembrane rotors in transduction pathways to achieve tumor growth, angiogenesis and metastasis. Therefore, transmembrane proteins represent pivotal targets in cancer therapy. It would be advantageous to discover novel transmembrane proteins or polypeptides, and their corresponding polynucleotides that can be targets for production of therapies.

Transmembrane proteins that are attractive targets for therapeutic intervention include proteins that are differentially expressed on the surface of cancer or other cells, which are desirable to be eliminated or the activities of which are desirable to be inhibited, such as activated T cells in inflammatory or autoimmune diseases or conditions. This also includes transmembrane proteins that are differentially expressed on the surface of cancer cells but not on the surface of normal tissues, such as kidney and pancreas. Such therapeutic interventions include the production of antibodies that mediate antibody-dependent cellular cytotoxicity (ADCC) or complement-dependent cytotoxicity (CDC) to effect tumor cell killing, or targeting antibodies that carry a payload, such as radioisotopes or cytotoxic agents, to effect target cell killing.

Transmembrane proteins with non-transmembrane regions, i.e. nucleotides or polypeptides positioned extracellularly, cytoplasmically, or luminally to the membrane, can be useful as antibody targets for the diagnosis, prophylaxis and treatment of disorders, such as cancer. Transmembrane proteins that possess a ligand binding extracellular portion exposed on a cell surface and an intracellular portion that interacts with other cellular components upon activation, can be useful as transmembrane proteins to mediate intracellular responses, such as signal transduction. Defects in cell signal transduction pathways are responsible for a number of disorders, including hyperproliferative diseases such as the majority of cancers, immune disorders, and many inflammatory conditions, including, but not limited to, Crohn's disease (Geffen and Man, 2002; Van Den Blink et al., 2002; Lodish 1999).

Transmembrane proteins can be, e.g., receptor proteins. A receptor is a polypeptide that binds to a specific signaling molecule and initiates a cellular response. Receptors can be present on the cell surface or inside the cell. Example of receptor types include G-protein-linked receptors, ion channel-linked receptors, enzyme-linked receptors, T-cell receptors, thyroid hormone receptors, retinoid receptors, nuclear hormone receptors, and the related category of steroid hormone receptors, e.g., cortisol receptors (Alberts et al., 1994).

G-protein-linked receptors transduce extracellular signals into intracellular responses by interacting with guanine nucleotide binding proteins. The same ligand can activate many different G-protein-linked receptors. G-protein-linked receptors mediate cellular responses to a diverse range of signaling molecules, including hormones, neurotransmitters, and local mediators, which are varied in structure and function, and encompass proteins and small peptides, as well as amino acids and their derivatives, and fatty acids and their derivatives. Many signaling molecules are active at low concentrations, and their receptors often bind with high affinity. Examples of G-protein-linked receptors include, but are not limited to, rhodopsins, olfactory receptors, and β-adrenergic receptors.

Ion channel-linked receptors are involved in synaptic signaling. These receptors regulate ion channels, to which they are linked. Some respond to signals from neurotransmitters, e.g., acetylcholine, serotonin, GABA, and glycine. A common mechanism of action for ion channel-linked receptors is to transiently open or close their respective ion channel, transiently changing the permeability of the membrane in which they reside to a specific ion or ions.

Enzyme-linked receptors can be linked to enzymes or can function as enzymes. Their ligand binding site is commonly on one side of the membrane, e.g., an extracellular domain, and the catalytic site is on the other, e.g., a cytoplasmic domain. Transmembrane tyrosine-specific protein kinase receptors for growth and differentiation factors are enzyme-linked receptors; examples include receptors for epidermal growth factor (EGF), platelet-derived growth factor (PDGF), fibroblast growth factors (FGFs), hepatocyte growth factors (HGF), insulin, insulin like growth factor-1 (IGF-1), nerve growth factor (NGF), vascular endothelial growth factor (VEGF), and macrophage colony stimulating factor (M-CSF).

T-cell receptors are membrane proteins comprised of two disulfide-linked polypeptide chains, each with two immunoglobulin-like domains. They display a similarity to antibodies in that they have a variable amino-terminal region and a constant carboxyl-terminal region which is coded for by variable, joining, and constant region genes (Wei et al., 1997; Alberts et al., 1994). Rearrangement of T-cell receptor genes have been associated with human T-cell leukemias (Fisch et al., 1993).

Receptors are involved in cellular processes that regulate growth and differentiation. Their dysregulation can lead to by hyperproliferative conditions, and they are common therapeutic targets. For example, the EGF receptor is aberrantly activated in neoplasia, especially in tumors of epithelial origin. EGF receptor antagonists can successfully treat some of these tumors, either alone or in combination with chemotherapy or ionizing radiation (Kari et al., 2003). The progesterone receptor, an intracellular steroid hormone receptor, plays a role in the development and function of the mammary gland, the uterus, and the ovary. Mutation or aberrant expression of the progesterone receptor, or its regulatory molecules, can affect its normal function and lead to cancer (Gao and Nawaz, 2002).

Receptors are also involved in cellular processes that regulate inflammation and immunity. For example, they mediate immune and inflammatory responses, and function in host defense. (O'Neill, 2002). Their activation can lead to the activation of signaling cascades, e.g., pathways involving transcription factors and protein kinases, resulting in an inflammatory response (O'Neill, 2002). Another mechanism by which receptors regulate inflammation and immunity is by their selective expression, at discrete stages of differentiation, by cells involved in the inflammatory response. For example, expression of the triggering receptor expressed on myeloid cells (TREM-1) and the myeloid DAP12-associating lectin (MDL-1) are correlated with myelomonocytic differentiation. These receptors are more highly expressed in differentiated cells, are involved in monocyte activation and the inflammatory response, and are expressed at a lower level in malignant compared to normal cells (Gingras et al., 2002).

Receptors and fragments of receptors can be used as therapeutics. For example, a ligand-binding portion, an effector-binding portion, and a kinase or phosphatase domain or consensus sequence can comprise fragments that can function as agonists or antagonists to enhance or reduce, e.g., ligand binding to the natural receptors, or effector function by the natural receptors. Transmembrane proteins with extracellular fragments that can be cleaved can be useful as secreted proteins to effect ligand/receptor binding so as to mediate intracellular responses, such as signal transduction.

Transmembrane proteins can be membrane transport proteins. A membrane transport protein is an integral transmembrane protein that aids one or more molecules across a cell membrane. Most, if not all, types of molecules are transported across membranes, including proteins, ions, and fatty acids (Schaffer and Lodish, 1994). Even molecules such as water and urea, which can diffuse across pure phospholipid bilayers, are frequently accelerated by transport proteins. Transporters clear cells of toxins and confer drug resistance on tumor lines (Ramalho-Santos et al., 2002). The rate of transport varies considerably among membrane transport proteins. Membrane transport proteins function in the plasma membrane and in intracellular organellar membranes, including the nuclear, mitochondrial, lysosomal, and vesicular membranes.

Membrane transport proteins can have either a broad or a narrow range of specificity for the transported substance. In mammalian cells, nucleoside transport across membranes is mediated by broad specificity transporters. Nucleoside transport plays a role in such diverse cellular functions as nucleotide synthesis, neurotransmission, and platelet aggregation. Nucleoside transporters carry chemotherapeutic nucleosides, and are a target of interest in chemotherapeutic and cardiac drug design (Griffiths et al., 1997; Ku et al., 1990).

Carriers are another class of membrane transport proteins; they bind to a solute and transport it across the membrane by undergoing a series of conformational changes. In contrast to channel proteins, carriers bind only one, or a few, substrate molecules at a time; after binding substrate molecules, they undergo a conformational change such that the bound substrate molecules, and only those molecules, are transported across the membrane. Carriers transport a wide variety of molecules, including fatty acids across the plasma membrane (Schaffer and Lodish, 1994); purines, pyrimidines, and components of nucleosides across the nuclear membrane, and adenine nucleotides across the inner mitochondrial membrane (Battini et al., 1997).

Membrane transport proteins, such as those expressed in cancer cells, are useful as targets for therapeutic intervention, for example, in the screening for small molecule inhibitors. Inhibition of membrane transport, as indicated above, may make cancer cells more susceptible to chemotherapy.

### DISCLOSURE OF THE INVENTION

The invention provides novel nucleic acid molecules, amino acid molecules, antibodies, and antibody targets that correspond to the human gene region KIAA0779. These nucleic acid and amino acid molecules can be expressed in cell free systems or in recombinant host cells. The antibody targets include the non-transmembrane regions of herein disclosed polypeptides. These novel nucleic acid molecules, amino acid molecules, antibodies, and antibody targets find use in the diagnosis and treatment of proliferative, inflammatory, immune related, and metabolic disorders.

### BRIEF DESCRIPTION OF THE TABLES AND DRAWINGS

Table 1 identifies the human cDNA clones of the invention. Each of the sequences of the invention is identified by an internal reference number (FP ID). Table 1 correlates this reference number with each of the sequences of the invention, as shown in the Sequence Listing. Each sequence is identified by its FP ID number, and one or more SEQ ID NOS. corresponding to the nucleotide coding sequence (SEQ ID NO. (N1)), a SEQ ID NO. corresponding to the encoded polypeptide sequence (SEQ ID NO. (P1)), a SEQ ID NO. corresponding to the complete polynucleotide sequence, including both coding and non-coding regions (SEQ ID NO. (N1)), and a Source ID designation for the source of each human cDNA clone and/or fragment thereof. SEQ ID NOS. 1-2, 4-5, 7-8, 17-18, 31-32, and 35-36 correlate with the entire polynucleotides, coding regions, and polypeptides of the novel clones of the invention. SEQ ID NOS. 3, 6, 9, 19, 33, and 37 correlate with the entire polynucleotides, coding regions, and polypeptides of KIAA0779-related sequences in the public domain. SEQ ID NOS. 10-16 and 20-30 correspond to the non-transmembrane regions of their respective Source ID. Finally, SEQ ID NOS. 34 and 38 correspond to the sequences of the probes that map to clones discussed herein, and were used to determine gene expression.

Table 2 shows the structural characteristics of the novel Group 1 clones of the invention, CLN00539416.a and CLN00250082.b, and NCBI sequence 24980850:24980849. Table 2 lists the FP II) and the Source ID of each and species the predicted length of each protein (Predicted Protein Length), expressed as the predicted number of amino acid residues. Table 2 sets forth the coordinate positions of the amino acid residues comprising the coordinate positions of the amino acid residues comprising the mature protein sequences (Mature Protein Coords.). They begin at amino acid 1, indicating that these proteins do not comprise signal peptides. Table 2 shows that these proteins are transmembrane proteins and indicates the number of transmembrane sequences of each (TM). Finally, Table 2 provides the coordinates of the transmembrane and non-transmembrane sequences of the polypeptides. The transmembrane coordinates (TM Coords) refer to the transmembrane regions and are listed in terms of the amino acid residues beginning with “1” at the N-terminus of the polypeptide. The non-transmembrane coordinates (non-TM Coords) refer to the amino acids that do not comprise the transmembrane region; these can include extracellular, cytoplasmic, and luminal sequences, and are also listed in terms of the amino acid residues beginning with “1” at the N-terminus of the polypeptide.

Table 3 shows the structural characteristics of the novel Group 2 clones of the invention, CLN00219153.a and CLN00149041.a, as well as NCBI sequence 24980850:24980849, which spans the KIAA0779 gene region. Table 3 lists the FP ID and the Source ID of each and specifies the predicted length of each protein (Pred. Protein Length), expressed as the predicted number of amino acid residues. Table 3 sets forth the coordinate positions of the amino acid residues comprising the coordinate positions of the amino acid residues comprising, the matures protein sequences (Mature Protein Coords.). They also begin at amino acid 1, indicating that these proteins do not comprise signal peptides. Table 3 shows that CLN00219153.a and 24980850:24980849 are transmembrane proteins with two transmembrane sequences (TM), and that CLN00149041.a does not possess a transmembrane region. Table 3 provides the coordinates of the trans membrane (TM Coords.) and non-transmembrane (N0n-TM Coords.) sequences of the polypeptides, as discussed above. Finally, Table 3 shows that CLN00219153.a can be expressed with an alternative set of transmembrane coordinates (Alt. TM Coords.) (Alt. Non-TM Coords.).

Tables 4 and 5 present the expression profile of probe 227356_at in non-cancerous and cancerous tissues, respectively. The probe was used to interrogate a proprietary database from GeneLogic to determine the expression of these novel clones, as further described in Example 1. Probe 227356.at maps to the clones CLN00387959 and CLN00250082. The probe is overexpressed in malignant, compared to normal, bladder, breast, cervix, endometrium, squamous lunge, tumors, ovary, prostate, and skin. The greatest degree of overexpression is observed in cervix, lung, and ovary, compared to normal tissues. The probe is underexpressed in malignant, compared to normal, pancreas.

Tables 6 and 7 present the expression profile of probe 213349_at in non-cancerous and cancerous tissues, respectively. The probe was used to interrogate a proprietary database from GeneLogic to determine the expression of these novel clones, as further described in Example 1. Probe 213349_at maps to clone CLN00219153.

Tables 4-7 list the normal tissues that were examined with the probe (Tissues), and the number of independent samples of each tissue type (Samples). The percentage of the samples that express the probe is shown (% Expression). They present the approximate median level of expression on an arbitrary relative scale from 0-1200. Finally, they present data describing the range of the level of expression of these clones.

FIG. 1 compares the amino acid sequences of Group 1 by aligning them using clustal format for T-COFFEE Version_{—}1.37, CPU=0.00 sec, SCORE=100, Nseq=3, and Len=747.

FIG. 2 compares the amino acid sequences of Group 2 by aligning them using clustal format for T-COFFEE Version_{—}1.37 with the parameters CPU=0.00 sec, SCORE=79, Nseq=3, Len=653. The asterisks (*) indicate shared amino acid residues. The colons (:) indicate conservative amino acid changes. The periods (.) represent non-conservative amino acid changes.

FIG. 3 shows an exon map of the sequences of the invention. FIG. 3A shows the relative differences in the distances among the exons. FIG. 3B shows the positions of the exons in the indexed human genome.

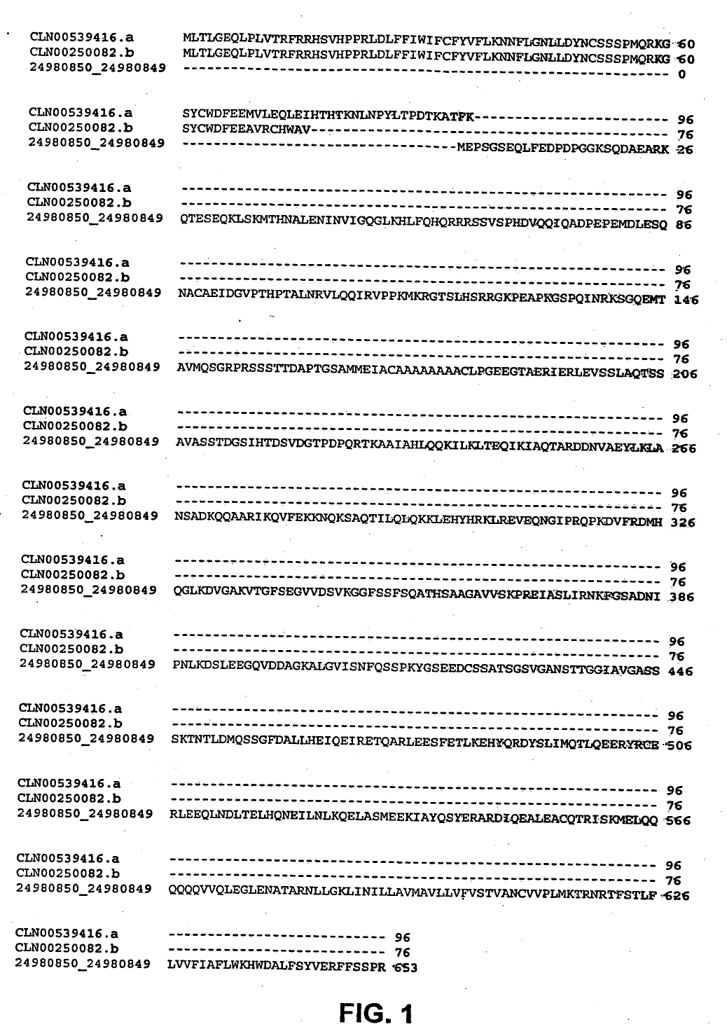

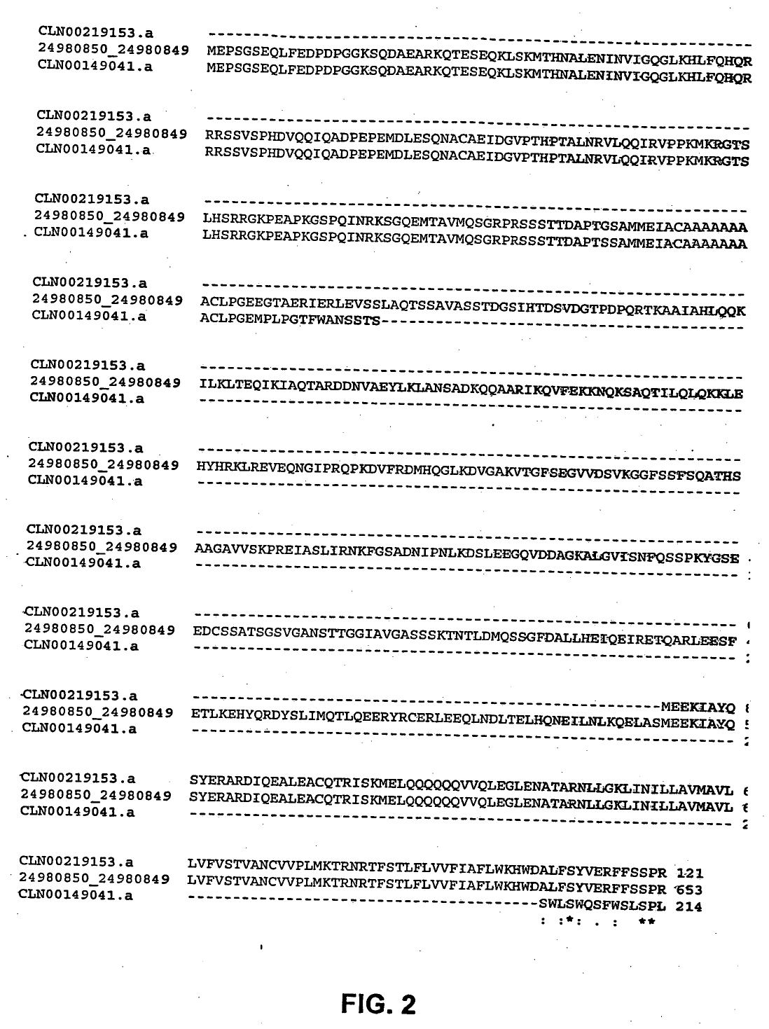

### INDUSTRIAL APPLICABILITY

The present polypeptides, polynucleotides, and modulators find use in a number of diagnostic, prophylactic, and therapeutic applications. The polynucleotides and polypeptides of the invention can be detected by methods provided herein; these methods are useful in diagnosis, and can be accomplished by the use of diagnostic kits. The polynucleotides and polypeptides of the invention are useful for production of therapeutics for treating a variety of disorders, including cancer, proliferative disorders, inflammatory disorders, immune disorders, viral disorders, and other metabolic disorders. These include therapeutic vaccines in the form of nucleic acid or polypeptide vaccines, such as cancer vaccines, where the vaccines can be administered alone, such as naked DNA, or can be facilitated, such as via viral vectors, microsomes, or liposomes. Therapeutics antibodies include those that are administered alone or in combination with cytotoxic agents, such as radioactive or chemotherapeutic agents.

In particular, the polypeptides, polynucleotides, and modulators of the present invention can be used to treat cancers, including, but not limited to, cancers of the prostate, breast, bone, soft tissue, liver, kidney, ovary, cervix, skin, pancreas, and brain, as well as leukemias, lymphomas, lung cancers such as adenocarcinomas and squamous cell carcinoma, and cancers of gastrointestinal organs such as stomach, colon, and rectum. Further, the polypeptides, polynucleotides, and modulators of the present invention can be used to treat inflammatory, immune, viral, and metabolic diseases, disorders, syndromes, or conditions.

The present polynucleotides, polypeptides, and modulators find use in therapeutic agent screening/discovery applications, such as screening for receptors or competitive ligands, for use, for example, as small molecule therapeutic drugs. Also provided are methods of modulating a biological activity of a polypeptide and methods of treating associated disease conditions, particularly by administering modulators of the present polypeptides, such as specific antibodies, small molecule modulators, and antisense molecules.

### MODES FOR CARRYING OUT THE INVENTION

Definitions

The terms “nucleic acid molecule,” “polynucleotide,” “nucleotide,” “nucleic acid,” “polynucleic molecule,” “nucleotide molecule,” “nucleic acid sequence,” “polynucleotide sequence,” and “nucleotide sequence” are used interchangeably herein to refer to polymeric forms of nucleotides of any length. The novel polynucleotides herein include those shown in the Table and Sequence Listing and biologically active fragments thereof. The polynucleotides also include modified, labeled, and degenerate variants of the nucleic acid sequences, as well as nucleic acid sequences that are substantially similar or homologous to nucleic acids encoding the subject proteins.

A “complement” of a nucleic acid molecule is a one that is comprised of its complementary base pairs. Deoxyribonucleotides with the base adenine are complementary to those with the base thymidine, and deoxyribonucleotides with the base thymidine are complementary to those with the base adenine. Deoxyribonucleotides with the base cytosine are complementary to those with the base guanine, and deoxyribonucleotides with the base guanine are complementary to those with the base cytosine. Ribonucleotides with the base adenine are complementary to those with the base uracil, and deoxyribonucleotides with the base uracil are complementary to those with the base adenine. Ribonucleotides with the base cytosine are complementary to those with the base guanine, and deoxyribonucleotides with the base guanine are complementary to those with the base cytosine.

A “promoter” is a nucleotide sequence present in DNA, to which RNA polymerase binds to begin transcription. The term includes a DNA regulatory region capable of binding RNA polymerase in a mammalian cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

“Sequence identity,” “sequence homology,” “homology,” “sequence similarity,” and “percent sequence identity,” used interchangeably herein, describe the degree of relatedness between two polynucleotide or polypeptide sequences. In general, “identity” means the exact match-up of two or more nucleotide sequences or two or more amino acid sequences, where the nucleotide or amino acids being compared are the same. Also, in general, “similarity” or “homology” means the exact match-up of two or more nucleotide sequences or two or more amino acid sequences, where the nucleotide or amino acids being compared are either the same or possess similar chemical and/or physical properties. The terms also refer to the percentage of the “aligned” bases (for the polynucleotides) or amino acid residues (for the polypeptides) that are identical when the sequences are aligned. Sequences can be aligned in a number of different ways and sequence similarity can be determined in a number of different ways. For example, the bases or amino acid residues of one sequence can be aligned to a gap in the other sequence, or they can be aligned only to another base or amino acid residue in the other sequence. A gap can range anywhere from one nucleotide, base, or amino acid residue to multiple exons in length, up to any number of nucleotides or amino acid residues. Further, sequence scan be aligned such that nucleotides (or bases) align with nucleotides, nucleotides align with amino acid residues, or amino acid residues align with amino acid residues.

A “nucleic acid hybridization reaction” is one in which single strands of DNA or RNA randomly collide with one another, and bind to each other only when their nucleotide sequences have some degree of complementarity. The solvent and temperature conditions can be varied in the reactions to modulate the extent to which the molecules can bind to one another. Hybridization reactions can be performed under different conditions of “stringency.” The “stringency” of a hybridization reaction as used herein refers to the conditions (e.g., solvent and temperature conditions) under which two nucleic acid strands will either pair or fail to pair to form a “hybrid” helix.

A vector is a plasmid that can be used to transfer DNA sequences from one organism to another or to express a gene of interest.

The term “host cell” includes an individual cell, cell line, cell culture, or in vivo cell, which can be or has been a recipient of any polynucleotides or polypeptides of the invention, for example, a recombinant vector, an isolated polynucleotide, antibody or fusion protein. Host cells include progeny of a single host cell, and the progeny may not necessarily be completely identical (in morphology, physiology, or in total DNA, RNA, or polypeptide complement) to the original parent cell due to natural, accidental, or deliberate mutation and/or change. Host cells can be prokaryotic or eukaryotic, including mammalian, insect, amphibian, reptile, crustacean, avian, fish, plant and fungal cells. A host cell includes ells transformed, transfected, transduced, or infected in vivo or in vitro with a polynucleotide of the invention, for example, a recombinant vector. A host cell which comprises a recombinant vector of the invention may be called a “recombinant host cell.”

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term includes single chain protein as well as multimers.

An “isolated,” “purified,” or “substantially isolated” polynucleotide or polypeptide, or a polynucleotide or polypeptide in “substantially pure form,” in “substantially purified form,” in “substantial purity,” or as an “isolate,” is one that is substantially free of the sequences with which it is associated in nature, or other nucleic acid or polypeptide sequences respectively that do not include a sequence or fragment of the subject polynucleotides.

A “biologically active” entity, or an entity having “biological activity,” is one having structural, regulatory, or biochemical functions of a naturally occurring molecule or any function related to or associated with a metabolic or physiological process.

The term “antibody” refers to protein generated by the immune system that is capable of recognizing and binding to a specific antigen; antibodies are commonly known in the art. An “epitope” is the site of an antigenic molecule to which an antibody binds.

An antigen is a substance that provokes an immune response.

An immunoassay is a process that identifies and/or measures the specific antigen or antibody in a sample by observing the interaction between one or more antigen and one or more antibody.

The term “modulate” encompasses an increase or a decrease, a stimulation, inhibition, or blockage in the measured activity when compared to a suitable control. “Modulation” of expression levels includes increasing the level and decreasing the level of a mRNA or polypeptide of interest encoded by a polynucleotide of the invention when compared to a control lacking the agent being tested. In some embodiments, agents of particular interest are those which inhibit a biological activity of a subject polypeptide, and/or which reduce a level of a subject polypeptide in a cell, and/or which reduce a level of a subject mRNA in a cell and/or which reduce the release of a subject polypeptide from a eukaryotic cell. In other embodiments, agents of interest are those that increase polypeptide activity.

An “agent which modulates a biological activity of a subject polypeptide,” as used herein, describes any substance, synthetic, semi-synthetic, or natural, organic or inorganic, small molecule or macromolecular, pharmaceutical or protein, with the capability of altering a biological activity of a subject polypeptide or of a fragment thereof, as described herein. Generally, a plurality of assay mixtures is run in parallel with different agent concentrations to obtain a differential response to the various concentrations. Typically, one of these concentrations serves as a negative control, i.e., at zero concentration or below the level of detection. The biological activity can be measured using any assay known in the art.

A “pharmaceutically acceptable carrier” is a non-toxic solid, semisolid or liquid filler, diluent, encapsulating material or formulation auxiliary of any conventional type. A pharmaceutically acceptable carrier is non-toxic to recipients at the dosages and concentrations employed and is compatible with other ingredients of the formulation. For example, the carrier for a formulation containing polypeptides would not normally include oxidizing agents and other compounds that are known to be deleterious to polypeptides.

Nucleic Acids, Polypeptides, and Nucleic Acid and Polypeptide Compositions

As discussed above, and illustrated in the Tables and Figures, the invention provides nucleic acids and polypeptides that correspond to full-length cDNA clones, their translation and transcription products, and fragments thereof. These nucleic acids and polypeptides have sequences that correspond to the heretofor uncharacterized human gene region designated KIAA0779. The human sequences in the public domain with the greatest similarity to the sequences of the invention were determined by comparison of the sequences of the invention with the public National Center for Information Biotechnology (NCBI) database.

The novel DNA clones of the invention were isolated from libraries made from various types of tissues. For example, CLN00219153.a was isolated from kidney tissue and CLN00250082.b was isolated from tonsil tissue. The invention also provides novel antibody targets comprising the KIAA proteins, e.g., the KIAA protein 24989850, including non-transmembrane regions thereof, as identified by the methods and probes disclosed herein.

The protein with the greatest sequence homology to CLN00219153.a is a polypeptide with the NCBI accession number gi|39930343|ref|NP_{—}055823.1| and the annotation KIAA0779 protein [Homo sapiens] gi|34364785|emb|CAE45831.1| hypothetical protein [Homo sapiens]. The polypeptide gi|39930343|ref|NP_{—}055823.1| is 329 amino acids in length. CLN00219153.a, a 121 amino acid protein, has 121 amino acids that are identical with gi|39930343|ref|NP_{—}055823.1|, thus, they are 100% identical over the portion of gi|39930343|ref|NP_{—}055823.1| which was queried with CLN00219153.a. This represents 37% identity over the full length of gi|39930343|ref|NP_{—}055823.1|.

The protein with the greatest sequence homology to CLN00149041.a is a polypeptide with the NCBI accession number gi|24980850|gb|AAH39859.1| and the annotation KIAA0779 protein [Homo sapiens]. The polypeptide gi|24980850|gb|AAH39859.1| is 653 amino acids in length. CLN0014904.a, a 214 amino acid protein, has 190 amino acids that are identical with gi|24980850|gb|AAH39859.1|, thus, they are 89% identical over the portion of gi|24980850|gb|AAH39859.1| which was queried with CLN00149041.a. This represents 29% identity over the full length of gi|24980850|gb|AAH39859.1|.

The protein with the greatest sequence homology to the NCBI sequence 24980850:24980849 is a polypeptide with the NCBI accession number gi|24980850|gb|AAH39859.1| and the annotation KIAA0779 protein [Homo sapiens]. The polypeptide gi|24980850|gb|AAH39859.1| is 653 amino acids in length. 24980850:24980849, a 653 amino acid protein, has 653 amino acids that are identical with gi|24980850|gb|AAH39859.1|, thus, they are 100% identical over both the portion of gi|24980850|gb|AAH39859.1| which was queried with 24980850:24980849 and over the full length of gi|24980850|gb|AAH39859.1|.

Nucleic Acids

The nucleic acid molecules of the invention can comprise polynucleotides that contain deoxyribonucleotides, ribonucleotides, and/or their analogs or derivatives. For example, nucleic acids can be naturally occurring DNA or RNA, or can be synthetic analogs, as known in the art. The terms also encompass genomic DNA, genes, gene fragments, exons, introns, regulatory sequences or regulatory elements (such as promoters, enhancers, initiation and termination regions, other control regions, expression regulatory factors, and expression controls), DNA comprising one or more single-nucleotide polymorphisms (SNPs), allelic variants, isolated DNA of any sequence, and cDNA. The terms also encompass mRNA, tRNA, rRNA, ribozymes, splice variants, antisense RNA, antisense conjugates, RNAi, and isolated RNA of any sequence.

Nucleic acid molecules of the invention include oligonucleotides, which are generally polynucleotides of between about 5 and about 100 nucleotides of single- or double-stranded nucleic acids. For the purposes of this disclosure, there is no upper limit to the length of an oligonucleotide. Oligonucleotides are also known as oligomers or oligos and can be isolated from genes, or chemically synthesized by methods known in the art.

Nucleic acid molecules of the invention also encompass recombinant polynucleotides, heterologous polynucleotides, branched polynucleotides, labeled polynucleotides, hybrid DNA/RNA, polynucleotide constructs, vectors comprising the subject nucleic acids, nucleic acid probes, primers, and primer pairs. The polynucleotides can comprise modified nucleic acid molecules, with alterations in the backbone, sugars, or heterocyclic bases, such as methylated nucleic acid molecules, peptide nucleic acids, and nucleic acid molecule analogs, which may be suitable as, for example, probes if they demonstrate superior stability and/or binding affinity under assay conditions. Analogs of purines and pyrimidines, including radiolabeled and fluorescent analogs, are known in the art. The polynucleotides can have any three-dimensional structure, and can perform any function, known or as yet unknown. The terms also encompass single-stranded, double-stranded and triple helical molecules that are either DNA, RNA, or hybrid DNA/RNA and that may encode a full-length gene or a biologically active fragment thereof.

Biologically active fragments of polynucleotides can encode the polypeptides herein, as well as anti-sense and RNAi molecules. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, or when it has therapeutic value in alleviating a disease condition, or when it has prophylactic value in inducing an immune response to the molecule, or when it has diagnostic value in determining the presence of the molecule, such as a biologically active fragment of a polynucleotide that can be detected as unique for the polynucleotide molecule, or that can be used as a primer in PCR. Thus, the full length polynucleotides herein may be treated with enzymes, such as Dicer, to generate a library of short RNAi fragments which are within the scope of the present invention.

The nucleic acid molecules herein include splice variants, i.e., an types of RNAs transcribed from a given gene that when processed collectively encode plural protein isoforms. For example, nucleic acid molecules of the invention can be produced by alternative splicing, i.e., all types of RNA processing that lead to expression of plural protein isoforms from a single gene. Some genes are first transcribed as long mRNA precursors that are then shortened by a series of processing steps to produce the mature mRNA molecule. One of these steps is RNA splicing, in which the intron sequences are removed from the mRNA precursor. A cell can splice the primary transcript in different ways, making different splice variants, and thereby making different polypeptide chains from the same gene, or from the same mRNA molecule. Splice variants can include, for example, exon insertions, exon extensions, exon truncations, exon deletions, alternatives in the 5′ untranslated region and alternatives in the 3′ untranslated region.

The novel polynucleotides herein include those shown in the Table and Sequence Listing and biologically active fragments thereof. The polynucleotides also include modified, labeled, and degenerate variants of the nucleic acid sequences, as well as nucleic acid sequences that are substantially similar or homologous to nucleic acids encoding the subject proteins.

The present invention provides novel cDNA molecules, novel genes encoding proteins, the encoded proteins, and fragments complements, and homologs thereof. Specifically, it provides an isolated nucleic acid molecule comprising at least one polynucleotide sequence with SEQ ID NOS.: 1-2, 4-5, 31-32, and 35-36; sequences that hybridize to these sequences of under high stringency conditions; sequences having at least 80% sequence identity to the sequences of SEQ ID NOS.: 1-2, 4-5, 31-32, and 35-36 or sequences that hybridize to them under high stringency conditions; complements of any of these sequences, or biologically active fragments of any of the above-listed sequences. These nucleic acid molecules can be DNA or RNA molecules. These nucleic acid molecules can be a double-stranded isolated nucleic acid molecule comprising an above-described nucleic acid molecule and its complement. These nucleic acid molecules can also comprise a sequence that encodes a polypeptide or a biologically active fragment of the polypeptide.

Non-limiting embodiments of nucleic acid molecules include genes or gene fragments, exons, introns, mRNA, tRNA, rRNA, siRNA, ribozymes, antisense cDNA, recombinant polynucleotides, branched polynucleotides, plasmids, vectors, isolated DNA of any sequence, isolated RNA of any sequence, nucleic acid probes, and primers. Nucleic acid molecules include splice variants of an mRNA. Nucleic acids can be naturally occurring, e.g. DNA or RNA, or can be synthetic analogs, as known in the art. Such analogs are suitable as probes because they demonstrate stability under assay conditions. A nucleic acid molecule can also comprise modified nucleic acid molecules, such as methylated nucleic acid molecules and nucleic acid molecule analogs. Analogs of purines and pyrimidines are known in the art.

Nucleic acid compositions can comprise a sequence of DNA or RNA, including one having an open reading frame that encodes a polypeptide and is capable, under appropriate conditions, of being expressed as a polypeptide. The nucleic acid compositions also can comprise fragments of DNA or RNA. The term encompasses genomic DNA, cDNA, mRNA, splice variants, antisense RNA, RNAi, siRNA, DNA comprising one or more single-nucleotide polymorphisms (SNP), and vectors comprising nucleic acid sequences of interest. Nucleic acid compositions also include, for example, vectors, including plasmids, cosmids, viral vectors (e.g., retrovirus vectors such as lentivirus, adenovirus and the like), human, yeast, bacterial, P1-derived artificial chromosomes (HAC's, YAC's, BAC's, PAC's, etc), and mini-chromosomes, in vitro host cells, in vivo host cells, tissues, organs, allogenic or congenic grafts or transplants, multicellular organisms, and chimeric, genetically modified, or transgenic animals comprising a subject nucleic acid sequence.

The isolated nucleic acid molecules of the invention an substantially free of the sequences with which it is associated in nature, or other nucleic acid sequences that do not include a sequence or fragment of the subject polynucleotides. By substantially free is meant that less than about 90%, less than about 80%, less than about 70%, less than about 60%, or less than about 50% of the composition is made up of materials other than the isolated polynucleotide. For example, the isolated polynucleotide is at least about 50%, at least about 60%, at least about 70%, at least about 80%, at least about 90%, at least about 95%, at least about 97%, or at least about 99% free of the materials with which it is associated in nature. For example, an isolated polynucleotide may be present in a composition wherein at least about 50%, at least about 60%, at least about 70%, at lest about 80%, at least about 90%, at least about 95%, at least about 97%, at least about 99% of the total macromolecules (for example, polypeptides, fragments thereof, polynucleotides, fragments thereof, lipids, polysaccharides, and oligosaccharides) in the composition is the isolated polynucleotide. Where at least about 99% of the total macromolecules is the isolated polynucleotide, the polynucleotide is at least about 99% pure, and the composition comprises less than about 1% contaminant.

As used herein, an isolated, purified or substantially isolated polynucleotide, or a polynucleotide in substantially pure form, in substantially purified form, in substantial purity, or as an isolate, also refers to recombinant polynucleotides, modified, degenerate and homologous polynucleotides, and chemically synthesized polynucleotides, which, by virtue of origin or manipulation, are not associated with all or a portion of a polynucleotide with which it is associated in nature, are linked to a polynucleotide other than that to which it is linked in nature, or do not occur in nature. For example, the subject polynucleotides are generally provided as other than on an intact chromosome, and recombinant embodiments are typically flanked by one or more nucleotides not normally associated with the subject polynucleotide on a naturally-occurring chromosome.

The novel cDNA clones of the invention were derived from total RNA isolated from normal or diseased tissues and from normal or treated cells, e.g., kidney, as described above. These RNA samples were transcribed into cDNA using technology described by RIKEN and others, including, methods of capturing the 5′ ends of DNA (“CAP trapping”) and methods to eliminate secondary structure in the mRNA using trehalose so that the entire molecule can be reverse transcribed (WO 02/28876; WO 02/070720; U.S. Pat. No. 6,627,399; U.S. Pat. No. 6,458,756; U.S. Pat. No. 6,372,437; U.S. Pat. No. 6,365,350; U.S. Pat. No. 3,344,345; U.S. Pat. No. 6,342,387, U.S. Pat. No. 6,333,156; U.S. Pat. No. 6,294,337; U.S. Pat. No. 6,265,569; U.S. Pat. No. 6,221,599; U.S. Pat. No. 6,174,669; U.S. Pat. No. 6,143,528; U.S. Pat. No. 6,074,824; and U.S. Pat. No. 6,013,488).

Libraries of the transcribed cDNA were compiled, and samples of approximately three 384-well plates from each library were sequenced at their 5′ end. Using the diversity of the library as represented by the sample as the criteria, the 5′ ends of as many as 10,000 clones from each library were sequenced. This 5′ end sequence information was the basis of an analysis that provided a clustered organization of the clones. The clusters were based on a map of the human genome including all known human genes and all known human expressed sequence tags. Multiple sequences mapping to the same locus were identified as belonging to one cluster. A cluster may include splice variants. Clones mapping to a locus comprising no previously identified genes are identified herein. These cDNA clones represent novel genes belonging to novel gene clusters. Further, samples of some of the members of the transcribed cDNA libraries were compiled, and sequenced at their 3′ end, as well as their 5′ end. A subset of these possessed contiguous 5′ end sequence and 3′ end sequence. These were assembled into full length sequences, and are identified herein as the novel cDNA clones of the invention, and described herein.

In some embodiments, a polynucleotide of the invention comprises a nucleotide sequence of at least about 10, at least about 15, at least about 18, at least about 20, at least about 25, at least about 30, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1100, at least about 1200, at least about 1300, at east about 1400, at least about 1500, at least about 2000, or at least about 3000 contiguous nucleotides of any one of the sequences shown in SEQ ID NOS.: 1-6 and 31-38, or the coding region thereof, or a complement thereof.

The nucleic acids of the subject invention can encode all or a part of the subject proteins. Double or single stranded fragments can be obtained from the DNA sequence by chemically synthesizing oligonucleotides in accordance with conventional methods, for example by restriction enzyme digestion or polymerase chain reaction (PCR) amplification. The use of the polymerase chain reaction has been described (Saiki et al., 1985) and current techniques have been reviewed (Sambrook et al., 1989; McPherson et al. 2000; Dieffenbach and Dveksler, 1995). For the most part, DNA fragments will be of at least about 5 nucleotides, at least about 8 nucleotides, at least about 10 nucleotides, at least about 15 nucleotides, at least about 18 nucleotides, at least about 20 nucleotides, at least about 25 nucleotides, at least about 30 nucleotides, or at least about 50 nucleotides, at least about 75 nucleotides, or at least about 100 nucleotides. Nucleic acid compositions that encode at least six contiguous amino acids (i.e., fragments of 18 nucleotides or more), for example, nucleic acid compositions encoding at least 8 contiguous amino acids (i.e., fragments of 24 nucleotides or more), are useful in directing the expression or the synthesis of peptides that can be used as immunogens (Lerner, 1982; Shinnick et al., 1983; Sutcliffe et al., 1983).

The nucleic acids of the invention include degenerate variants that can be translated, according to the standard genetic code, to provide an amino acid sequence identical to that translated from the nucleic acid sequences herein. For example, synonymous codons include GGG, GGA, GGC, and GGU, each encoding glycine. The nucleic acids of the invention also include those that encode variants of the polypeptide sequences encoded by the polynucleotides of the Sequence Listing. In some embodiments, these polynucleotides encode variant polypeptides that include insertions, additions, deletions, or substitutions, e.g., conservative amino acid substitutions, compared with the polypeptides encoded by the nucleotide sequences shown in SEQ ID NOS.:1-2, 4-5, 31-32, and 35-36. Conservative amino acid substitutions include serine/threonine, valine/leucine/isoleucine, asparagine/histidine/glutamine, glutamic acid/aspartic acid, etc. (Gonnet et al., 1992).

The nucleic acids of the invention further include allelic variants. They include single nucleotide polymorphisms (SNPs), which occur frequently in eukaryotic genomes (Lander, et al., 2001). The nucleotide sequence determined from one individual of a species can differ from other allelic forms present within the population. Nucleic acids of the invention include those found in disease and/or pathological variants, as described in greater detail herein.

The nucleic acids of the invention include homologs of the polynucleotides. The source of homologous genes can be any species, e.g., primate species, particularly human; rodents, such as rats, hamsters, guinea pigs, and mice; lapines; canines; felines; cattles, such as bovines, goats, pigs, sheep, and equines; crustaceans; avians, such as chickens; reptiles; amphibians; fish; insects; plants; fungi; yeast; nematodes, etc. Among mammalian species, e.g., human and mouse, homologs can have substantial sequence similarity, e.g., at least about 60% sequence identity, at least about 75% sequence identity, or at least about 80% sequence identity among nucleotide sequences. In many embodiments of interest, homology will be at least about 75%, at least about 80%, at least about 85%, at least about 90%, at least about 93%, at least about 95%, at least about 97%, or at least about 98%; in certain embodiments of interest the homology will be as high as about 99%.

Nucleic acid molecules of the invention can comprise heterologous nucleic acid sequences, i.e., nucleic acid sequences of any length other than those specified in the Sequence Listing. For example, the subject nucleic acid molecules can be flanked on the 5′ and/or 3′ ends by heterologous nucleic acid molecules of from about 1 nucleotide to about 10 nucleotides, from about 10 nucleotides to about 20 nucleotides, from about 20 nucleotides to about 50 nucleotides, from about 50 nucleotides to about 100 nucleotides, from about 100 nucleotides to about 250 nucleotides, from about 250 nucleotides to about 500 nucleotides, or from about 500 nucleotides to about 1000 nucleotides, or more in length.

Heterologous sequences of the invention can comprise nucleotides present between the initiation codon and the stop codon, including some or all of the introns that are normally present in a native chromosome. They can further include the 3′ and 5′ untranslated regions found in the mature mRNA. They can further include specific transcriptional and translational regulatory sequences, such as promoters, enhancers, etc., including about 1 kb, about 2 kb, and possibly more, of flanking genomic DNA at either the 5′ or 3′ end of the transcribed region. Genomic DNA can be isolated as a fragment of 100 kbp or smaller; and substantially free of flanking chromosomal sequence. This genomic DNA flanking the coding region, either 3′ or 5′; or internal regulatory sequences as sometimes found in introns, may contain sequences required for proper tissue and stage-specific expression.

The sequence of the 5′ flanking region can be utilized as promoter elements, including enhancer binding sites that provide for tissue-specific expression and developmental regulation in tissues where the subject genes are expressed, providing promoters that mimic the native pattern of expression. Naturally occurring polymorphisms in the promoter region are useful for determining natural variations in expression, particularly those that may be associated with disease. Promoters or enhancers that regulate the transcription of the polynucleotides of the present invention are obtainable by use of PCR techniques using human tissues, and one or more of the present primers.

Regulatory sequences can be used to identify cis acting sequences required for transcriptional or translational regulation of expression, especially in different tissues or stages of development, and to identify is acting sequences and trans-acting factors that regulate or mediate expression. Such transcription or translational control regions can be operably linked to a gene in order to promote expression of wild type genes or of proteins of interest in cultured cells, embryonic, fetal or adult tissues, and for gene therapy (Hooper, 1993).

The invention provides variants resulting from random or site-directed mutagenesis. Techniques for in vitro mutagenesis of cloned genes are known. Examples of protocols for site specific mutagenesis may be found in Gustin et al., 1993; Barany 1985; Colicelli et al., 1985; Prentki et al., 1984. Methods for site specific mutagenesis can be found in Sambrook et al., 1989 (pp. 15.3-15.108); Weiner et al., 1993; Sayers et al. 1992; Jones and Winistorfer; Barton et al., 1990; Marotti and Tomich 1989; and Zhu, 1989. Such mutated genes can be used to study structure-function relationships of the subject proteins, or to alter properties of the protein that affect its function or regulation. Other modifications of interest include epitope tagging, e.g., with hemagglutinin (HA), FLAG, or c-myc. For studies of subcellular localization, fluorescent fusion proteins can be used.

The invention also provides variants resulting from chemical or other modifications. Modifications in the native structure of nucleic acids, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters, and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids have modifications that replace the entire ribose phosphodiester backbone with a peptide linkage.

Sugar modifications are also used to enhance stability and affinity. The α-anomer of deoxyribose can be used where the base is inverted with respect to the natural β-anomer. The 2′-OH of the ribose sugar can be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine, and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Mutations can be introduced into the promoter region to determine the effect of altering expression in experimentally defined systems. Methods for the identification of specific DNA motifs involved in the binding of transcriptional factors are known in the art, for example sequence similarity to known binding motifs, and gel retardation studies (Blackwell et al., 1995; Mortlock et al., 1996; Joulin and Richard-Foy, 11995).

In an embodiment, the isolated nucleic acid molecules are from about 20 to about 30, from about 30 to about 40, from about 40 to about 50, from about 50 to about 100, or from about 100 to about 200 nucleotides in length. Generally, these nucleic acids are used in pairs in a polymerase chain reaction, where they are referred to as “forward” and “reverse” primers. Thus, in an embodiment, the invention provides a pair of isolated nucleic acid molecules, each from about 10 to about 200 nucleotides in length, the first nucleic acid molecule of the pair comprising a sequence of at least 10 contiguous nucleotides having 100% sequence identity to a nucleic acid sequence as shown in SEQ ID NOS.: 1-6, 31-38, and the second nucleic acid molecule of the pair comprising a sequence of at least 10 contiguous nucleotides having 100% sequence identity to the reverse complement of the nucleic acid sequence shown in SEQ ID NOS.:1-2, 4-5, 31-32, and 35-36, wherein the sequence of the second nucleic acid molecule is located 3′ of the nucleic acid sequence of the first nucleic acid molecule shown in SEQ ID NOS.:1-2, 4-5, 31-32, and 35-36. The primer nucleic acids are prepared using any known method, e.g., automated synthesis, and can be chosen to specifically amplify a cDNA copy of an mRNA encoding a subject polypeptide.

In some embodiments, the invention provides isolated nucleic acids that, when used as primers in a polymerase chain reaction, amplify a subject polynucleotide, or a polynucleotide containing a subject polynucleotide. The amplified polynucleotide is from about 20 to about 50, from about 50 to about 75, from about 75 to about 100, from about 100 to about 125, from about 125 to about 150, from about 150 to about 175, from about 175 to about 200, from about 200 to about 250, from about 250 to about 300, from about 300 to about 350, from about 350 to about 400, from about 400 to about 500, from about 500 to about 600, from about 600 to about 700, from about 700 to about 800, from about 800 to about 900, from about 900 to about 1000, from about 1000 to about 2000, or from about 2000 to about 3000 nucleotides or more in length.

The subject nucleic acid compositions find use in a variety of different investigative applications. Applications of interest include identifying genomic DNA sequence using molecules of the invention, identifying homologs of molecules of the invention, creating a source of novel promoter elements, identifying expression regulatory factors, creating a source of probes and primers for hybridization applications, identifying expression patterns in biological specimens; preparing cell or animal models to investigate the function of the molecules of the invention, and preparing in vitro models to investigate the function of the molecules of the invention.

The isolated nucleic acids of the invention can be used as probes to detect and characterize gross alteration in a genomic locus, such as deletions, insertions, translocations, and duplications, e.g., by applying fluorescence in situ hybridization (FISH) techniques to examine chromosome spreads (Andreeff et al., 1999). These nucleic acids are also useful for detecting smaller genomic alterations, such as deletions, insertions, additions, translocations, and substitutions (e.g., SNPs).

When used as probes to detect nucleic acid molecules capable of hybridizing with nucleic acids described in the Sequence Listing, the nucleic acid molecules can be flanked by heterologous sequences of any length. When used as probes, a subject nucleic acid can include nucleotide analogs that incorporate labels that are directly detectable, such as radiolabels or fluorescent labels, or nucleotide analogs that incorporate labels that can be visualized in a subsequent reaction.

Fluorescent labels also include a green fluorescent protein (GFP), e.g., a humanized version of a FP, e.g., wherein codons of the naturally-occurring nucleotide sequence are changed to more closely match the human codon bias; a GFP derived from Aequoria victoria or a derivative thereof, e.g., a humanized derivative such as Enhanced GFP, available commercially, e.g., from Clontech, Inc.; other fluorescent mutants of a GFP from Aequoria victoria, e.g., as described in U.S. Pat. Nos. 6,066,476; 6,020,192; 5,985,577; 5,976,796; 5,968,750; 5,968,738; 5,958,713; 5,919,445; 5,874,304; a GFP from another species such as Renilla reniformis, Renilla mulleri, or Ptilosarcus guernyi, as previously described (WO 99/49019; Peelle et al., 2001), humanized recombinant GFP (hrGFP) (Stratagene®); and any of a variety of fluorescent and colored proteins from Anthozoan species, (e.g., Matz et al., 1999).

Probes can also contain fluorescent analogs, including commercially available fluorescent nucleotide analogs that can readily be incorporated into a subject nucleic acid. These include deoxyribonucleotides and/or ribonucleotide analogs labeled with Cy3, Cy5, Texas Red, Alexa Fluor dyes, rhodamine, cascade blue, or BODIPY, and the like.

Suitable radioactive labels include, e.g., ³²P, ³⁵S, or ³H. For example, probes can contain radiolabeled analogs, including those commonly labeled with ³²P or ³⁵S, such as α-³²P-dATP, dTTP, dCTP, and dGTP; γ-³⁵S-GTP and α-³⁵S-dATP, and the like.

In some embodiments, the first and/or the second nucleic acid molecules comprise a detectable label. The label can be a radioactive molecule, fluorescent molecule or another molecule, e.g., hapten, as described in detail above. Further, the label can be a two stage system, where the amplified DNA is conjugated to another molecule, i.e., biotin, digoxin, or a hapten, that has a high affinity binding partner, i.e., avidin, antidigoxin, or a specific antibody, respectively, and the binding partner conjugated to a detectable label. The label can be conjugated to one or both of the primers. Alternatively, the pool of nucleotides used in the amplification is labeled, so as to incorporate the label into the amplification product.

The invention also includes nucleic acids that bind to any of the polynucleotides selected from SEQ ID NOS.: 1-2, 4-5, 31-32, and 35-36 under various levels of stringency. Conditions that increase stringency of both DNA/DNA and DNA/RNA hybridization reactions are widely known and published in the art. See, for example, Sambrook, 2001, and examples provided above. Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where 1×SSC is 0.15 M NaCl and 15 mM citrate buffer); and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water.

For example, high stringency conditions include hybridization in 50% formamide, 5×SSC, 0.2 μg/μl poly(dA), 0.2 μg/μl human cot1 DNA, and 0.5% SDS, in a humid oven at 42° C. overnight, followed by successive washes in 1×SSC, 0.2% SDS at 55° C. for 5 minutes, followed by washing at 0.1×SSC, 0.2% SDS at 55° C. for 20 minutes. Further examples of high stringency conditions include hybridization at 50° C. and 0.1×SSC; overnight incubation at 42° C. in a solution containing 50% formamide, 1×SSC, 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. High stringency conditions can also include aqueous hybridization (e.g., free of formamide) in 6×SSC, 1% (SDS) at 65° C. for about 8 hours (or more), followed by one or more washes in 0.2×SSC, 0.1% SDS at 65° C. Highly stringent hybridization conditions are hybridization conditions that are at least as stringent as any one of the above representative conditions. Other stringent hybridization conditions are known in the art and can also be employed to identify nucleic acids of this particular embodiment of the invention.

Conditions of reduced stringency, suitable for hybridization to molecules encoding structurally and functionally related proteins, or otherwise serving related or associated functions, are the same as those for high stringency conditions but with a reduction in temperature for hybridization and washing to lower temperatures (e.g., room temperature or from about 22° C. to 25° C.). For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature. Low stringency conditions include, for example, aqueous hybridization at 50° C. and 6×SSC and washing at 25° C. in 1×SSC.

The specificity of a hybridization reaction allows any single-stranded sequence of nucleotides to be labeled with a radioisotope or chemical and used as a probe to find a complementary strand, even in a cell or cell extract that contains millions of different DNA and RNA sequences. Probes of this type are widely used to detect the nucleic acids corresponding to specific genes, both to facilitate the purification and characterization of the genes after cell lysis and to localize them in cells, tissues, and organisms.

Moreover, by carrying out hybridization reactions under conditions of reduced stringency, a probe prepared from one gene can be used to find homologous evolutionary relatives—both in the same organism, where the relatives form part of a gene family, and in other organisms, where the evolutionary history of the nucleotide sequence can be traced. A person skilled in the art would recognize how to modify the conditions to achieve the requisite degree of stringency for a particular hybridization.

Polypeptides

In some embodiments, a polynucleotide of the invention comprises a nucleotide sequence that encodes a polypeptide comprising an amino acid sequence of at least about 5, at least about 8, at least about 10, at least about 15, at least about 18, at least about 20, at least about 25, at least about 30, at least about 50, at least about 75, at least about 100, at least about 150, at least about 200, at least about 250, at least about 300, at least about 350, at least about 400, at least about 450, at least about 500, at least about 600, at least about 700, at least about 800, at least about 900, or at least about 1000 contiguous amino acids of at least one of the sequences shown in SEQ ID NOS.:1-2, 4-5, 31-32, and 35-36 (e.g., a polypeptide encoded by at least one of the nucleotide sequences shown in SEQ ID NOS.:1-2, 4-5, 31-32, and 35-36), up to and including an entire amino acid sequence as shown in SEQ ID NOS.: 7-13, 17-27 (or as encoded by at least one of the nucleotide sequences shown in SEQ ID NOS.:1-2, 4-5, 31-32, and 35-36).

The invention provides novel polypeptides and related polypeptide compositions. Generally, a polypeptide of the invention refers to a polypeptide which has the amino acid sequence set forth in one or more of SEQ ID NOS.:7-8, 10-13, 17-18, and 20-27, as well as polypeptides comprising the amino acid sequences of SEQ ID NOS.:7-8, 10-13, 17-18, and 20-27 and polypeptides comprising an amino acid sequences which have at least 70%, at least 80%, at least 85%, at least 90%, at least 93%, at least 95%, at least 98%, or at least 99% identity to that of SEQ ID NOS.:7-8, 10-13, 17-18, and 20-27, over their entire length. A polypeptide of the invention has an amino acid sequence substantially identical to the sequence of any polypeptide encoded by a polynucleotide sequence shown in SEQ ID NOS.:1-2, 4-5, 31-32, and 35-36. The novel polypeptides of the invention include fragments thereof, and variants, as discussed in more detail below.

In an embodiment, the invention provides an isolated polypeptide comprising an amino acid sequence, wherein the amino acid sequence is chosen from SEQ ID NOS.:7-8, 10-13, 17-18, and 20-27, or a biologically active fragment thereof, or is encoded by a polynucleotide sequence chosen from SEQ ID NOS.:1-2, 4-5, 31-32, and 35-36, or a biologically active fragment thereof. These isolated polypeptides can comprise non-transmembrane regions, as encoded by a polynucleotide sequence comprising SEQ ID NOS.:1-2, 4-5, 31-32, and 35-36, or comprising amino acid sequence SEQ ID NOS.:7-8, 10-13, 17-18, and 20-27. In an embodiment of the invention, the transmembrane regions comprise extracellular regions.

In some embodiments, a polypeptide of the invention comprises at least about 5, at least about 8, at least about 10, at least about 15, at least about 18, at least about 20, at least about 25, at least about 30, at least about 50, at least about 75, at least about 100, at least about 125, at least about 200, at least about 250, at least about 300, at least about 350, at least about 400, at least about 450, at least about 500, at least about 600, at least about 700, at least about 800, at least about 900, or at least about 1000 contiguous amino acids of one or more of the sequences according to SEQ ID NOS.:7-8, 10-13, 17-18, and 20-27, up to and including the entire amino acid sequence.

Fragments of the subject polypeptides, as well as polypeptides comprising such fragments, are also provided. Fragments of polypeptides of interest will typically be at least about 5, at least about 8, at least about 10, at least about 15, at least about 18, at least about 20, at least about 25, at least about 30, at least about 50, at least about 75, at least about 100, at least about 150, at least about 200, at least about 250, or at least 300 amino acids in length or longer, where the fragment will have a stretch of amino acids that is identical to the subject protein of at least about 5, at least about 8, at least about 10, at least about 15, at least about 18, at least about 20, at least about 25, at least about 30, or at least about 50 amino acids in length.

The proteins of the subject invention (e.g., polypeptides encoded by the nucleotide sequences shown in SEQ ID NOS.:1-2, 4-5, 31-32, and 35-36, and polypeptide sequences shown in SEQ ID NOS.:7-8, 10-13, 17-18, and 20-27) have been separated from their naturally occurring environment and are present in a non-naturally occurring environment. In certain embodiments, the proteins are present in a composition where they are more concentrated than in their naturally occurring environment.

The invention provides isolated polypeptides which are substantially free of the materials with which it is associated in nature or other polypeptide sequences that do not include a sequence or fragment of the subject polypeptides. By substantially free is meant that less than about 90%, less than about 80%, less than about 70%, less than about 60%, or less than about 50% of the composition is made up of materials other than the isolated polypeptide. For example, the isolated polypeptide is at least about 50%, at least about 60%, at least about 70%, at least about 80%, at least about 90%, at least about 95%, at least about 97%, or at least about 99% free of the materials with which it is associated in nature. For example, an isolated polypeptide may be present in a composition wherein at least about 50%, at least about 60%, at least about 70%, at least about 80%, at least about 90%, at least about 95%, at least about 97%, or at least about 99% of the total macromolecules (for example, polypeptides, fragments thereof, polynucleotides, fragments thereof, lipids, polysaccharides, and oligosaccharides) in the composition is the isolated polypeptide. Where at least about 99% of the total macromolecules is the isolated polypeptide, the polypeptide is at least about 99% pure, and the composition comprises less than about 1% contaminant. As used herein, an “isolated,” “purified,” or “substantially isolated” polypeptide, or a polypeptide in “substantially pure form,” in “substantially purified form,” in “substantial purity,” or as an “isolate,” also refers to recombinant polypeptides, modified, tagged and fusion polypeptides, and chemically synthesized polypeptides, which by virtue or origin or manipulation, are not associated with all or a portion of the materials with which they are associated in nature, are linked to molecules other than that to which they are linked in nature, or do not occur in nature.

Polypeptides of the invention include conjugated proteins, fusion proteins, including, but not limited to, GST fusion proteins, fusion proteins with a heterologous amino acid sequence, fusion proteins with heterologous and homologous leader sequences, fusion proteins with or without N-terminal methionine residues, pegylated proteins, and immunologically tagged proteins. Also included in this term are variations of naturally occurring proteins, where such variations are homologous or substantially similar to the naturally occurring protein, as well as corresponding homologs from different species. Variants of polypeptide sequences include insertions, additions, deletions, or substitutions compared with the subject polypeptides. The term also includes peptide aptamers.

Variants and derivatives of native proteins that retain a desired biological activity are also within the scope of the present invention. These variants and derivatives include polypeptides substantially homologous to native proteins, but with an amino acid sequence different from that of the native protein because of one or more of a plurality of deletions, insertions, or substitutions. In an embodiment, the biological activity of a variant is essentially equivalent to the biological activity of the native protein. Variants may be obtained by mutations of native nucleotide sequences. Polypeptide-encoding DNA sequences of the present invention encompass sequences that comprise one or more additions, deletion, or substitutions of nucleotides when compared to a native DNA sequence, but that encode a protein essentially biologically equivalent to a native protein. The variant amino acid or DNA sequence preferably is at least about 70%, at least about 75%, at least about 80%, at least about 85%, at least about 90%, at least about 93%, at least about 95%, at least about 97%, at least about 98%, or at least about 99% identical to a native sequence. The degree of homology (percent identity) between a native and a mutant sequence may be determined, for example, by comparing the two sequences using computer programs commonly employed for this purpose. Homologues can comprise polypeptides of other species, including mammals, such as: primates, rodents, e.g., mice, rats, hamsters, guinea pigs; domestic animals, e.g., sheep, pig, horse, cow, goat, rabbit, dog, cat; and humans, as well as non-mammalian species, e.g., avian, reptile and amphibian, insect, crustacean, fish, plant, fungus, and protozoa. Homology can be measured, e.g., with the “GAP” program (part of the Wisconsin Sequence Analysis Package available through the Genetics Computer Group, Inc. (Madison Wis.)), where the parameters are: Gap weight: 12; length weight: 4.

Homologs are identified by any of a number of methods. By using probes, particularly labeled probes of DNA sequences, one can isolate homologous or related genes, as described in detail above. Briefly, a fragment of the provided cDNA can be used as a hybridization probe against a cDNA library from the target organism of interest, under various stringency conditions, e.g., low stringency conditions. The probe can be a large, fragment, or one or more short degenerate primers, and is typically labeled. Sequence identity can be determined by hybridization under stringent conditions, as described in detail above. Nucleic acids having a region of substantial identity or sequence similarity to the provided nucleic acid sequences, for example allelic variants, related genes, or genetically altered versions of the gene, bind to the provided sequences under less stringent hybridization conditions.

Alterations of the native amino acid sequence may be accomplished by any of a number of known techniques. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered gene having particular codons altered according to the substitution, deletion, or insertion required (Walder and Walder, 1986; Bauer et al., 1985; Craik, 1985; and U.S. Pat. Nos. 4,518,584 and 4,737,462)

Variants may comprise conservatively substituted sequences, meaning that one or more amino acid residues of a native polypeptide are replaced by different residues, but that the conservatively substituted polypeptide retains a desired biological activity that is essentially equivalent to that of a native polypeptide. Examples of conservative substitutions include substitution of amino acids that do not alter secondary and/or tertiary structure. Other examples involve substitution of amino acids outside the receptor-binding domain, when the desired biological activity is the ability to bind to a receptor on target cells. A given amino acid may be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Advantageously, the conserved amino acids are not altered when generating conservatively substituted sequences. If altered, amino acids found at equivalent positions in other members of the protein family, when known, are substituted.

In some embodiments, a subject polypeptide is present as an oligomer, including homodimers, homotrimers, homotetramers, and multimers that include more than four monomeric units. Oligomers also include heteromultimers, e.g., heterodimers, heterotrimers, heterotetramers, etc. where the subject polypeptide is present in a complex with proteins other than the subject polypeptide. Where the multimer is a heteromultimer, the subject polypeptide can be present in a 1:1 ratio, a 1:2 ratio, a 2:1 ratio, or other ratio, with the other protein(s).

Oligomers may be formed by disulfide bonds between cysteine residues on different polypeptides, or by non-covalent interactions between polypeptide chains, for example. In other embodiments, oligomers comprise from two to four polypeptides joined via covalent or non-covalent interactions between peptide moieties fused to the polypeptides. Such peptides may be peptide linkers (spacers), or peptides that have the property of promoting oligomerization. Leucine zippers and certain polypeptides derived from antibodies are among the peptides that can promote oligomerization of polypeptides attached thereto, as described in more detail below and in WO 94/10308.

Polypeptides of the invention can be obtained from naturally occurring sources or produced synthetically. The sources of naturally occurring polypeptides will generally depend on the species from which the protein is to be derived, i.e., the proteins will be derived from biological sources that express the proteins. The subject proteins can also be derived from synthetic means, e.g., by expressing a recombinant gene encoding a protein of interest in a suitable system or host or enhancing endogenous expression, as described in more detail below. Further, small peptides can be synthesized in the laboratory by techniques well known in the art.

The protein expression systems described below can produce fusion proteins that incorporate the polypeptides of the invention. The invention provides an isolated amino acid molecule with a first polypeptide comprising SEQ ID NOS.:7-8, 10-13, 17-18, and 20-27, or one or more of its biologically active fragments or variants, and a second molecule. This second molecule can facilitate production, secretion, and/or purification. It can confer a longer half-life to the first polypeptide when administered to an animal. Second molecules suitable for use in the invention include, e.g., polyethylene glycol (PEG), human serum albumin, fetuin, and/or one or more of their fragments as discussed below. The invention can also provide a nucleic acid molecule with a second nucleotide sequence that encodes a fusion partner. This second nucleotide sequence can be operably linked to the first nucleotide sequence.

Thus, the invention provides polypeptide fusion partners. They may be part of a fusion molecule, e.g., a polynucleotide or polypeptide, which represents the joining of all of or portions of more than one gene. For example, a fusion protein can be the product obtained by splicing strands of recombinant DNA and expressing the hybrid gene. A fusion molecule can be made by genetic engineering, e.g., by removing the stop codon from the DNA sequence of a first protein, then appending the DNA sequence of a second protein in frame. The DNA sequence will then be expressed by a cell as a single protein. Typically this is accomplished by cloning a cDNA into an expression vector in frame with an existing gene. The invention provides fusion proteins with heterologous and homologous leader sequences, fusion proteins with a heterologous amino acid sequence, and fusion proteins with or without N-terminal methionine residues. The fusion partners of the invention can be either N-terminal fusion partners or C-terminal fusion partners.

As noted above, suitable fusion partners include, but are not limited to, albumin and fetuin (U.S. Patent Application No. 60/589,788, which is herein incorporated by reference in its entirety). These fusion partners can include any variant of albumin, fetuin, or any fragment thereof. The natural fetuin polypeptides of the invention encompass all known isoforms and splice variants of fetuin A and B. The fetuin variants of the invention encompass any fetuin polypeptide with a high plasma half-life which is obtained by modification, such as by mutation, deletion, or addition. The invention encompasses all fetuin variants with a high plasma half-life obtained by in vitro modification of a polypeptide encoded by a fetuin polynucleotide. It includes non-natural sequences isolated from random peptide libraries. It also includes natural or artificial post-translational modifications, such as prenylation, glycosylation, e.g., with sialic acid, and the like. Modifications can be performed by any technique known in the art, such as commonly employed genetic engineering techniques. Such modified polypeptides can show, e.g., enhanced activity or increased stability. In addition, they may be purified in higher yields and show better solubility than the corresponding natural polypeptide, at least under certain purification and storage conditions.

Fusion polypeptides can be secreted from the cell by the incorporation of leader sequences that direct the protein to the membrane for secretion. These leader sequences can be specific to the host cell, and are known to skilled artisans; they are also cited in the references. The invention includes appropriate restriction enzyme sites for vector cloning. In addition to facilitating the secretion of these fusion proteins, the invention provides for facilitating their production. This can be accomplished in a number of ways, including producing multiple copies, employing strong promoters, and increasing their intracellular stability, e.g., by fusion with beta-galactosidase.

The invention also provides for facilitating the purification of these fusion proteins. Fusion with a selectable marker can facilitate purification by affinity chromatography. For example, fusion with the selectable marker glutathione S-transferase (GST) produces polypeptides that can be detected with antibodies directed against GST, and isolated by affinity chromatography on glutathione-sepharose; the GST marker can then be removed by thrombin cleavage. Polypeptides that provide for binding to metal ions are also suitable for affinity purification. For example, a fusion protein that incorporates Hisₙ, where n is between three and ten, inclusive, e.g., a 6×His-tag can be used to isolate a protein by affinity chromatography using a nickel ligand.

Suitable fusion partners that can be used to detect the fusion protein include all polypeptides that can bind to an antibody specific to the fusion partner (e.g., epitope tags, such as c-myc, hemagglutinin, and the FLAG® peptide, which is highly antigenic and provides an epitope reversibly bound by a specific monoclonal antibody, thus providing the fusion protein with a rapid assay and easy purification method); polypeptides that provide a detectable signal (e.g., a fluorescent protein, e.g., a green fluorescent protein, a fluorescent protein from an Anthozoan species; β-galactosidase; and luciferase). Also by way of example, where the fusion partner provides an immunologically recognizable epitope, an epitope-specific antibody can be used to quantitatively detect the level of polypeptide. In some embodiments, the fusion partner provides a detectable signal, and in these embodiments, the detection method is chosen based on the type of signal generated by the fusion partner. For example, where the fusion partner is a fluorescent protein, fluorescence is measured.

Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al., 2001; “humanized” recombinant GFP (hrGFP) (Stratagene); any of a variety of fluorescent and colored proteins from Anthozoan species, as described in, e.g., Matz et al., 1999.

Where the fusion partner is an enzyme that yields optically detectable product, the product can be detected using an appropriate means. For example, β-galactosidase can, depending on the substrate, yield a colored product that can detected with a spectrophotometer, and the protein luciferase can yield a luminescent product detectable with a luminometer.

The fusion partners of the invention can also include linkers, i.e., fragments of synthetic DNA containing a restriction endonuclease recognition site that can be used for splicing genes. These can include polylinkers, which contain several restriction enzyme recognition sites. A linker may be part of a cloning vector. It may be located either upstream or downstream of the therapeutic protein, and it may be located either upstream or downstream of the fusion partner.

Gene manipulation techniques have enabled the development and use of recombinant therapeutic proteins with fusion partners that impart desirable pharmacokinetic properties. Recombinant human serum albumin fused with synthetic heme protein has been reported to reversibly carry oxygen (Chuang et al., 2002). The long half-life and stability of human serum albumin (HSA) make it an attractive candidate for fusion to short-lived therapeutic proteins (U.S. Pat. No. 6,686,179).

For example, the short plasma half-life of unmodified interferon alpha makes frequent dosing necessary over an extended period of time, in order to treat viral and proliferative disorders. Interferon alpha fused with HSA has a longer half life and requires less frequent dosing than unmodified interferon alpha; the half-life was 18-fold longer and the clearance rate was approximately 140 times slower (Osborn et al., 2002). Interferon beta fused with HSA also has favorable pharmacokinetic properties; its half life was reported to be 36-40 hours, compared to 8 hours for unmodified interferon beta (Sung et al., 2003). A HSA-interleukin-2 fusion protein has been reported to have both a longer half-life and favorable biodistribution compared to unmodified interleukin-2. This fusion protein was observed to target tissues where lymphocytes reside to a greater extent than unmodified interleukin 2, suggesting that it exerts greater efficacy (Yao et al., 2004).

The Fc receptor of human immunoglobulin G subclass 1 has also been used as a fusion partner for a therapeutic molecule. It has been recombinantly linked to two soluble p75 tumor necrosis factor (TNF) receptor molecules. This fusion protein has been reported to have a longer circulating half-life than monomeric soluble receptors, and to inhibit TNFα-induced proinflammatory activity in the joints of patients with rheumatoid arthritis (Goldenberg, 1999). This fusion protein has been used clinically to treat rheumatoid arthritis, juvenile rheumatoid arthritis, psoriatic arthritis, and ankylosing spondylitis (Nanda and Bathon, 2004).

The peptides of the invention, including the fusion proteins, can be modified with or covalently coupled to one or more of a variety of hydrophilic polymers to increase their solubility and circulation half-life. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky 1995; Monfardini et al., 1995; U.S. Pat. Nos. 4,791,192; 4,670,417; 4,640,835; 4,496,689; 4,301,144; 4,179,337 and WO 95/34326.

Conjugating biomolecules with polyethylene glycol (PEG), a process known as pegylation, increases the circulating half-life of therapeutic proteins (Molineux, 2002). Polyethylene glycols are nontoxic water-soluble polymers that, owing to their large hydrodynamic volume, create a shield around the pegylated drug, thus protecting it from renal clearance, enzymatic degradation, and recognition by cells of the immune system.

Pegylated agents have improved pharmacokinetics that permit dosing schedules that are more convenient and more acceptable to patients. This improved pharmacokinetic profile may decrease adverse effects caused by the large variations in peak-to-trough plasma drug concentrations associated with frequent administration and by the immunogenicity of unmodified proteins (Harris et al., 2001). In addition, pegylated proteins may have reduced immunogenicity because PEG-induced steric hindrance can prevent immune recognition (Harris et al., 2001).

Polypeptides of the invention can be isolated by any appropriate means known in the art. For example, convenient protein purification procedures can be employed (e.g., Deuthscher et al., 1990). In general, a lysate can be prepared from the original source, (e.g., a cell expressing endogenous polypeptide, or a cell comprising the expression vector expressing the polypeptide(s)), and purified using HPLC, exclusion chromatography, gel electrophoresis, or affinity chromatography, and the like.

In another aspect, the invention provides a method of making a polypeptide of the invention by providing a nucleic acid molecule that Comprises a polynucleotide sequence encoding a polypeptide of the invention, introducing the nucleic acid molecule into an expression system, and allowing the polypeptide to be produced. Briefly, the methods generally involve introducing a nucleic acid construct into a host cell in vitro and culturing the host cell under conditions suitable for expression, then harvesting the polypeptide, either from the culture medium or from the host cell, (e.g., by disrupting the host cell), or both, as described in detail above. The invention also provides methods of producing a polypeptide using cell-free in vitro transcription/translation methods, which are well known in the art, also as provided above.

Specifically, the invention provides a method of making a polypeptide by providing a nucleic acid molecule that comprises a polynucleotide sequence encoding one or more isolated polypeptide comprising an amino acid sequence chosen from at least one amino acid sequence according to SEQ ID NOS.:7-8, 10-13, 17-18, and 20-27; introducing the nucleic acid molecule into an expression system; and allowing the polypeptide to be produced.

It also provides a method of making a polypeptide by providing a composition comprising a recombinant host cell that comprises at least one isolated nucleic acid molecule comprising at least one polynucleotide-sequence chosen from SEQ ID NOS.:1-2, 4-5, 31-32, and 35-36; sequences that hybridize to these sequences of under high stringency conditions; sequences having at least 80% sequence identity to the sequences of SEQ ID NOS.: 1-2, 4-5, 31-32, and 35-36 or sequences that hybridize to them under high stringency conditions; complements of any of these sequences; or biologically active fragments of any of the above-listed sequences; culturing the host cell to produce the polypeptide; and allowing the polypeptide to be produced.

Expression of the KIAA0779 Clones

The invention provides, as expression systems, any composition that permits protein synthesis when an expression vector is provided to the system. Expression systems are well-known by those skilled in the art. They include cell-free expression systems, e.g., wheat germ extract systems, rabbit reticulocyte lysate systems, and frog oocyte systems. They also include systems that utilize host cells, such as E. coli expression systems, yeast expression systems, insect expression systems, and mammalian expression systems, such as in CHO cells or 293 cells. The expression systems of the invention may also comprise translation systems, which support the processes by which the sequence of nucleotides in a messenger RNA molecule directs the incorporation of amino acids into a protein or polypeptide. Expression and translation systems of the invention may allow polypeptide synthesis, i.e., permit the incorporation of amino acids into a protein or polypeptide.

The invention provides both recombinant plasmid vectors and recombinant expression vectors. These recombinant vectors, or constructs, which can include nucleic acids of the invention, are useful for propagating a nucleic acid in a cell free expression system or host cell. Plasmid vectors can transfer nucleic acid between host cells derived from disparate organisms; these are known in the art as shuttle vectors. Plasmid vectors can also insert a subject nucleic acid into a host cell's chromosome; these are known in the art as insertion vectors.

Expression vectors of the invention are cloning vectors that contain regulatory sequences that allow transcription and translation of a cloned gene or genes and thus transcribe and clone DNA. They, can be used to express the polypeptides of the invention and typically include restriction sites to provide for the insertion of nucleic acid sequences encoding heterologous protein or RNA molecules.

Vectors can express either sense or antisense RNA transcripts of the invention in vitro (e.g., in a cell-free system or within an in vitro cultured host cell); these are known in the art as expression vectors. Expression vectors can also produce a subject polypeptide encoded by a subject nucleic acid. The expression vectors of the invention include both prokaryotic and eukaryotic expression vectors. The expression vectors of the invention provide a transcriptional and translational initiation region, which may be inducible or constitutive, where the coding region is under the transcriptional control of the transcriptional initiation region, and a transcriptional and translational termination region. These control regions can be native to a gene encoding the subject peptides, or can be derived from exogenous sources. Prior to vector insertion, a DNA of interest is obtained in a form substantially free of other nucleic acid sequences. The DNA can be recombinant, and flanked by one or more nucleotides with which it is not normally associated on a naturally occurring chromosome.

The expression vectors of the invention will generally have convenient restriction sites located near the promoter sequence to provide for the insertion of nucleic acid sequences encoding heterologous proteins. A selectable marker operative in the expression host can be present. Expression cassettes can be prepared comprising a transcription initiation region, the gene or fragment thereof, and a transcriptional termination region.

Expressed proteins and polypeptides can be obtained from naturally occurring sources or produced synthetically. For example, the proteins can be derived from biological sources that express the proteins. The proteins can also be derived synthetically, e.g., by expressing a recombinant gene encoding a protein of interest in a suitable host. Convenient protein purification procedures can be employed (Deutscher, 1990). For example, a lysate can be prepared from the original source, (e.g., a cell expressing endogenous polypeptide, or a cell comprising the expression vector expressing the polypeptide(s)), and purified using HPLC, exclusion chromatography, gel electrophoresis, or affinity chromatography.

Specifically, the invention provides a vector comprising an isolated nucleic acid molecule comprising at least one polynucleotide sequence chosen from SEQ ID NOS.: 1-2, 4-5, 31-32, and 35-36; sequences that hybridize to these sequences of under high stringency conditions; sequences having at least 80% sequence identity to the sequences of SEQ ID NOS.: 1-2, 4-5, 31-32, and 35-36 or sequences that hybridize to them under high stringency conditions; complements of any of these sequences; or biologically active fragments of any of the above-listed sequences. The invention also provides promoters of such vectors. Promoters of the invention can be inducible, and function as promoters upon receiving a predetermined stimulus; conditionally active, i.e., active only under defined circumstances, e.g., the cre-lox promoter; constitutive, i.e., active unless repressed; and/or tissue specific, i.e., initiate transcription exclusively or selectively in one or a few tissue types. Tissue-specific promoters of the invention include α-1 antitrypsin promoters, which are selective for lung tissue, albumin promoters, which are selective for hepatocytes, tyrosine hydrolase promoters, which are selective for melanocytes, villin promoters, which are selective for intestinal epithelium, glial fibrillary acidic protein promoters, which are selective for astrocytes, myelin basic protein promoters, which are selective for glial cells, and the immunoglobulin gene enhancer promoters, which are selective for B lymphocytes. Promoters of the invention vary in strength; promoter sequences at which RNA polymerase initiates transcription at a high frequency are classified as “strong,” and those with a low frequency of initiation as “weak.”

The invention includes DNA sequences that allow for the expression of biologically active fragments of the polypeptides of the invention. These include functional epitopes or domains, at least about 6 amino acids in length, at least about 12 amino acids in length, or at least about 25 amino acids in length, or any of the above-described fragments, up to and including the complete open reading frame of the gene. After introduction of these DNA sequences, the cells containing the construct can be selected by means of a selectable masker, and the selected cells expanded and used as expression-competent host cells, as described in further detail below.

Biologically active fragments include those with an improved desired activity, or a decreased undesirable activity. For example, an entity demonstrates biological activity when it participates in a molecular interaction with another molecule, or when it has therapeutic value in alleviating a disease condition, or when it has prophylactic value in inducing an immune response to the molecule, or when it has diagnostic value in determining the presence of the molecule. A biologically active polypeptide or fragment thereof includes one that can participate in a biological reaction, for example, as a transcription factor that combines with other transcription factors for initiation of transcription, or that can serve as an epitope or immunogen to stimulate an immune response, such as production of antibodies, or that can transport molecules into or out of cells, or that can perform a catalytic activity, for example polymerization or nuclease activity, or that can participate in signal transduction by binding to receptors, proteins, or nucleic acids, activating enzymes or substrates.

Cell-Free Expression Systems

Cell-free translation systems can be employed to produce proteins of the invention using RNAs derived from the DNA constructs of the present invention. Appropriate cloning and expression vectors, e.g., those containing SP6, or T7 promoters for use with prokaryotic and eukaryotic hosts, are known (Sambrook et al., 2001). These DNA constructs can be used to produce proteins in a rabbit reticulocyte lysate system, with wheat germ extracts, or with a frog oocyte system.

Expression in Host Cells

The invention provides a recombinant host cell comprising an isolated nucleic acid molecule comprising at least one polynucleotide sequence chosen from SEQ ID NOS.: 1-2, 4-5, 31-32, and 35-36; sequences that hybridize to these sequences of under high stringency conditions; sequences having at least 80% sequence identity to the sequences of SEQ ID NOS.: 1-2, 4-5, 31-32, and 35-36 or sequences that hybridize to them under high stringency conditions; complements of any of these sequences; or biologically active fragments of any of the above-listed sequences. Host cells of the invention include prokaryotic cells and eucaryotic cells. These host cells can be embodied as a human cell, a non-human mammalian cell, an insect cell, a fish cell, a plant cell, or a fungal cell.

Host cells of the invention include an individual cell, cell line, cell culture, or in vivo cell, which can be or has been a recipient of any polynucleotides or polypeptides of the invention, for example, a recombinant vector, an isolated polynucleotide, antibody, or fusion protein. Host cells include progeny of a single host cell; the progeny may not necessarily be completely identical (in morphology, physiology, or in total DNA, RNA, or polypeptide complement) to the original parent cell due to natural, accidental, or deliberate mutation and/or change. A host cell includes cells transformed, transfected, transduced, or infected in vivo or in vitro with a polynucleotide of the invention, for example, a recombinant vector. The invention provides recombinant host cells, which comprise a recombinant vector of the invention.

The invention also provides a method of making a recombinant host cell by providing a composition comprising a vector comprising an isolated nucleic acid molecule comprising at least one polynucleotide sequence chosen from SEQ ID NOS.:1-2, 4-5, 31-32, and 35-36; sequences that hybridize to these sequences of under high stringency conditions; sequences having at least 80% sequence identity to the sequences of SEQ ID NOS.:1-2, 4-5, 31-32, and 35-36 or sequences that hybridize to them under high stringency conditions; complements of any of these sequences; or biologically active fragments of any of the above-listed sequences and a promoter that regulates the expression of the nucleic acid molecule; and allowing a host cell to come into contact with the vector to form a recombinant host cell.

Host cells of the invention can express proteins and polypeptides in accordance with conventional methods, the method depending on the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g., COS 7 cells, can be used as the expression host cells. In some situations, it is desirable to express eukaryotic genes in eukaryotic cells, where the encoded protein will benefit from native folding and post-translational modifications.

When any of the above-referenced host cells, or other appropriate host cells or organisms, are used to duplicate and/or express the polynucleotides of the invention, the resulting duplicated nucleic acid, RNA, expressed protein, or polypeptide, is within the scope of the invention as a product of the host cell or organism. The product can be recovered by any appropriate means known in the art.

The sequence of a gene, including promoter regions and coding regions, can be mutated in various ways known in the art to generate targeted changes in promoter strength or in the sequence of the encoded protein. The DNA sequence or protein product of such a mutation will usually be substantially similar to the sequences provided herein, for example, will differ by at least one nucleotide or amino acid, respectively, and may differ by at least two nucleotides or amino acids. The sequence changes may be substitutions, insertions, deletions, or a combination thereof. Deletions may further include larger changes, such as deletions of a domain or exon. Other modifications of interest include epitope tagging, e.g., with the FLAG system or hemagglutinin.

Specific cellular expression systems of interest include plants, bacteria, yeast, insect cells and mammalian cell-derived expression systems. Representative systems from each of these categories are provided below.

Plants

Expression systems in plants include those described in U.S. Pat. No. 6,096,546 and U.S. Pat. No. 6,127,145.

Bacteria

Expression systems in bacteria include those described by Chang et al., 1978; Goeddel et al., 1979; Goeddel et al., 1980; EP 0 036,776; U.S. Pat. No. 4,551,433; DeBoer et al., 1983; and Siebenlist et al., 1980. For example, the expression vector pQE16 from the QIA expression prokaryotic protein expression system (Qiagen, Valencia, Calif.) can be used. The features of this vector that make it useful for protein expression include an efficient promoter (phage T5) to drive transcription, expression control provided by the lac operator system, which can be induced by addition of IPTG (isopropyl-beta-D-thiogalactopyranoside), and an encoded 6×His tag coding sequence. The latter is a stretch of six histidine amino acid residues which can bind very tightly to a nickel atom. This vector can be used to express a recombinant protein with a 6×His. tag fused to its carboxyl terminus, allowing rapid and efficient purification using Ni-coupled affinity columns. Any of the bacterial expression systems which are known in the art can be used in the invention.

Yeast

Expression systems in yeast include those described by Hinnen et al., 1978; Ito et al., 3983; Kurtz et al., 1986; Kunze et al., 1985; Gleeson et al., 1986; Roggenkamp et al., 1984; Das et al., 1984; De Louvencourt et al., 1983; Van den Berg et al., 1990; Kunze et al., 1985; Cregg et al., 1985; U.S. Pat. Nos. 4,837,148 and 4,929,555; Beach and Nurse, 1981; Davidow et al., 1987; Gaillardin et al., 1987; Ballance et al., 1983; Tilburn et al., 1983; Yelton et al., 1984; Kelly and Hynes, 1985; EP 0 244,234; WO 91/00357; and U.S. Pat. No. 6,080,559.

Insects

Expression systems for heterologous genes in insects includes those described in U.S. Pat. No. 4,745,051; Friesen et al., 1986; EP 0 127,839; EP 0 155,476; Vlak et al., 1988; Miller et al., 1988; Carbonell et al., 1988; Maeda et al., 1985; Lebacq-Verbeyden et al., 1988; Smith et al., 1985); Miyajima et al., 1987; and Martin et al., 1988. Numerous baculoviral strains and variants and corresponding permissive insect host cells are described in Luckow et al., 1988, Miller et al., 1988, and Maeda et al., 1985.

Mammals

Mammalian expression systems include those described in Dijkema et al., 1985; Gorman et al., 1982; Boshart et al., 1985; and U.S. Pat. No. 4,399,216. Additional features of mammalian expression are facilitated as described in Ham and McKeehan, 1979; Barnes and Sato, 1980 U.S. Pat. Nos. 4,767,704, 4,657,966, 4,927,762, 4,560,655, WO 90/103430, WO 87/00195, and U.S. RE 30,985.

A number of types of cells can act as suitable host cells for expression of the proteins. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

The polypeptides of the invention can be optimized for expression in each of the expression systems described above. For example, particular sequences can be introduced into the expression vector which optimize the expression of the protein in a yeast vector; other sequences can optimize the expression of the protein in a plant vector, and so forth. These sequences are known to skilled artisans and are described in the cited references.

Transgenic Animals

The polypeptides of the invention can also be expressed in animals, for example, transgenic animals. Animals of any species, including, but not limited to, mice, rats, rabbits, hamsters, guinea pigs, pigs, micro-pigs, goats, sheep, cows, and non-human primates, e.g., baboons, monkeys, and chimpanzees, may be used to generate transgenic animals. In a specific embodiment, techniques described herein or otherwise known in the art, are used to express polypeptides of the invention in humans, as part of a gene therapy protocol, as discussed in greater detail below.

Any technique known in the art may be used to introduce the transgene (i.e., polynucleotides of the invention) into animals to produce founder lines of transgenic animals. Such techniques include, but are not limited to, pronuclear microinjection (Paterson et al., 1994; Carver et al., 1993; Wright et al., 1991; and Hoppe et al., U.S. Pat. No. 4,873,191, 1989); retrovirus mediated gene transfer into germ lines (Van der Putten et al., 1985); blastocysts or embryos; gene targeting in embryonic stem cells (Thompson et al., 1989); electroporation of cells or embryos (Lo, 1983); introduction of the polynucleotides of the invention using a gene gun (se, e.g., Ulmer et al., 1993); introducing nucleic acid constructs into embryonic pluripotent stem cells and transferring the stem cells back into the blastocyst; and sperm-mediated gene transfer (Lavitrano et al., 1989). For a review of such techniques, see Gordon, 1989. See also, U.S. Pat. No. 5,464,764; U.S. Pat. No. 5,631,153; U.S. Pat. No. 4,736,866; and U.S. Pat. No. 4,873,191. Any technique known in the art may be used to produce transgenic clones containing polynucleotides of the invention, for example, nuclear transfer into enucleated oocytes of nuclei from cultured embryonic, fetal, or adult cells induced to quiescence (Campell et al., 1996; Wilmut et al., 1997).

The present invention provides for transgenic animals that carry the transgene in all their cells, as well as animals which carry the transgene in some, but not all their cells, i.e., mosaic animals or chimeras. The transgene may be integrated as a single transgene or as multiple copies, such as in concatamers, e.g., head-to-head tandem or head-to-tail tandem genes. The transgene may also be selectively introduced into and activated in a particular cell type by following, for example, the teaching of Lakso et al., (Lakso et al., 1992). The regulatory sequences required for such a cell-type, specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al., 1994. The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polynucleotides and polypeptides of the invention, studying conditions and/or disorders associated with aberrant expression, and in screening for compounds effective in ameliorating such conditions and/or disorders.

Accordingly, the invention provides an animal comprising one or more active agents chosen from an isolated nucleic acid molecule comprising at least one polynucleotide sequence chosen from SEQ ID NOS.:1-2, 4-5, 31-32, and 35-36; sequences that hybridize to these sequences of under high stringency conditions; sequences having at least 80% sequence identity to the sequences of SEQ ID NOS.: 1-2, 4-5, 31-32, and 35-36 or sequences that hybridize to them under high stringency conditions; complements of any of these sequences; or biologically active fragments of any of the above-listed sequences; a vector comprising such a nucleic acid molecule and a promoter that regulates the expression of the nucleic acid molecule; a recombinant host cell that comprises such a nucleic acid molecule; an isolated polypeptide comprising an amino acid sequence, wherein the amino acid sequence is chosen from SEQ ID NOS.:7-8, 10-13, 17-18, and 20-27, or a biologically active fragment thereof, or is encoded by a polynucleotide sequence chosen from SEQ ID NOS.:1-2, 4-5, 31-32, and 35-36, or a biologically active fragment thereof; and an antibody that specifically binds to and/or interferes with the biological activity of such a nucleic acid molecule or a polypeptide or one or more of any of their biologically active fragments.

Diagnostic Kits and Methods

The invention provides a kit comprising one or more of a polynucleotide, polypeptide, or modulator composition, such as an antibody composition, which may include instructions for its use. Such kits are useful in diagnostic applications, for example, to detect the presence and/or level of a polypeptide in a biological sample by specific antibody interaction. Specifically, the invention provides a diagnostic kit comprising an isolated nucleic acid molecule that comprises a sequence of at least 6, at least 7, at least 8, or at least 9 contiguous nucleotides chosen from SEQ ID NOS.: 1-2, 4-5, 31-32, and 35-36; sequences that hybridize to these sequences of under high stringency conditions; sequences having at least 80% sequence identity to the sequences of SEQ ID NOS.: 1-2, 4-5, 31-32, and 35-36 or sequences that hybridize to them under high stringency conditions; complements of any of these sequences; or biologically active fragments of any of the above-listed sequences.

In an embodiment, the invention comprises a diagnostic kit comprising a polypeptide molecule which comprises an amino acid sequence chosen from SEQ ID NOS.:7-30 or a biologically active fragment thereof, or is encoded by a polynucleotide sequence chosen from SEQ ID NOS.:1-6, 31-38, and reagents to carry out an immunoassay. The invention also comprises a diagnostic kit comprising an antibody specifically binding to and/or interfering with the biological activity of an isolated nucleic acid molecule comprising at least one polynucleotide sequence chosen from SEQ ID NOS.:1-6, 31-38; sequences that hybridize to these sequences of under high stringency conditions; sequences having at least 80% sequence identity to the sequences of SEQ ID NOS.:1-6, 31-38 or sequences that hybridize to them under high stringency conditions; complements of any of these sequences; or biologically active fragments of any of the above-listed sequences, or an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, 31-38, or a biologically active fragment thereof; and reagents to carry out an immunoassay. The kit may also comprise instructions for its use.

A kit, or pharmaceutical pack, of the invention can comprise one or more containers filled with one or more of the ingredients of the pharmaceutical compositions of the invention, as described in more detail below. Associated with such container(s) can be a notice in the form prescribed by a governmental agency regulating the manufacture, use, or sale of pharmaceuticals or biological products, which notice reflects approval by the agency of manufacture, use, or sale for human administration.

Kits that detect a polynucleotide can comprise a moiety that specifically hybridizes to a polynucleotide of the invention. The primer nucleic acids can be prepared using any known method, e.g., automated synthesis. In some embodiments, one or both members of the pair of nucleic acid molecules comprise a detectable label. Kits of the invention for detecting a subject polypeptide will comprise a moiety that specifically binds to a polypeptide of the invention; the moiety includes, but is not limited to, a polypeptide-specific antibody.

Kits for detecting polynucleotides can also comprise a pair of nucleic acids in a suitable storage medium, e.g., a buffered solution, in a suitable container. The pair of isolated nucleic acid molecules serve as primers in an amplification reaction, e.g., a polymerase chain reaction. The kit can further include additional buffers, reagents for polymerase chain reaction, e.g., deoxynucleotide triphosphates (dNTP), a thermostable DNA polymerase, a solution containing Mg²⁺ ions, e.g., MgCl₂, and other components well known to those skilled in the art for carrying out a polymerase chain reaction. The kit can further include instructions for use, which may be provided in a variety of forms, e.g., printed information, or compact disc. The kit may further include reagents necessary to extract DNA from a biological sample and reagents for generating a cDNA copy of an mRNA. The kit may optionally provide additional useful components, including, but not limited to, buffers, developing reagents, labels, reacting surfaces, means for detections, control samples, standards, and interpretive information.

The kits of the invention can detect one or more molecules of the invention present in biological samples, including biological fluids such as blood, serum, plasma, urine, cerebrospinal fluid, tears, saliva, lymph, dialysis fluid, lavage fluid, semen, and other liquid samples of biological origin. A biological sample can include cells and their progeny, including cells in situ, cells ex vivo, cells in culture, cell supernatants, and cell lysates. It ran include organ or tissue culture derived fluids, tissue biopsy samples, tumor biopsy samples, stool samples, and fluids extracted from cells and tissues. Cells dissociated from solid tissues, tissue sections, and cell lysates are also included. A biological sample can comprise a sample that has been manipulated after its procurement, such as by treatment with reagents, solubilization, or enrichment for certain components, such as polynucleotides or polypeptides. Biological samples suitable for use in the kit also include derivatives and fractions of biological samples.

The kits are useful in diagnostic applications. For example, the kit is useful to determine whether a given DNA sample isolated from an individual comprises an expressed nucleic acid, a polymorphism, or other variant. The kit can be used to detect a specific disorder or disease, i.e., a pathological, abnormal, and/or harmful condition which can be identified by symptoms or other identifying factors as diverging from a healthy or a normal state.

The present invention provides methods for detecting the presence and/or biological activity of a subject polypeptide in a biological sample. The assay used will be appropriate to the biological activity of the particular polypeptide. Thus, e.g., where the biological activity is an enzymatic activity, the method will involve contacting the sample with an appropriate substrate, and detecting the product of the enzymatic reaction on the substrate. Where the biological activity is binding to a second macromolecule, the assay detects protein-protein binding, protein-DNA binding, protein-carbohydrate binding, or protein-lipid binding, as appropriate, using well known assays. Where the biological activity is signal transduction (e.g., transmission of a signal from outside the cell to inside the cell) or transport, an appropriate assay is used, such as measurement of intracellular calcium ion concentration, measurement of membrane conductance changes, or measurement of intracellular potassium ion concentration.

The invention provides a method of determining the presence of or measuring the level of an isolated nucleic acid molecule comprising at least one polynucleotide sequence chosen from SEQ ID NOS.:1-2, 4-5, 31-32, and 35-36; sequences that hybridize to these sequences of under high stringency conditions; sequences having at least 80% sequence identity to the sequences of SEQ ID NOS.:1-2, 4-5, 31-32, and 35-36 or sequences that hybridize to them under high stringency conditions; complements of any of these sequences; or biologically active fragments of any of the above-listed sequences by providing a complement of the nucleic acid molecule or providing a complement to the complement of the nucleic acid molecule; allowing the molecule to interact with the sample; and determining whether interaction has occurred.

The invention also provides a method of determining the presence of or measuring the level of an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-2, 4-5, 31-32, and 35-36, or a biologically active fragment thereof by providing an antibody that specifically binds to or interferes with the activity of the polypeptide; allowing the antibody to interact with the polypeptide in the sample, if any; and determining whether interaction has occurred.

The invention further provides methods for detecting the presence or measuring the level of a normal or abnormal polypeptide in a biological sample using a specific antibody. The methods generally comprise contacting the sample with a specific antibody and detecting binding between the antibody and molecules of the sample. Specific antibody binding, when compared to a suitable control, is an indication that a polypeptide of interest is present in the sample. Suitable controls include a sample known not to contain the polypeptide, and a sample contacted with a non-specific antibody, e.g., an anti-idiotype antibody. Specifically, the invention provides a method of determining the presence of or measuring the level of a specific antibody to a polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-2, 4-5, 31-32, and 35-36, or a biologically active fragment thereof by providing such a polypeptide; allowing the polypeptide to interact with a specific antibody in the sample, if present; and determining whether interaction has occurred.

A variety of methods to detect specific antibody-antigen interactions are known in the art, e.g., standard immunohistological methods, immunoprecipitation, enzyme immunoassay, and radioimmunoassay. The specific antibody can be detectably labeled, either directly or indirectly, as described at length herein, and cells are permeabilized to stain cytoplasmic molecules. Briefly, antibodies are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, specific-binding pairs may be used, involving, e.g., a second stage antibody or reagent that is detectably-labeled, as described above. Such reagents and their methods of use are well known in the art.

Alternatively, a biological sample can be brought into contact with an immobilized antibody on a solid support or carrier, such as nitrocellulose, that is capable of immobilizing cells, cell particles, or soluble proteins. The antibody can be attached (coupled) to an insoluble support, such as a polystyrene plate or a bead. After contacting the sample, the support can then be washed with suitable buffers, followed by contacting with a detectably-labeled specific antibody. Detection methods are known in the art and will be chosen as appropriate to the signal emitted by the detectable label. Detection is generally accomplished in comparison to suitable controls, and to appropriate standards.

The invention provides a method of diagnosing cancer, or a proliferative, inflammatory, immune, viral, bacterial, or metabolic disorder in a patient, by allowing an antibody specific for a polypeptide of the invention to contact a patient sample, and detecting specific binding between the antibody and any antigen in the sample to determine whether the subject has cancer, proliferative, inflammatory, immune, viral, bacterial, or metabolic disorder.

The invention further provides a method of diagnosing cancer, or a proliferative, inflammatory, immune, viral, bacterial, or metabolic disorder in a patient, by allowing a polypeptide of the invention 4 to contact a patient sample, and detecting specific binding between the polypeptide and any interacting molecule in the sample to determine whether the subject has cancer, proliferative, inflammatory, immune, viral, bacterial, or metabolic disorder.

The invention provides diagnostic kits and methods for diagnosing disease states based on the detected presence, amount, and/or biological activity of polynucleotides or polypeptides in a biological sample. These detection methods can be provided as part of a kit which detects the presence amount, and/or biological activity of a polynucleotide or polypeptide in a biological sample. Procedures using these kits can be performed by clinical laboratories, experimental laboratories, medical practitioners, or private individuals.

Diagnostic methods in which the level of expression is of interest will typically involve determining whether a specific nucleic acid or amino acid molecule is present, and/or comparing its abundance in a sample of interest with that of a control value to determine any relative differences. These differences can then be measured qualitatively and/or quantitatively, and differences related to the presence or absence of an abnormal expression pattern. A variety of different methods for determining the presence or absence of a nucleic acid or polypeptide in a biological sample are known to those of skill in the art; particular methods of interest include those described by Soares, 1997; Pietu et al., 1996; Stolz and Tuan, 1996; Zhao et al., 1995; Chalifour et al., 1994; Raval, 1994; McGraw, 1984; and Hong, 1982. Also of interest are the methods disclosed in WO 97/27317.

Where the kit provides for mRNA detection, detection of hybridization, when compared to a suitable control, is an indication of the presence in the sample of a subject polynucleotide. Appropriate controls include, for example, a sample which is known not to contain subject polynucleotide mRNA, and use of a labeled polynucleotide of the same “sense” as a subject polynucleotide mRNA. Conditions which allow hybridization are known in the art and described in greater detail above. Detection can be accomplished by any known method, including, but not limited to, in situ hybridization, PCR, RT-PCR, and “Northern” or RNA blotting, or combinations of such techniques, using a suitably labeled subject polynucleotide. Specific hybridization can be determined by comparison to appropriate controls.

Where the kit provides for polypeptide detection, it can include one or more specific antibodies. In some embodiments, the antibody specific to the polypeptide is detectably labeled. In other embodiments, the antibody specific to the polypeptide is not labeled; instead, a second, detectably-labeled antibody is provided that binds to the specific antibody. The kit may further include blocking reagents, buffers, and reagents for developing and/or detecting the detectable marker. The kit may further include instructions for use, controls, and interpretive information.

Detection of specific binding of an antibody, when compared to a suitable control, is an indication that a subject polypeptide is present in the sample. Suitable controls include a sample known not to contain a subject polypeptide; and a sample contacted with an antibody not specific for the subject polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. These methods are known to those skilled in the art (Harlow et al., 1998; Harlow and Lane, 1988).

Where the kit provides for specific antibody detection, it can include one or more polypeptides. In some embodiments, the polypeptide is detectably labeled. In other embodiments, the polypeptide is not labeled; instead, a detectably-labeled ligand or second antibody is provided that specifically binds to the polypeptide. The kit may further include blocking reagents, buffers, and reagents for developing and/or detecting the detectable marker. The kit may further include instructions for use, controls, and interpretive information.

The invention further provides for kits with unit doses of an active agent. These agents are described in more detail below. In some embodiments, the agent is provided in oral or injectable doses. Such kits can comprise a receptacle containing the unit doses and an informational package insert describing the use and attendant benefits of the drugs in treating a condition of interest.

The present invention provides methods for diagnosing disease states based on the detected presence and/or level of polynucleotide or polypeptide in a biological sample, and/or the detected presence and/or level of biological activity of the polynucleotide or polypeptide. These detection methods can be provided as part of a kit. Thus, the invention further provides kits for detecting the presence and/or a level of a polynucleotide or polypeptide in a biological sample and/or or the detected presence and/or level of biological activity of the polynucleotide or polypeptide. Procedures using these kits can be performed by clinical laboratories, experimental laboratories, medical practitioners, or private individuals.

Therapeutic Compositions and Methods

Therapeutic Compositions

Certain of the proteins of the present invention are useful for diagnosis, prophylaxis, or treatment of disorders in subjects that are deficient in such proteins or require regeneration of certain tissues, the proliferation of which is dependent on such proteins, or requires an inhibition or activation of growth that is dependent on such proteins.

The proteins of the invention can be screened for functional activities in appropriate functional assays, as is conventional in the art. Such assays include, for example, in vitro and in vivo assays for factors that stimulate the proliferation or differentiation of stem cells, progenitor cells, or precursor cells into T cells, B cells, pancreatic islet cells, bone cells, neuronal cells, etc.

The use of SEQ ID NOS.:1-38 has therapeutic applications for the diseases and disorders discussed above. Compositions based on these sequences, biologically active fragments, and variants thereof, can be formulated using well-known reagents and methods, and can be provided in formulation with pharmaceutically acceptable excipients, a wide variety of which are known in the art (Gennaro, 2003). Therapeutic compounds comprising these sequences can be formulated into preparations in solid, semi-solid, liquid, or gaseous forms, such as tablets, capsules, powders, granules, ointments, solutions, suppositories, injections, inhalants, and aerosols.

The invention provides a composition comprising a pharmaceutically acceptable carrier or excipient and one or more active agents chosen from an isolated nucleic acid molecule comprising at least one polynucleotide sequence chosen from SEQ ID NOS.:1-6, and 31-38; sequences that hybridize to these sequences of under high stringency conditions; sequences having at least 80% sequence identity to the sequences of SEQ ID NOS.:1-6, and 31-38 or sequences that hybridize to them under high stringency conditions; complements of any of these sequences; or biologically active fragments of any of the above-listed sequences; a vector comprising such a nucleic acid molecule and a promoter that regulates its expression; an isolated polypeptide comprising an amino acid sequence; wherein the amino acid sequence is chosen from SEQ ID NOS.:7-30, or a biologically active fragment thereof, or is encoded by a polynucleotide sequence chosen from SEQ ID NOS.:1-6, and 31-38, or a biologically active fragment thereof; and an antibody that specifically binds to or interferes with the activity of such a polypeptide.

Pharmaceutically acceptable carriers of the invention, also known as pharmaceutically acceptable diluents, pharmaceutically acceptable excipients, or pharmaceutically acceptable vehicles, include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Adjuvants of the invention include, but are not limited to Freunds's, Montanide ISA Adjuvants [Seppic, Paris, France], Ribi's Adjuvants (Ribi ImmunoChem Research, Inc., Hamilton, Mont.), Hunter's TiterMax (CytRx Corp., Norcross, Ga.), Aluminum Salt Adjuvants (Alhydrogel-Superfos of Denmark/Accurate Chemical and Scientific Co., Westbury, N.Y.), Nitrocellulose-Adsorbed Protein, Encapsulated Antigens, and Gerbu Adjuvant (Gerbu Biotechnik GmbH, Gaiberg, Germany/C-C Biotech, Poway, Calif.). Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers such as Azone can also be included.

Pharmaceutically acceptable salts used in carrying out the invention include the acid addition salts (formed with the free amino groups of the polypeptide) and which are formed with inorganic acids such as, for example, hydrochloric or phosphoric acids, or such organic acids as acetic, mandelic, oxalic, and tartaric. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, and histidine.

Compositions for oral administration can form solutions, suspensions, tablets, pills, capsules, sustained release formulations, oral rinses, or powders. These compositions can be provided in unit dosage forms, i.e., physically discrete units suitable as unitary dosages for human and animal subjects, each unit containing a predetermined quantity of compounds of the present invention calculated in an effective amount, that is, a dosage sufficient to produce the desired result or effect in association with a pharmaceutically acceptable carrier. The specifications for the novel unit dosage forms of the present invention depend on the particular compound employed, the host, and the effect to be achieved, as well as the pharmacodynamics associated with each compound in the host.

Typically, such a composition will contain from less than 1% to about 95% of the active ingredient, preferably about 1.0% to about 50%. Generally, between about 100 mg and 500 mg will be administered to a child and between about 500 mg and 5 grams will be administered to an adult. Administration is generally by injection and often by injection to a localized area. Administration may be performed by stereotactic injection. The frequency of administration will be determined by the care giver based on patient responsiveness. Other effective dosages can be readily determined by one of ordinary skill in the art through routine trials establishing dose response curves.

In order to calculate the effective amount of subject polynucleotide or polypeptide agent, those skilled in the art could use readily available information, with respect to the amount of agent necessary to have a the desired effect. The amount of an agent necessary to increase a level of active subject polynucleotide or polypeptide can be calculated from in vitro experimentation. The amount of agent will, of course, vary depending upon the particular agent used.

Other effective dosages can be readily determined by one of ordinary skill in the art through routine trials establishing dose response curves, for example, the amount of agent necessary to increase a level of active subject polypeptide can be calculated from in vitro experimentation. Those of skill will readily appreciate that dose levels can vary as a function of the specific compound, the severity of the symptoms, and the susceptibility of the subject to side effects, and preferred dosages for a given compound are readily determinable by those of skill in the art by a variety of means. For example, in order to calculate the dose, those skilled in the art can use readily available information with respect to the amount necessary to have the desired effect, depending upon the particular agent used.

Therapeutic and Related Methods

Identifying Interactive Biological Molecules

The present polynucleotides, polypeptides, and modulators find use in therapeutic agent screening/discovery applications, such as screening for receptors or competitive ligands, for use, for example, as small molecule therapeutic drugs. Also provided are methods of modulating a biological activity of a polypeptide and methods of treating associated disease conditions, particularly by administering modulators of the present polypeptides, such as small molecule modulators, antisense molecules, and specific antibodies.

Formation of a binding complex between a subject polypeptide and an interacting polypeptide or other macromolecule (e.g., DNA, RNA, lipids, polysaccharides, and the like) can be detected using any known method. Suitable methods include: a yeast two-hybrid system (Zhu et al., 1997; Fields and Song, 1989; U.S. Pat. No. 5,283,173; Chien et al., 1991); a mammalian cell two-hybrid method; a fluorescence resonance energy transfer (FRET) assay; a bioluminescence resonance energy transfer (BRET) assay; a fluorescence quenching assay; a fluorescence anisotropy assay (Jameson and Sawyer, 1995); an immunological assay; and an assay involving binding of a detectably labeled protein to an immobilized protein.

Detection methods of the invention can be qualitative or quantitative. Thus, as used herein, the terms detection, identification, determination, and the like, refer to both qualitative and quantitative determinations, and include “measuring.” For example, detection methods include methods for detecting the presence and/or level of polynucleotide or polypeptide in a biological sample, and methods for detecting the presence and/or level of biological activity of polynucleotide or polypeptide in a sample.

Immunological assays, and assays involving binding of a detectably labeled protein to an immobilized protein can be performed in a variety of ways. For example, immunoprecipitation assays can be designed such that the complex of protein and an interacting polypeptide is detected by precipitation with an antibody specific for either the protein or the interacting polypeptide.

Detecting mRNA Levels and Monitoring Gene Expression

The present invention provides methods for detecting the presence of mRNA in a biological sample. The methods can be used, for example, to assess whether a test compound affects gene expression, either directly or indirectly. The present invention provides diagnostic methods to compare the abundance of a nucleic acid with that of a control value, either qualitatively or quantitatively, and to relate the value to a normal or abnormal expression pattern.

Methods of measuring mRNA levels are known in the art (Pietu, 1996; Zhao, 1995; Soares, 1997; Raval, 1994; Chalifour, 1994; Stolz, 1996; Hong, 1982; McGraw, 1984; WO 97/27317). These methods generally comprise contacting a sample with a polynucleotide of the invention under conditions that allow hybridization and detecting hybridization, if any, as an indication of the presence of the polynucleotide of interest. Appropriate controls include the use of a sample lacking the polynucleotide mRNA of interest, or the use of a labeled polynucleotide of the same “sense” as a polynucleotide mRNA of interest. Detection can be accomplished by any known method, including, but not limited to, in situ hybridization, PCR, RT-PCR, and “Northern” or RNA blotting, or combinations of such techniques, using a suitably labeled subject polynucleotide. A variety of labels and labeling methods for polynucleotides are known in the art and can be used in the assay methods of the invention. A common method employed is use of microarrays which can be purchased or customized, for example, through conventional vendors such as Affymetrix.

In some embodiments, the methods involve generating a cDNA copy of an mRNA molecule in a biological sample, and amplifying the cDNA using an isolated primer pairs as described above, i.e., a set of two nucleic acid molecules that serve as forward and reverse primers in an amplification reaction (e.g., a polymerase chain reaction). The primer pairs are chosen to specifically amplify a cDNA copy of an mRNA encoding a polypeptide. A detectable label can be included in the amplification reaction, as provided above. Methods using PCR amplification can be performed on the DNA from a single cell, although it is convenient to use at least about 10⁵ cells.

The present invention provides methods for monitoring gene expression. Changes in a promoter or enhancer sequence that can affect gene expression can be examined in light of expression levels of the normal allele by various methods known in the art. Methods for determining promoter or enhancer strength include quantifying the expressed natural protein, and inserting the variant control element into a vector with a quantitative reporter gene such as β-galactosidase, luciferase, or chloramphenicol acetyltransferase (CAT).

Detecting Polymorphisms and Mutations

Biochemical studies can determine whether a sequence polymorphism in a coding region or control region is associated with disease. Disease-associated polymorphisms can include deletion or truncation of the gene, mutations that alter expression level, or mutations that affect protein function, etc. A number of methods are available to analyze nucleic acids for the presence of a specific sequence, e.g., a disease associated polymorphism. Genomic DNA can be used when large amounts of DNA are available. Alternatively, the region of interest is cloned into a suitable vector and grown in sufficient quantity for analysis. Cells that express the gene provide a source of mRNA, which can be assayed directly or reverse transcribed into cDNA for analysis. The nucleic acid can be amplified by conventional techniques, i.e., PCR, to provide sufficient amounts for analysis. (Saiki et al., 1988; Sambrook et al., 1989, pp. 14.2-14.33). Alternatively, various methods are known in the art that utilize oligonucleotide ligation as a means of detecting polymorphisms (Riley et al., 1990; Delahunty et al., 1996).

The sample nucleic acid, e.g., an amplified or cloned fragment, is analyzed by one of a number of methods known in the art. The nucleic acid can be sequenced by dideoxy nucleotide sequencing, or other methods, and the sequence of bases compared to a wild-type sequence. Hybridization with the variant sequence can also be used to determine its presence, e.g., by Southern blots, dot blots, etc. The hybridization pattern of a control and variant sequence to an array of oligonucleotide probes immobilized on a solid support, as described in U.S. Pat. No. 5,445,934, or WO 95/35505, can also be used as a means of detecting the presence of variant sequences. Single strand conformational polymorphism (SSCP) analysis, denaturing gradient gel electrophoresis (DGGE), and heteroduplex analysis in gel matrices can detect variation as alterations in electrophoretic mobility resulting from conformational changes created by DNA sequence alterations. Alternatively, where a polymorphism creates or destroys a recognition site for a restriction endonuclease, the sample can be digested with that endonuclease, and the products fractionated according to their size to determine whether the fragment was digested. Fractionation can be performed by gel or capillary electrophoresis, for example with acrylamide or agarose gels.

Screening for mutations in a gene can be based on the functional or antigenic characteristics of the protein. Protein truncation assays are useful in detecting deletions that might affect the biological activity of the protein. Various immunoassays designed to detect polymorphisms in proteins can be used in screening. Where many diverse genetic mutations lead to a particular disease phenotype, functional protein assays have proven to be effective screening tools. The activity of the encoded protein can be determined by comparison with the wild-type protein.

The present invention further features a method of identifying an agent that modulates the level of a subject polypeptide (or an mRNA encoding a subject polypeptide) in a cell. The method generally involves contacting a cell (e.g., a eukaryotic cell) that produces the subject polypeptide with a test agent; and determining the effect, if any, of the test agent on the level of the polypeptide in the cell.

The present invention further features a method of identifying an agent that modulates biological activity of a subject polypeptide. The methods generally involve contacting a subject polypeptide with a test agent; and determining the effect, if any, of the test agent on the activity of the polypeptide. In certain embodiments, a polypeptide is expressed on a cell surface. In certain embodiments, the agent or modulator is an antibody, for example, where an antibody binds to the polypeptide or affects its biological activity. In other embodiments, the agent or modulator is an inhibitory RNA molecule. The present invention further features biologically active agents (or modulators) identified using a method of the invention.

The present invention also features a method of modulating biological activity using an agent selectable by the above methods. Generally, methods of the invention can encompass modulating biological activity by contacting an agent with a first human or a non-human host cell, thereby modulating the activity of the first host cell or a second host cell. In one example, contacting the agent with the first human or non-human host cell results in the recruitment of a second host cell. The agent may, as described in more detail below, be an antibody or antibody fragment of the invention.

The modulation can comprise directly enhancing cell activity, indirectly enhancing cell activity, directly inhibiting cell activity, or indirectly inhibiting cell activity. The cell activity that is modulated can include transcription, translation, cell cycle control, signal transduction, intracellular trafficking, cell adhesion, cell mobility, proteolysis, cell growth, differentiation, and/or activities corresponding to the predicted function of the cDNA clone of the invention, as described in the Tables and throughout the specification. The modulation can result in cell death or apoptosis, or inhibition of cell death or apoptosis, as well as cell growth, cell proliferation, or cell survival, or inhibition of cell growth, cell proliferation, or cell survival.

Either the first or the second host cell can be a human or a non-human host cell. Either the first or the second host cell can be an immune cell, e.g., a T cell, B cell, NK cell, dendritic cell, macrophage, muscle cell, stem cell, skin cell, fat cell, blood cell, brain cell, bone marrow cell, endothelial cell, retinal cell, bone cell, kidney cell, pancreatic cell, liver cell, spleen cell, prostate cell, cervical cell, ovarian cell, breast cell, lung cell, liver cell, soft tissue cell, colorectal cell, other cell of the gastrointestinal tract, or a cancer cell.

The invention provides a method of modulating cell growth, differentiation, function, or other activity in an animal in need of such modulation by administering a composition with a therapeutically effective amount of a modulator, e.g., a polypeptide with the amino acid sequence of SEQ. ID. NOS.:7-30 or one or more active fragment or variant thereof, a polypeptide encoded by SEQ. ID. NOS.:1-6, 31-38, or one or more active fragment or variant thereof, or an agonist or antagonist thereof. The cell growth, differentiation, function, or activity can be associated with cancer or other proliferative or inflammatory disorders.

The therapeutic compositions can be administered in a variety of ways. These include oral, buccal, rectal, parenteral, including intranasal, intramuscular, intravenous, intra-arterial, intraperitoneal, intradermal, transdermal, subcutaneous, intratracheal, intracardiac, intraventricular, intracranial, intrathecal, etc., and administration by implantation. The agents may be administered daily, weekly, or monthly, as appropriate as conventionally determined.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules, or capsules, for example, with conventional additives, such as lactose, mannitol, corn starch or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art (Gennaro, 2003). The composition or formulation to be administered will, in any event, contain a quantity of the polypeptide adequate to achieve the desired state in the subject being treated.

Antisense RNA, siRNA, and Peptide Aptamers

In an embodiment of the invention, antisense reagents can be used to down-regulate gene expression (Phillips, 1999a; Phillips, 1999b). The antisense reagent can be one or more antisense oligonucleotide, particularly synthetic antisense oligonucleotides with chemical modifications of native nucleic acids, or nucleic acid constructs that express antisense molecules, e.g., RNA based on one or more nucleotide sequences found in the Sequence Listing. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNAse H, or by steric hindrance. One or a combination of antisense molecules can be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule (U.S. Patent Application No. 60/589,806, which are herein incorporated by reference in their entireties). Alternatively, the antisense molecule is a synthetic oligonucleotide. Antisense oligonucleotides will generally be at least about 7, usually at least about 12, more usually at least about 20 nucleotides in length, usually not more than about 35 nucleotides in length, and usually not more than about 50, and not more than about 500, where the length is governed by efficiency of inhibition, specificity, including absence of cross-reactivity, and the like. Short oligonucleotides, of from 7 to 8 bases in length, can be strong and selective inhibitors of gene expression (Wagner et al., 1996).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides can be chemically synthesized by methods known in the art (Wagner et al. 1993; Milligan et al., 1993). Preferred oligonucleotides are chemically modified from the native phosphodiester structure, in order to increase their intracellular stability and binding affinity. A number of such modifications have been described in the literature, which modifications alter the chemistry of the backbone, sugars or heterocyclic bases.

As an alternative to antisense inhibitors, catalytic nucleic acid compounds, e.g., ribozymes, antisense conjugates, interfering RNA, etc. can be used to inhibit gene expression. Ribozymes can be synthesized in vitro and administered to the patient, or encoded in an expression vector, from which the ribozyme is synthesized in the targeted cell (WO 95/23225; Beigelman et al., 1995). Examples of oligonucleotides with catalytic activity are described in WO 95/06764. Conjugates of anti-sense ODN with a metal complex, e.g., terpyridylCu(II), capable of mediating mRNA hydrolysis are described in Bashkin et al., 1995.

Small interfering RNA (siRNA) can also be used as an inhibitor. Small interfering RNA can also be used to screen for biologically active agents by administering siRNA compositions to cells, monitoring for a change in a readable biological activity, and repeating the administration and monitoring with a subset of the plurality of siRNA compositions to determine which silenced gene is responsible for the change, then identifying the transcriptional or translational gene product of the silenced gene. The transcriptional or translational product so identified may represent a biologically active agent, responsible for the change which is determined by the readable biological activity.

The invention provides methods of producing libraries of siRNA molecules by enzymatically engineering DNA, including generating siRNAs by intra-molecular sense- and antisense single-stranded DNA ligation. Libraries of siRNA molecules can also be produced by two converging, opposing RNA polymerase III promoters (Kaykas and Moon, 2004; Zhang and Williams, U.S. patent application for Small Interfering RNA Libraries, 2004). The resulting siRNA can selectively inhibit gene expression relevant to a specific cell, tissue, protein family, or disease (Zhang and Williams, U.S. patent application for Small Interfering RNA Libraries, 2004).

Small interfering RNA compositions, including the libraries of the invention, can be used to screen populations of transfected cells for phenotypic changes. Cells with the desired phenotype can be recovered, and the siRNA construct can be characterized. The screening can be performed using oligonucleotides specific to any open reading frame, including enzymatically fragmented, open reading frames, e.g., with restriction endonucleases. The screening can also be performed using random siRNA libraries, including enzymatically fragmented libraries, e.g., with restriction endonucleases.

The invention provides a method of using siRNA to identify one or more specific siRNA molecules effective against one or more polypeptides of the invention or fragments thereof. This method can be performed by administering the composition to cells expressing the mRNA, monitoring for a change in a readable biological activity, e.g., activity relevant to a disease condition, and repeating the administration and monitoring with a subset of a plurality of siRNA molecules, thereby identifying one or more specific siRNA molecules effective against one or more genes relevant to a disease condition.

This method includes using one or more siRNA molecules so identified for treating or preventing a disease, by administering the identified siRNA to patient in an amount effective to inhibit one or more genes relevant to the disease. This method can be performed, e.g., by gene therapy, described in more detail below, by administering an effective amount of the identified specifier siRNA to a patient. This method can also be performed by administering an effective amount of the identified specific siRNA to a patient by administering a nucleic acid vaccine, either with or without an adjuvant, also described in more detail below. The siRNA molecules and compositions of the invention can be also used in diagnosing a given disease or abnormal condition by administering any of the siRNA molecules or compositions of the invention to a biological sample and monitoring for a change in a readable biological activity to identify the disease or abnormal condition.

Another suitable agent for reducing an activity of a subject polypeptide is a peptide aptamer. Peptide aptamers are peptides or small polypeptides that act as dominant inhibitors of protein function; they specifically bind to target proteins, blocking their function (Kolonin et al., 1998). Due to the highly selective nature of peptide aptamers, they may be used not only to target a specific protein, but also to target specific functions of a given protein (e.g., a signaling function). Further, peptide aptamers may be expressed in a controlled fashion by use of promoters which regulate expression in a temporal, spatial or inducible manner. Peptide aptamers act dominantly; therefore, they can be used to analyze proteins for which loss-of-function mutants are not available.

Modulating the Expression of cDNA Clones

The invention provides screening methods for identifying agents that modulate the level of a mRNA molecule of the invention, agents that modulate the level of a polypeptide of the invention, and agents that modulate the biological activity of a polypeptide of the invention. In some embodiments, the assay is cell-free; in others, it is cell-based. Where the screening assay is a binding assay, one or more of the molecules can be joined to a label, where the label can directly or indirectly provide a detectable signal.

In these embodiments, the candidate agent is combined with a cell possessing a polynucleotide transcriptional regulatory element operably linked to a polypeptide-coding sequence of interest, e.g., a subject cDNA or its genomic component; and determining the agent's effect on polynucleotide expression, as measured, for example by the level of mRNA, polypeptide, or fusion polypeptide.

In other embodiments, for example, a recombinant vector can comprise an isolated polynucleotide transcriptional regulatory sequence, such as a promoter sequence, operably linked to a reporter gene (e.g., β-galactosidase, CAT, luciferase, or other gene that can be easily assayed for expression). In these embodiments, the method for identifying an agent that modulates a level of expression of a polynucleotide in a cell comprises combining a candidate agent with a cell comprising a transcriptional regulatory element operably linked to a reporter gene; and determining the effect of said agent on reporter gene expression.

Known methods of measuring mRNA levels can be used to identify agents that modulate mRNA levels, including, but not limited to, PCR with detectably-labeled primers. Similarly, agents that modulate polypeptide levels can be identified using standard methods for determining polypeptide levels, including, but not limited to an immunoassay such as ELISA with detectably-labeled antibodies.

A wide variety of cell-based assays can also be used to identify agents that modulate eukaryotic or prokaryotic mRNA and/or polypeptide levels. Examples include transformed cells that over-express a cDNA construct and cells transformed with a polynucleotide of interest associated with an endogenously-associated promoter operably linked to a reporter gene. A control sample would comprise, for example, the same cell lacking the candidate agent. Expression levels are measured and compared in the test and control samples.

The cells used in the assay are usually mammalian cells, including, but not limited to, rodent cells and human cells. The cells can be primary cell cultures or can be immortalized cell lines. Cell-based assays generally comprise the steps of contacting the cell with a test agent, forming a test sample, and, after a suitable time, assessing the agent's effect on macromolecule expression. That is, the mammalian cell line is transformed or transfected with a construct that results in expression of the polynucleotide, the cell is contacted with a test agent, and then mRNA or polypeptide levels are detected and measured using conventional assays.

A suitable period of time for contacting the agent with the cell can be determined empirically, and is generally a time sufficient to allow entry of the agent into the cell and to allow the agent to have a measurable effect on subject mRNA and/or polypeptide levels. Generally, a suitable time is between about 10 minutes and about 24 hours, including about 1 to about 8 hours. Alternatively, incubation periods may be between about 0.1 and about 1 hour, selected for example for optimum activity or to facilitate rapid high-throughput screening. Where the polypeptide is expressed on the cell surface, however, a shorter length of time may be sufficient. Incubations are performed at any suitable temperature, i.e., between about 4° C. and about 40° C. The contact and incubation steps can be followed by a washing step to remove unbound components, i.e., a label that would give rise to a background signal during subsequent detection of specifically-bound complexes.

A variety of assay configurations and protocols are known in the art. For example, one of the components can be bound to a solid support, and the remaining components contacted with the support bound component Remaining components may be added at different times or at substantially the same time. Further, where the interacting protein is a second subject protein, the effect of the test agent on binding can be determined by determining the effect on multimization of the subject protein.

The invention provides a method of identifying an agent that modulates the biological activity of an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, and 31-38, or a biologically active fragment thereof by providing such a polypeptide; allowing at least one modulating agent to contact the polypeptide; and selecting an agent that modulates the biological activity of the polypeptide. The invention also provides a modulator composition comprising a modulator and a pharmaceutically acceptable carrier, wherein the modulator is obtainable by this method. The modulator can, e.g., be an antibody or a small molecule drug.

The method generally comprises contacting a test agent with a sample containing a subject polypeptide and assaying a biological activity of the subject polypeptide in the presence of the test agent. An increase or a decrease in the assayed biological activity in comparison to the activity in a suitable control (e.g., a sample comprising a subject polypeptide in the absence of the test agent) is an indication that the substance modulates a biological activity of the subject polypeptide. The mixture of components is added in any order that provides for the requisite interaction.

Some embodiments will detect agents that decrease the biological activity of a molecule of the invention. Maximal inhibition of the activity is not always necessary, or even desired, in every instance to achieve a therapeutic effect. Agents that decrease a biological activity can find use in treating disorders associated with the biological activity of the molecule. Alternatively, some embodiments will detect agents that increase a biological activity. Agents that increase a biological activity of a molecule of the invention can find use in treating disorders associated with a deficiency in the biological activity. Agents that increase or decrease a biological activity of a molecule of the invention can be selected for further study, and assessed for physiological attributes, i.e., cellular availability, cytotoxicity, or biocompatibility, and optimized as required. For example, a candidate agent is assessed for any cytotoxic activity it may exhibit toward the cell used in the assay using well-known assays, such as trypan blue dye exclusion, an MTT ([3-(4,5-dimethylthiazol-2-yl)-2,5-diphenyl-2H-tetrazolium bromide]) assay, and the like.

A variety of other reagents can be included in the screening assay. These include salts, neutral proteins, e.g., albumin, detergents, and other compounds that facilitate optimal binding and/or reduce non-specific or background interactions. Reagents that improve the efficiency of the assay, such as protease inhibitors, nuclease inhibitors, or anti-microbial agents, etc., can be used.

Candidate modulating agents of the invention encompass numerous chemical classes, typically synthetic, semi-synthetic, or naturally occurring inorganic or organic molecules, small molecules, or macromolecular complexes. Candidate agents can be small organic compounds having a molecular weight of more than about 50 and less than about 2,500 daltons. Candidate agents can comprise functional groups necessary for structural interaction with proteins, particularly hydrogen bonding, and can include at least an amine, carbonyl, hydroxyl or carboxyl group, and can contain at least two of the functional chemical groups. The candidate agents can comprise cyclical carbon or heterocyclic structures and/or aromatic or polyaromatic structures substituted with one or more of the above functional groups. Candidate agents are also found among biomolecules, including oligonucleotides, polynucleotides, and fragments thereof, depsipeptides, polypeptides and fragments thereof, oligosaccharides, polysaccharides and fragments thereof, lipids, fatty acids, steroids, purines, pyrimidines, derivatives thereof, structural analogs, modified nucleic acids, modified, derivatized or designer amino acids, or combinations thereof. An agent which modulates a biological activity of a subject polypeptide may, e.g., increase or decrease the activity at least about 10%, at least about 15%, at least about 20%, at least about 25%, at least about 50%, at least about 100%, or at least about 2-fold, at least about 5-fold, or at least about 10-fold or more when compared to a suitable control.

Candidate agents are obtained from a wide variety of sources including libraries of synthetic or natural compounds. Numerous means are available for random and directed synthesis of a wide variety of organic compounds and biomolecules, including expression of randomized oligonucleotides and oligopeptides. For example, random peptide libraries obtained by yeast two-hybrid screens (Xu et al., 1997), phage libraries (Hoogenboom et al., 1998), or chemically generated libraries. Alternatively, libraries of natural compounds in the form of bacterial, fungal, plant and animal extracts are available or readily produced, including antibodies produced upon immunization of an animal with subject polypeptides, or fragments thereof, or with the encoding polynucleotides. Additionally, natural or synthetically produced libraries and compounds are readily modified through conventional chemical, physical and biochemical means, and can be used to produce combinatorial libraries. Further, known pharmacological agents can be subjected to directed or random chemical modifications, such as acylation, alkylation, esterification, and amidification, etc, to produce structural analogs.

Modulating agents can include agonists and antagonists. Agonist refers herein to a substance that mimics the function of an active molecule. Agonists include, but are not limited to, drugs, hormones, antibodies, and neurotransmitters, as well as analogues and fragments thereof. Antagonist herein refers to a molecule that competes for the binding sites of an agonist, but does not induce an active response. Antagonists include, but are not limited to, drugs, hormones, antibodies, and neurotransmitters, as well as analogues and fragments thereof.

In one embodiment of the invention, complementary sense and antisense RNAs derived from a substantial portion of the subject polynucleotide are synthesized in vitro. The resulting sense and antisense RNAs are annealed in an injection buffer, and the double-stranded RNA injected or otherwise introduced into the subject, i.e., in food or by immersion in buffer containing the RNA (Gaudilliere et al., 2002; O'Neil et al., 2001; WO99/32619). In another embodiment, dsRNA derived from a gene of the present invention is generated in vivo by simultaneously expressing both sense and antisense RNA from appropriately positioned promoters operably linked to coding sequences in both sense and antisense orientations.

Antibodies

The invention provides an antibody directed to a polypeptide encoded by a nucleic acid molecule with SEQ ID NO.:1-6. The invention also provides an antibody specifically binding to and/or interfering with the biological activity of an isolated nucleic acid molecule comprising at least one polynucleotide sequence chosen from SEQ ID NOS.:1-6, and 31-38; sequences that hybridize to these sequences of under high stringency conditions; sequences having at least 80% sequence identity to the sequences of SEQ ID NOS.:1-6, and 31-38 or sequences that hybridize to them under high stringency conditions; complements of any of these sequences; or biologically active fragments of any of the above-listed sequences; or an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, and 31-38, or a biologically active fragment thereof.

This antibody may also be a monoclonal antibody, a polyclonal antibody, a single chain antibody, an Fab fragment, an antibody comprising a backbone of a molecule with an Ig domain, a V_{H} fragment, a V_{L} fragment, a cdr fragment, and/or a framework fragment. It may also be a cytotoxic antibody, targeting antibody, an antibody agonist, an antibody antagonist, an antibody that promotes endocytosis of a target antigen, an antibody that mediates ADCC, and/or an antibody that mediates CDC.

An antibody of the invention can be a human antibody, a non-human primate antibody, a non-primate animal antibody, a rabbit antibody, a mouse antibody, a rat antibody, a sheep antibody, a goat antibody, a horse antibody, a porcine antibody, a cow antibody, a chicken antibody, a humanized antibody, a primatized antibody, and a chimeric antibody. These antibodies can comprise a cytotoxic antibody with one or more cytotoxic component chosen from a radioisotope, a microbial toxin, a plant toxin, and a chemical compound. The chemical compound can be chosen from doxorubicin and cisplatin.

In another aspect, the invention provides antibody targets. The polynucleotides and polypeptides of the invention comprise nucleic acid and amino acid sequences that can be recognized by antibodies. A target sequence can be any polynucleotide or amino acid sequence of approximately eighteen or more contiguous nucleotides or six or more amino acids. A variety of comparing means can be used to accomplish comparison of sequence information from a sample (e.g., to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art. A target sequence includes an antibody target sequence, which refers to an amino acid sequence that can be used as an immunogen for injection into animals for production of antibodies or for screening against a phage display or antibody library for identification of binding partners.

The invention provides target structural motifs, or target motifs, i.e., any rationally selected sequences or combination of sequences in which the sequence(s) are chosen based on a three-dimensional configuration formed upon the folding of the target motif, or on consensus sequences of regulatory or active sites. There are a variety of target motifs known in the art. Protein target motifs include, but are not limited to, enzyme active sites and signal sequences. Nucleic acid target motifs include, but are not limited to, hairpin structures, promoter sequences, and other expression elements, such as binding sites for transcription factors.

Antibodies of the invention bind specifically to their targets. The term binds specifically, in the context of antibody binding, refers to high avidity and/or high affinity binding of an antibody to a specific polypeptide, or more accurately, to an epitope of a specific polypeptide. Antibody binding to such epitope on a polypeptide can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide, which does not comprise the epitope. Antibodies that bind specifically to a subject polypeptide may be capable of binding other polypeptides at a weak, yet detectable, level (e.g., 10%, or less of the binding shown to the polypeptide of interest). Such weak binding, or background binding, is readily discernible from the specific antibody binding to a subject polypeptide, e.g., by use of appropriate controls. In general, antibodies of the invention bind to a specific polypeptide with a binding affinity of 10⁻⁷ M or greater (e.g., 10⁻⁸ M, 10⁻⁹ M, 10⁻¹⁰, 10⁻¹¹, etc.).

The invention provides antibodies that can distinguish the variant sequences of the invention from currently known sequences. These antibodies can distinguish polypeptides that differ by no more than one amino acid (U.S. Pat. No. 6,656,467). They have high affinity constants, i.e., in the range of approximately 10⁻¹⁰M, and are produced, for example, by genetically engineering appropriate antibody gene sequences, according to the method described by Young et al., in U.S. Pat. No. 6,656,467.

Antibodies of the invention can be provided as matrices, i.e., as geometric networks of antibody molecules and their antigens, as found in immunoprecipitation and flocculation reactions. An antibody matrix can exist in solution or on a solid phase support.

Antibodies of the invention can be provided as a library of antibodies or fragments thereof, wherein at least one antibody or fragment thereof specifically binds to at least a portion of a polypeptide comprising an amino acid sequence according to SEQ ID NOS.:7-30, and/or wherein at least one antibody or fragment thereof interferes with at least one activity of such polypeptide or fragment thereof. In certain embodiments, the antibody library comprises at least one antibody or fragment thereof that specifically inhibits binding of a subject polypeptide to its ligand or substrate, or that specifically inhibits binding of a subject polypeptide as a substrate to another molecule. The present invention also features corresponding polynucleotide libraries comprising at least one polynucleotide sequence that encodes an antibody or antibody fragment of the invention. In specific embodiments, the library is provided on a nucleic acid array or in computer-readable format.

The invention provides a method of making an antibody by introducing an antigen chosen from an isolated nucleic acid molecule comprising at least one polynucleotide sequence chosen from SEQ ID NOS.:1-6, 31-38; sequences that hybridize to these sequences of under high stringency conditions; sequences having at least 80% sequence identity to the sequences of SEQ ID NOS.: 1-6, 31-38 or sequences that hybridize to them under high stringency conditions; complements of any of these sequences; or biologically active fragments of any of the above-listed sequences or an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, 31-38, or a biologically active fragment thereof into an animal in an amount sufficient to elicit generation of antibodies specific to the antigen, and recovering the antibodies therefrom.

Generally, the invention features a method of making an antibody by immunizing a host animal (Coligan, 2002). In this method, a polypeptide or a fragment thereof, a polynucleotide encoding a polypeptide, or a polynucleotide encoding a fragment thereof, is introduced into an animal in a sufficient amount to elicit the generation of antibodies specific to the polypeptide or fragment thereof, and the resulting antibodies are recovered from the animal. Initial immunizations can be performed using either polynucleotides or polypeptides. Subsequent booster immunizations can also be performed with either polynucleotides or polypeptides. Initial immunization with a polynucleotide can be followed with either polynucleotide or polypeptide immunizations, and an initial immunization with a polypeptide can be followed with either polynucleotide or polypeptide immunizations.

The host animal will generally be a different species than the immunogen, e.g., a human protein used to immunize mice. Methods of antibody production are well known in the art (Coligan, 2002; Howard and Bethell, 2000; Harlow et al., 1998; Harlow and Lane, 1988). The invention thus also provides a non-human animal comprising an antibody of the invention. The animal can be a non-human primate, (e.g., a monkey) a rodent (e.g., a rat, a mouse, a hamster, a guinea pig), a chicken, cattle (e.g., a sheep, a goat, a horse, a pig, a cow), a rabbit, a cat, or a dog.

The present invention also features a method of making an antibody by isolating a spleen from an animal injected with a polypeptide or a fragment thereof, a polynucleotide encoding a polypeptide, or a polynucleotide encoding a fragment thereof, and recovering antibodies from the spleen cells. Hybridomas can be made from the spleen cells, and hybridomas secreting specific antibodies can be selected.

The present invention further features a method of making a polynucleotide library from spleen cells, and selecting a cDNA clone that produces specific antibodies, or fragments thereof. The cDNA clone or a fragment thereof can be expressed in an expression system that allows production of the antibody or a fragment thereof, as provided herein.

The immunogen can comprise a nucleic acid, a complete protein, or fragments and derivatives thereof, or proteins expressed on cell surfaces. Proteins domains, e.g., extracellular, cytoplasmic, or luminal domains can be used as immunogens. Immunogens comprise all or a part of one of the subject proteins, where these amino acids contain post-translational modifications, such as glycosylation, found on the native target protein. Immunogens comprising protein extracellular domains are produced in a variety of ways known in the art, e.g., expression of cloned genes using conventional recombinant methods, or isolation from tumor cell culture supernatants, etc. The immunogen can also be expressed in vivo from a polynucleotide encoding the immunogenic peptide introduced into the host animal.

Polyclonal antibodies are prepared by conventional techniques. These include immunizing the host animal in vivo with the target protein for immunogen) in substantially pure form, for example, comprising less than about 1% contaminant. The immunogen can comprise the complete target protein, fragments, or derivatives thereof. To increase the immune response of the host animal, the target protein can be combined with an adjuvant; suitable adjuvants include alum, dextran, sulfate, large polymeric anions, and oil & water emulsions, e.g., Freund's adjuvant (complete or incomplete). The target protein can also be conjugated to synthetic carrier proteins or synthetic antigens. The target protein is administered to the host, usually intradermally, with an initial dosage followed by one or more, usually at least two, additional booster dosages. Following immunization, blood from the host is collected, followed by separation of the serum from blood cells. The immunoglobulin present in the resultant antiserum can be further fractionated using known methods, such as ammonium salt fractionation, or DEAE chromatography and the like.

Cytokines can also be used to help stimulate immune response. Cytokines act as chemical messengers, recruiting immune cells that help the killer T-cells to the site of attack. An example of a cytokine is granulocyte-macrophage colony-stimulating factor (GM-CSF), which stimulates the proliferation of antigen-presenting cells, thus boosting an organism's response to a cancer vaccine. As with adjuvants, cytokines can be used in conjunction with the antibodies and vaccines disclosed herein. For example, they can be incorporated into the antigen-encoding plasmid or introduced via a separate plasmid, and in some embodiments, a viral vector can be engineered to display cytokines on its surface.

The method of producing polyclonal antibodies can be varied in some embodiments of the present invention. For example, instead of using a single substantially isolated polypeptide as an immunogen, one may inject a number of different immunogens into one animal for simultaneous production of a variety of antibodies. In addition to protein immunogens, the immunogens can be nucleic acids (e.g., in the form of plasmids or vectors) that encode the proteins, with facilitating agents, such as liposomes, microspheres, etc, or without such agents, such as “naked” DNA.

The invention provides a bacteriophage comprising an antibody specifically binding to and/or interfering with the biological activity of an isolated nucleic acid molecule comprising at least one polynucleotide sequence chosen from SEQ ID NOS.:1-6, and 31-38; sequences that hybridize to these sequences of under high stringency conditions; sequences having at least 80% sequence identity to the sequences of SEQ ID NOS.:1-6, and 31-38 or sequences that hybridize to them under high stringency conditions; complements of any of these sequences; or biologically active fragments of any of the above-listed sequences; or an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, and 31-38, or a biologically active fragment thereof; or a fragment of such an antibody. The invention further provides a bacterial cell comprising such a bacteriophage. It provides a recombinant host cell that produces such an antibody or a fragment of such an antibody.

In an embodiment, polyclonal antibodies can be prepared using phage display libraries, which are conventional in the art. In this method, a collection of bacteriophages displaying antibody properties on their surfaces are made to contact subject polypeptides, or fragments thereof. Bacteriophages displaying antibody properties that specifically recognize the subject polypeptides are selected, amplified, for example, in E. coli, and harvested. Such a method typically produces single chain antibodies, which are further described below.

Phage display technology can be used to produce Fab antibody fragments, which can be then screened to select those with strong and/or specific binding to the protein targets. The screening can be performed using methods that are known to those of skill in the art, for example, ELISA, immunoblotting, immunohistochemistry, or immunoprecipitation. Fab fragments identified in this manner can be assembled with an Fc portion of an antibody molecule to form a complete immunoglobulin molecule.

Monoclonal antibodies are also produced by conventional techniques, such as fusing an antibody-producing plasma cell with an immortal cell to produce hybridomas. Suitable animals will be used, e.g., to raise antibodies against a mouse polypeptide of the invention, the host animal will generally be a hamster, guinea pig, goat, chicken, or rabbit, and the like. Generally, the spleen and/or lymph nodes of an immunized host animal provide the source of plasma cells, which are immortalized by fusion with myeloma cells to produce hybridoma cells. Culture supernatants from individual hybridomas are screened using standard techniques to identify clones producing antibodies with the desired specificity. The antibody can be purified from the hybridoma cell supernatants or from ascites fluid present in the host by conventional techniques, e.g., affinity chromatography using antigen, e.g., the subject protein, bound to an insoluble support, i.e., protein A sepharose, etc.

The antibody can be produced as a single chain, instead of the normal multimeric structure of the immunoglobulin molecule. Single chain antibodies have been previously described (i.e., Jost et al., 1994). DNA sequences encoding parts of the immunoglobulin, for example, the variable region of the heavy chain and the variable region of the light chain are ligated to a spacer, such as one encoding at least about four small neutral amino acids, i.e., glycine or serine. The protein encoded by this fusion allows the assembly of a functional variable region that retains the specificity and affinity of the original antibody.

The invention also provides intrabodies that are intracellularly expressed single-chain antibody molecules designed to specifically bind and inactivate target molecules inside cells. Intrabodies have been used in cell assays and in whole organisms (Chen et al., 1994; Hassanzadeh et al., 1998). Inducible expression vectors can be constructed with intrabodies that react specifically with a protein of the invention. These vectors can be introduced into host cells and model organisms.

The invention also provides “artificial” antibodies, e.g., antibodies and antibody fragments produced and selected in vitro. In some embodiments, these antibodies are displayed on the surface of a bacteriophage or other viral particle, as described above. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected for example, on the basis of phage binding to selected antigens, can be fused to ac fragment of an immunoglobulin for use as a therapeutic, as described, for example, in U.S. Pat. No. 5,116,964 or WO 99/61630. Antibodies of the invention can be used to modulate biological activity of cells, either directly or indirectly. A subject antibody can modulate the activity of a target cell, with which it has primary interaction, or it can modulate the activity of other cells by exerting secondary effects, i.e., when the primary targets interact or communicate with other cells. The antibodies of the invention can be administered to mammals, and the present invention includes such administration, particularly for therapeutic and/or diagnostic purposes in humans.

The antibodies can be partially human or fully human antibodies. For example, xenogenic antibodies, which are produced in animals that are transgenic for human antibody genes, can be employed to make a fully human antibody. By xenogenic human antibodies is meant antibodies that are fully human antibodies, with the exception that they are produced in a non-human host that has been genetically engineered to express human antibodies (e.g., WO 98/50433; WO 98/24893 and WO 99/53049).

Chimeric immunoglobulin genes constructed with immunoglobulin cDNA are known in the art (Liu et al. 1987a; Liu et al. 1987b). Messenger RNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant (c) regions genes are known in the art (Kabat et al., 1991). Human C region genes are readily available from known clones. The choice of isotype will be guided by the desired effector functions, such as complement fixation, or antibody-dependent cellular cytotoxicity. IgG1, IgG3 and IgG4 isotypes, and either of the kappa or lambda human light chain constant regions can be used. The chimeric, humanized antibody is then expressed by conventional methods.

Consensus sequences of heavy (H) and light (L) J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

A convenient expression vector for producing antibodies is one that encodes a functionally complete human CH or CL immunoglobulin sequence, with appropriate restriction sites engineered so that any VH or VL sequence can be easily inserted and expressed, such as plasmids, retroviruses, YACs, or EBV derived episomes, and the like. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama, et al. 1983), Rous sarcoma virus LTR (Gorman et al. 1982), and Moloney murine leukemia virus LTR (Grosschedl et al. 1985), or native immunoglobulin promoters.

Antibody fragments, such as Fv, F(ab′)₂, and Fab can be prepared by cleavage of the intact protein, e.g., by protease or chemical cleavage. These fragments can include heavy and light chain variable regions. Alternatively, a truncated gene can be designed, e.g., a chimeric gene encoding a portion of the F(ab′)₂ fragment that includes DNA sequences encoding the CH1 domain and hinge region of the H chain, followed by a translational stop codon.

Antibodies may be administered by injection systemically, such as by intravenous injection; or by injection or application to the relevant sire, such as by direct injection into a tumor, or direct application to the site when the site is exposed in surgery; or by topical application, such as if the disorder is on the skin, for example.

For in vivo use, particularly for injection into humans, in some embodiments it is desirable to decrease the antigenicity of the antibody. An immune response of a recipient against the antibody may potentially decrease the period of time that the therapy is effective. Methods of humanizing antibodies are known in the art. The humanized antibody can be the product of an animal having transgenic human immunoglobulin genes, e.g., constant region genes (e.g., Grosveld and Kolias, 1992; Murphy and Carter, 1993; Pinkert, 1994; and International Patent Applications WO 90/10077 and WO 90/04036). Alternatively, the antibody of interest can be engineered by recombinant DNA techniques to substitute the CH1, CH2, CH3, hinge domains, and/or the framework domain with the corresponding human sequence (see, e.g., WO 92/02190). Humanized antibodies can also be produced by immunizing mice that make human antibodies, such as Abgenix xenomice, Medarex's mice, or Kirin's mice, and can be made using the technology of Protein Design Labs, Inc. (Fremont, Calif.) (Coligan, 2002). Both polyclonal and monoclonal antibodies made in non-human animals may be humanized before administration to human subjects.

The antibodies of the present invention may be administered alone or in combination with other molecules for use as a therapeutic, for example, by linking the antibody to cytotoxic agent or radioactive molecule. Radioactive antibodies that are specific to a cancer cell, disease cell, or virus-infected cell may be able to deliver a sufficient dose of radioactivity to kill such cancer cell, disease cell, or virus-infected cell. The antibodies of the present invention can also be used in assays for detection of the subject polypeptides. In some embodiments, the assay is a binding assay that detects binding of a polypeptide with an antibody specific for the polypeptide; the subject polypeptide or antibody can be immobilized, while the subject polypeptide and/or antibody can be detectably-labeled. For example, the antibody can be directly labeled or detected with a labeled secondary antibody. That is, suitable, detectable labels for antibodies include direct labels, which label the antibody to the protein of interest, and indirect labels, which label an antibody that recognizes the antibody to the protein of interest.

These labels include radioisotopes, including, but not limited to ⁶⁴Cu, ⁶⁷Cu, ⁹⁰Y, ¹²⁴I, ¹²⁵I, ¹³¹I, ¹³⁷Cs, ¹⁸⁶Re, ²¹¹At, ²¹²Bi, ²¹³Bi, ²²³Ra, ²⁴¹Am, and ²⁴⁴Cm; enzymes having detectable products (e.g., luciferase, β-galactosidase, and the like); fluorescers and fluorescent labels, e.g., as provided herein; fluorescence emitting metals, e.g., ¹⁵²Eu, or others of the lanthanide series, attached to the antibody through metal chelating groups such as EDTA; chemiluminescent compounds, e.g., luminol, isoluminol, or acridinium salts; and bioluminescent compounds, e.g., luciferin, or aequorin (green fluorescent protein), specific binding molecules, e.g., magnetic particles, microspheres, nanospheres, and the like.

Alternatively, specific-binding pairs may be used, involving, e.g., a second stage antibody or reagent that is detectably-labeled and that an amplify the signal. For example, a primary antibody can be conjugated to biotin, and horseradish peroxidase-conjugated strepavidin added as a second stage reagent. Digoxin and antidigoxin provide another such pair. In other embodiments, the secondary antibody can be conjugated to an enzyme such as peroxidase in combination with a substrate that undergoes a color change in the presence of the peroxidase. The absence or presence of antibody binding can be determined by various methods, including flow cytometry of dissociated cells, microscopy, radiography, or scintillation counting. Such reagents and their methods of use are well known in the art.

Nucleic acid, polypeptides, and antibodies of the invention can be provided in the form of arrays. The term “array” or “microarray” is used interchangeably and refers to a collection of plural biological molecules such as nucleic acids, polypeptides, or antibodies, having locatable addresses that may be separately detectable. Generally, a microarray encompasses use of sub microgram quantities of biological molecules. The biological molecules may be affixed to a substrate or may be in solution or suspension. The substrate can be porous or solid, planar or non-planar, unitary or distributed, such as a glass slide, a 96 well plate, with or without the use of microbeads or nanobeads. As such, the term “microarray” includes all of the devices referred to as microarrays in Schena, 1999; Bassett et al., 1999; Bowtell, 1999; Brown and Botstein, 1999; Chakravarti, 1999; Cheung et al., 1999; Cole et al., 1999; Collins, 1999; Debouck and Goodfellow, 1999; Duggan et al., 1999; Hacia, 1999; Lander, 1999; Lipshutz et al., 1999; Southern, et al., 1999; Schena, 2000; Brenner et al., 2000; Lander, 2001; Steinhaur et al., 2002; and Espejo et al, 2002. Nucleic acid microarrays include both oligonucleotide arrays (DNA chips) containing expressed sequence tags (“ESTs”) and arrays of larger DNA sequences representing a plurality of genes bound to the substrate, either one of which can be used for hybridization studies. Protein and antibody microarrays include arrays of polypeptides or proteins, including but not limited to, polypeptides or proteins obtained by purification, fusion proteins, and antibodies, and can be used for specific binding studies (Zhu and Snyder, 2003; Houseman et al., 2002; Schaeferling et al., 2002; Weng et al., 2002; Winssinger et al., 2002; Zhu et al., 2001; Zhu et al. 2001; and MacBeath and Schreiber, 2000).

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines.

Treating Cancer and Proliferative Conditions

The invention provides a method of treating a disease, disorder, syndrome, or condition in a subject by administering an composition comprising an antibody specifically binding to and/or interfering with the biological activity of an isolated nucleic acid molecule comprising at least one polynucleotide sequence chosen from SEQ ID NOS.:1-6, and 31-38; sequences that hybridize to these sequences of under high stringency conditions; sequences having at least 80% sequence identity to the sequences of SEQ ID NOS.:1-6, and 31-38 or sequences that hybridize to them under high stringency conditions; complements of any of these sequences; or biologically active fragments of any of the above-listed sequences; or an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, and 31-38, or a biologically active fragment thereof; or a fragment of such an antibody; and a pharmaceutically acceptable carrier or excipient; to a subject. This method can be used to treat a proliferative, inflammatory, immune related, or metabolic disease, disorder, syndrome, or condition.

The therapeutic compositions and methods of the invention can be used in the treatment of cancer, i.e., an abnormal malignant cell or tissue growth, e.g., a tumor. In an embodiment, the compositions and methods of the invention kill tumor cells. In an embodiment, they inhibit tumor development. Cancer is characterized by the proliferation of abnormal cells that tend to invade the surrounding tissue and metastasize to new body sites. The growth of cancer cells exceeds that of and is uncoordinated with the normal cells and tissues. In an embodiment, the compositions and methods of the invention inhibit the progression of premalignant lesions to malignant tumors.

Cancer encompasses carcinomas, which are cancers of epithelial cells, and are the most common forms of human cancer; carcinomas include squamous cell carcinoma, adenocarcinoma, melanomas, and hepatomas. Cancer also encompasses sarcomas, which are tumors of mesenchymal origin, and includes osteogenic sarcomas, leukemias, and lymphomas. Cancers can have one or more than one neoplastic cell type. Some characteristics that can, in some instances, apply to cancer cells are that they are morphologically different from normal cells, and may appear anaplastic; they have a decreased sensitivity to contact inhibition, and may be less likely than normal cells to stop moving when surrounded by other cells; and they have lost their dependence on anchorage for cell growth, and may continue to divide in liquid or semisolid surroundings, whereas normal cells must be attached to a solid surface to grow.

The invention provides a method of treating cancer in a subject by providing a composition comprising an antibody specifically binding to and/or interfering with the biological activity of an isolated nucleic acid molecule comprising at least one polynucleotide sequence chosen from SEQ ID NOS.:1-6, and 31-38; sequences that hybridize to these sequences of under high stringency conditions; sequences having at least 80% sequence identity to the sequences of SEQ ID NOS.: 1-6, and 31-38 or sequences that hybridize to them under high stringency conditions; complements of any of these sequences; or biologically active fragments of any of the above-listed sequences; or an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, and 31-38, or a biologically active fragment thereof; or a fragment of such an antibody; and a pharmaceutically acceptable carrier or excipient; and administering the antibody composition to the subject.

The invention also provides a method of treating kidney cancer, cervical cancer, squamous lung cancer, ovarian cancer, bladder cancer, breast cancer, endometrial cancer, prostate cancer, and/or in a subject by providing a composition comprising an antibody specifically binding to and/or interfering with the biological activity of an isolated nucleic acid molecule comprising at least one polynucleotide sequence chosen from SEQ ID NOS.:1-6, and 31-38; sequences that hybridize to these sequences of under high stringency conditions; sequences having at least 80% sequence identity to the sequences of SEQ ID NOS.:1-6, and 31-38 or sequences that hybridize to them under high stringency conditions; complements of any of these sequences; or biologically active fragments of any of the above-listed sequences; or an isolated polypeptide comprising an amino acid sequence, wherein the amino acid sequence is chosen from SEQ ID NOS.:7-30, or a biologically active fragment thereof, or is encoded by a polynucleotide sequence chosen from SEQ ID NOS.:1-6, and 31-38, or a biologically active fragment thereof; or a fragment of such an antibody; and a pharmaceutically acceptable carrier or excipient, wherein the antibody specifically binds to or interferes with the activity of a polypeptide chosen from SEQ ID NOS.:7-30, or an active fragment thereof; and administering an amount of the antibody composition to the subject effective to treat kidney cancer, cervical cancer, squamous lung cancer, ovarian cancer, bladder cancer, breast cancer, endometrial cancer, prostate cancer, or skin cancer, respectively.

Treatment herein refers to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. That is, “treatment” includes (1) preventing the disorder from occurring or recurring in a subject who may be predisposed to the disorder but has not yet been diagnosed as having it, (2) inhibiting the disorder, such as arresting its development, (3) slopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, and/or tumor size.

The polynucleotides, polypeptides, and antibodies described above can be used to treat cancer. In an embodiment, a fusion protein or conjugate can additionally comprise a tumor-targeting moiety. Suitable moieties include those that enhance delivery of an therapeutic molecule to a tumor. For example, compounds that selectively bind to cancer cells compared to normal cells, selectively bind to tumor vasculature, selectively bind to the tumor type undergoing treatment, or enhance penetration into a solid tumor are included in the invention. Tumor targeting moieties of the invention can be peptides. Nucleic acid and amino acid molecules of the invention can be used alone or as an adjunct to cancer treatment. For example, a nucleic acid or amino acid molecules of the invention may be added to a standard chemotherapy regimen. It may be combined with one or more of the wide variety of drugs that have been employed in cancer treatment, including, but are not limited to, cisplatin, taxol, etoposide, Novantrone (mitoxantrone), actinomycin D, camptohecin (or water soluble derivatives thereof), methotrexate, mitomycins (e.g., mitomycin C), dacarbazine (DTIC), and anti-neoplastic antibiotics such as doxorubicin and daunomycin.

Drugs employed in cancer therapy may have a cytotoxic or cytostatic effect on cancer cells, or may reduce proliferation of the malignant cells. Drugs employed in cancer treatment can also be peptides. A nucleic acid or amino acid molecules of the invention can be combined with radiation therapy. A nucleic acid or amino acid molecules of the invention may be used adjunctively with therapeutic approaches described in De Vita et al., 2001. For those combinations in which a nucleic acid or amino acid molecule of the invention and a second anti-cancer agent exert a synergistic effect against cancer cells, the dosage of the second agent may be reduced, compared to the standard dosage of the second agent when administered alone. A method for increasing the sensitivity of cancer cells comprises co-administering a nucleic acid or amino acid molecule of the invention with an amount of a chemotherapeutic anti-cancer drug that is effective in enhancing sensitivity of cancer cells. Co-administration may be simultaneous or non-simultaneous administration. A nucleic acid or amino acid molecule of the invention may be administered along with other therapeutic agents, during the course of a treatment regimen. In one embodiment, administration of a nucleic acid or amino acid molecule of the invention and other therapeutic agents is sequential. An appropriate time course may be chosen by the physician, according to such factors as the nature of a patient's illness, and the patient's condition.

The invention also provides a method for prophylactic or therapeutic treatment of a subject needing or desiring such treatment by providing a vaccine, that can be administered to the subject. The vaccine may comprise one or more of a polynucleotide, polypeptide, or modulator of the invention, for example an antibody vaccine composition, a polypeptide vaccine composition, or a polynucleotide vaccine composition, useful for treating cancer, proliferative, inflammatory, immune, metabolic, bacterial, or viral disorders.

For example, the vaccine can be a cancer vaccine, and the polypeptide can concomitantly be a cancer antigen. The vaccine may be an anti-inflammatory vaccine, and the polypeptide can concomitantly be an inflammation-related antigen. The vaccine may be a viral vaccine, and the polypeptide can concomitantly be a viral antigen. In some embodiments, the vaccine comprises a polypeptide fragment, comprising at least one extracellular fragment of a polypeptide of the invention, and/or at least one extracellular fragment of a polypeptide of the invention minus the signal peptide, for the treatment, for example, of proliferative disorders, such as cancer. In certain embodiments, the vaccine comprises a polynucleotide encoding one or more such fragments, administered for the treatment, for example, of proliferative disorders, such as cancer. Further, the vaccine can be administered with or without an adjuvant.

Vaccine therapy involves the use of polynucleotides, polypeptides, or agents of the invention as immunogens for tumor antigens (Machiels et al., 2002). For example, peptide-based vaccines of the invention include unmodified subject polypeptides, fragments thereof, and MHC class I and class II-restricted peptide (Knutson et al., 2001), comprising, for example, the disclosed sequences with universal, nonspecific MHC class II-restricted epitopes. Peptide-based vaccines comprising a tumor antigen can be given directly, either alone or in conjunction with other molecules. The vaccines can also be delivered orally by producing the antigens in transgenic plants that can be subsequently ingested (U.S. Pat. No. 6,395,964).

In some embodiments, antibodies themselves can be used as antigens in anti-idiotype vaccines. That is, administering an antibody to a tumor antigen stimulates B cells to make antibodies to that antibody, which in turn recognize the tumor cells

Nucleic acid-based vaccines can deliver tumor antigens as polynucleotide constructs encoding the antigen. Vaccines comprising genetic material, such as DNA or RNA, can be given directly, either alone or in conjunction with other molecules. Administration of a vaccine expressing a molecule of the invention, e.g., as plasmid DNA, leads to persistent expression and release of the therapeutic immunogen over a period of time, helping to control unwanted tumor growth.

In some embodiments, nucleic acid-based vaccines encode subject antibodies. In such embodiments, the vaccines (e.g., DNA vaccines) can include post-transcriptional regulatory elements, such as the post-transcriptional regulatory acting RNA element (WPRE) derived from Woodchuck Hepatitis Virus. These post-transcriptional regulatory elements can be used to target the antibody, or a fusion protein comprising the antibody and a co-stimulatory molecule, to the tumor microenvironment (Pertl et al., 2003).

Besides stimulating anti-tumor immune responses by inducing humoral responses, vaccines of the invention can also induce cellular responses, including stimulating T-cells that recognize and kill tumor cells directly. For example, nucleotide-based vaccines of the invention encoding tumor antigens can be used to activate the CD8⁺ cytotoxic T lymphocyte arm of the immune system.

In some embodiments, the vaccines activate T-cells directly, and in others they enlist antigen-presenting cells to activate T-cells. Killer T-cells are primed, in part, by interacting with antigen-presenting cells, i.e., dendritic cells. In some embodiments, plasmids comprising the nucleic acid molecules of the invention enter antigen-presenting cells, which in turn display the encoded tumor-antigens that contribute to killer T-cell activation. Again, the tumor antigens can be delivered as plasmid DNA constructs, either alone or with other molecules.

In further embodiments, RNA can be used. For example, dendritic cells can be transfected with RNA encoding tumor antigens (Heiser et al., 2002; Mitchell and Nair, 2000). This approach overcomes the limitations of obtaining sufficient quantities of tumor material, extending therapy to patients otherwise excluded from clinical trials. For example, a subject RNA molecule isolated from tumors can be amplified using RT-PCR. In some embodiments, the RNA molecule of the invention is directly isolated from tumors and transfected into dendritic cells with no intervening cloning steps.

In some embodiments the molecules of the invention are altered such that the peptide antigens are more highly antigenic than in their native state. These embodiments address the need in the art to overcome the poor in vivo immunogenicity of most tumor antigens by enhancing tumor antigen immunogenicity via modification of epitope sequences (Yu and Restifo, 2002).

Another recognized problem of cancer vaccines is the presence of preexisting neutralizing antibodies. Some embodiments of the present invention overcome this problem by using viral vectors from non-mammalian natural hosts, i.e., avian pox viruses. Alternative embodiments that also circumvent preexisting neutralizing antibodies include genetically engineered influenza viruses, and the use of “naked” plasmid DNA vaccines that contain DNA with no associated protein. (Yu and Restifo, 2002).

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines.

For example, in some embodiments, nucleic acid vaccines encode an alphaviral replicase enzyme, in addition to tumor antigens. This recently discovered approach to vaccine therapy successfully combines therapeutic antigen production with the induction of the apoptotic death of the tumor cell (Yu and Restifo, 2002).

In some embodiments, a protein of the present invention is involved in the control of cell proliferation, and an agent of the invention inhibits undesirable cell proliferation. Such agents are useful for treating disorders that involve abnormal cell proliferation, including, but not limited to, cancer, psoriasis, and scleroderma. Whether a particular agent and/or therapeutic regimen of the invention is effective in reducing unwanted cellular proliferation, e.g., in the context of treating cancer, can be determined using standard methods. For example, the number of cancer cells in a biological sample (e.g., blood, a biopsy sample, and the like), can be determined. The tumor mass can be determined using standard radiological or biochemical methods.

The polynucleotides, polypeptides, and modulators of the present invention find use in immunotherapy of hyperproliferative disorders, including cancer, neoplastic, and paraneoplastic disorders. That is, the subject molecules can correspond to tumor antigens, of which 1770 have been identified to date (Yu and Restifo, 2002). Immunotherapeutic approaches include passive immunotherapy and vaccine therapy and can accomplish both generic and antigen-specific cancer immunotherapy.

Passive immunity approaches involve antibodies of the invention that are directed toward specific tumor-associated antigens. Such antibodies can eradicate systemic tumors at multiple sites, without eradicating normal cells. In some embodiments, the antibodies are combined with radioactive components, as provided above, for example, combining the antibody's ability to specifically target tumors with the added lethality of the radioisotope to the tumor DNA.

Useful antibodies comprise a discrete epitope or a combination of nested epitopes, i.e., a 10-mer epitope and associated peptide multimers incorporating all potential 8-mers and 9-mers, or overlapping epitopes (Dutoit et al., 2002). Thus a single antibody can interact with one or more epitopes. Further, the antibody can be used alone or in combination with different antibodies, that all recognize either a single or multiple epitopes.

Neutralizing antibodies can provide therapy for cancer and proliferative disorders. Neutralizing antibodies that specifically recognize a protein or peptide of the invention can bind to the protein or peptide, e.g., in a bodily fluid or the extracellular space, thereby modulating the biological activity of the protein or peptide. For example, neutralizing antibodies specific for proteins or peptides that play a role in stimulating the growth of cancer cells can be useful in modulating the growth of cancer cells. Similarly, neutralizing antibodies specific for proteins or peptides that play a role in the differentiation of cancer cells can be useful in modulating the differentiation of cancer cells.

Apoptosis, or programmed cell death, is a regulated process leading to cell death via a series of well-defined morphological changes. Programmed cell death provides a balance for cell growth and multiplication, eliminating unnecessary cells. The default state of the cell is to remain alive. A cell enters the apoptotic pathway when an essential factor is removed from the extracellular environment or when an internal signal is activated. Genes and proteins of the invention that suppress the growth of tumors by activating cell death provide the basis for treatment strategies for hyperproliferative disorders and conditions.

Inflammation and Immunity

In other embodiments, e.g., where the subject polypeptide is involved in modulating inflammation or immune function, the invention provides agents for treating such inflammation or immune disorders. For example, neutralizing antibodies can provide immunosuppressive therapy for inflammatory and autoimmune disorders. Neutralizing antibodies can be used to treat disorders such as, for example, multiple sclerosis, rheumatoid arthritis, inflammatory bowel disease, transplant rejection, and psoriasis. Neutralizing antibodies that specifically recognize a protein or peptide of the invention can bind to the protein or peptide, e.g., in a bodily fluid or the extracellular space, thereby modulating the biological activity of the protein or peptide. For example, neutralizing antibodies specific for proteins or peptides that play a role in activating immune cells are useful as immunosuppressants.

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the s appended hereto.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended s. Moreover, advantages described in the body of the specification, if not included in the s, are not per se limitations to the ed invention.

It is to be understood that both the foregoing general description and the following detailed description are exemplary and explanatory only and are not restrictive of the invention, as ed. Moreover, it must be understood that the invention is not limited to the particular embodiments described, as such may, of course, vary. Further, the terminology used to describe particular embodiments is not intended to be limiting, since the scope of the present invention will be limited only by its s.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended s, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and s, are modified by the term “about,” unless otherwise indicated. Accordingly, the numerical parameters set forth in the specification and s are approximations that may vary depending upon the desired properties of the present invention. At the very least, and not as an attempt to limit the application of the doctrine of equivalents to the scope of the s, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Expression of Probes to KIAA0779

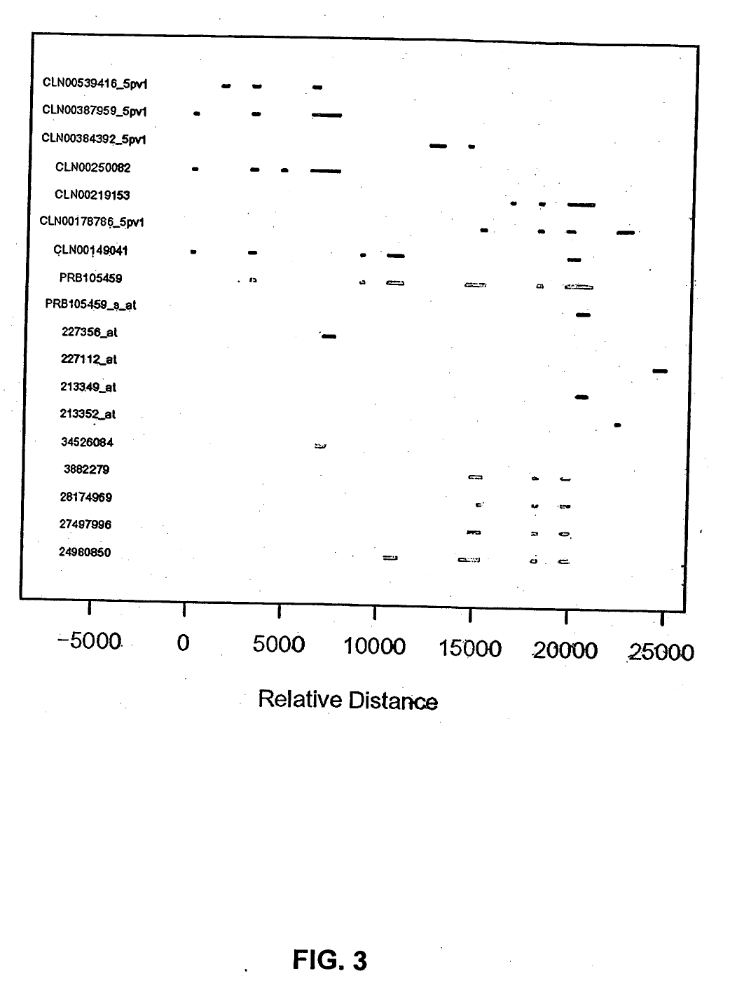

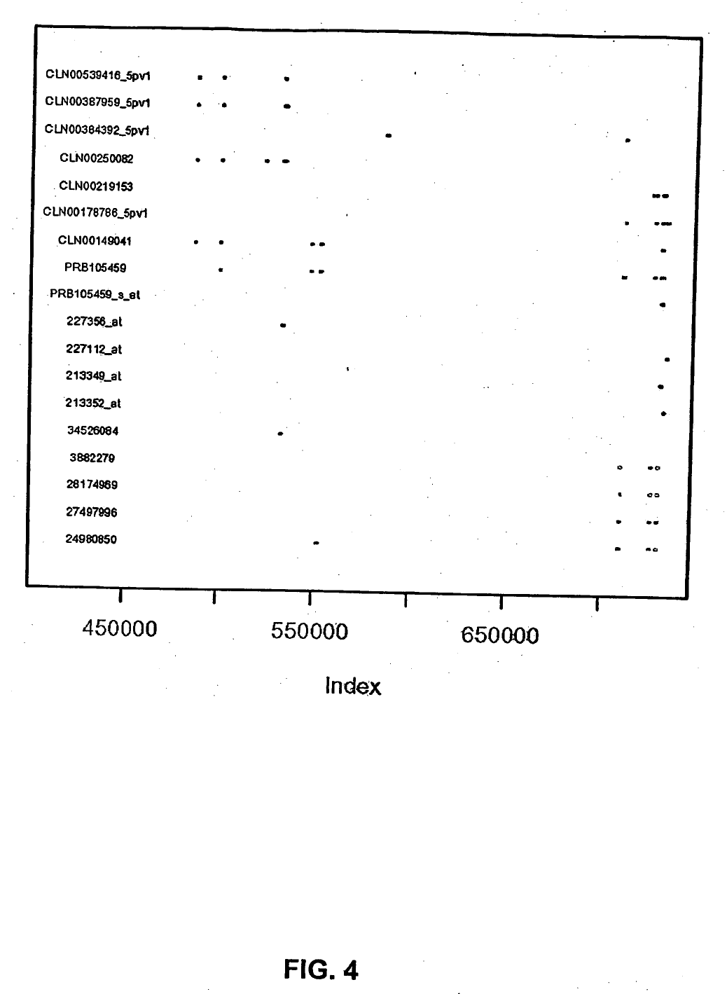

The GeneLogic oncology database, which was constructed on the basis of expression profiles of probe sets from the Affymetrix U133 microarray chip in normal and cancer tissues, was interrogated for transmembrane proteins that were overexpressed in different tumor types. As illustrated in the exon map shown in FIG. 3, the probe sequences Affy 213349_at, is herein found to map to the KIAA0779 protein sequence (24980850). This sequence is overexpressed in malignant kidney cancer compared to selected normal tissues.

The identification of Affy 213349_at as mapping to the KIAA0779 protein sequence 24980850 was made by mapping all publicly known cDNA sequences, gene sequences, and ESTs to the human genome map and creating a clustering system. Sequences that map to a particular locus of the human genome are designated as belonging to a corresponding cluster. By this method, splice variants of a gene family map to the same cluster. This clustering, gene mapping, and sequencing of full-length cDNA clones provides a basis for determining the expression patterns of KIAA0779 clones.

Additionally, human cDNA libraries were constructed from a number of human tissues. Clones from these libraries were categorized on the basis of the clustering system. The publicly disclosed KIAA0779 protein and the Affy probe 213349 mapped to the same cluster. Several novel splice variants also map to this cluster. These splice variants were sequenced and exon maps of these clones were produced, as shown in FIG. 3.

Novel clone CLN00149041 is homologous to probe PRB105459, which comprises untranslated regions (UTR) of clone 24980850. Clone CLN00149041 is a splice variant of the public clone and is missing the two transmembrane domains of the public clone. The N-termini of CLN001491041 and 24980850 clustered with the novel clones CLN00539416, CLN00387959, and CLN00250082. The C-termini of CLN00149041 and 24980850 clustered with the novel clone CLN00219153, and the previously disclosed clone CLN00178786.

Affy probe 213352 mapped to clone CLN00178786. Unlike Affy probe 213349, which is highly expressed in kidney cancer tissues as compared to tissues of selected normal organs, the 213352 probe did not generate a satisfactory expression profile for use as a target for antibody production for treatment of cancer. Other probes were observed to detect antisense sequences and intronic regions of the KIAA0779 gene, but were not useful for determining KIAA0779 expression patterns.

CLN00387959 and CLN00539416 have the same protein translation, although each has different UTR sequences. The sequence of Affy probe 227356 was mapped to CLN00539416, CLN00387959, and CLN00250082. This probe was found to be highly expressed in a number of tumor tissues and is useful as an antibody target, as shown in Tables 4 and 5.

Affy probe 213349 and the herein disclosed probe PRB105459_s_at, which was constructed on the basis of RPB105459, mapped to CLN00219153. This probe was found to be highly expressed in kidney cancer and also has a desirable expression profile for use as an antibody target, as shown in Tables 6 and 7.

### REFERENCES

The specification is most thoroughly understood in light of the following references, all of which are hereby incorporated by reference in their entireties. The disclosures of the patents and other references cited above are also hereby incorporated by reference.
1. Alberts, B., et al., (1994) Molecular Biology of the Cell. 3^{rd} ed. Garland Publishing, Inc.
2. Andreeff, M., Pinkel, D. eds. (1999) Introduction to Fluorescence In Situ Hybridization: Principles and Clinical Applications. John Wiley & Sons.
3. Ballance, D. J., et al. (1983) Biochem. Biophys. Res. Commun. 112:284-289.
4. Barany, F. (1985) Gene 37:111-123.
5. Barnes, D., Sato, G. (1980) Anal. Biochem. 102:255-270.
6. Barton, M. C., et al. (1990) Nucleic Acids Res. 18:7349-7355.
7. Bashkin, J. K., et al. (1995) Appl. Biochem. Biotechnol. 54:43-56.
8. Bassett, D. E., et al. (1999) Nature Genetics 21:51-55.
9. Battini, R., et al. (1987). J. Biol. Chem. 262:4355-4359.
10. Beach, D., et al. (1982) Nature 300:706-709.
11. Beigelman, L., et al. (1995) Nucleic Acids Res. 23:4434-4442.
12. Blackwell, J. M., et al. (1995) Mol. Med. 1:194-205.
13. Boshart, M., et al. (1985) Cell 41:521-530.
14. Bowtell, D. D. L. (1999) Nature Genetics 21:25-32.
15. Brenner, S., et al. (2000) Proc. Natl. Acad. Sci. USA 97:1665-1670.
16. Brown, P. O, Botstein, D. (1999) Nature Genetics 21:33-37.
17. Campbell, K. H., et al. (1996) Nature 380:64-66.
18. Carbonell, L. F., et al., (1988) Gene 73:409-418.
19. Carver, A. S., et al. (1993) Biotechnology (N.Y.) 11:1263-1270.
20. Chalifour, L. E., et al. (1994) Anal. Biochem. 216: 299-304.
21. Chang, A. C., et al. (1978) Nature 275:617-624.
22. Chen, S. Y., et al. (1994) Hum. Gene Ther. 5:595-601.
23. Cheng, W. F., et al. (2001) J. Clin. Invest. 108:669-678.
24. Cheung, V. G., et al. (1999) Nature Genetics 21:15-19.
25. Chien, C., et al. (1991) Proc. Nail. Acad. Sci. 88:9578-9581.
26. Cole, K. A., et al. (1999) Nature Genetics 21:3841.
27. Colicelli, J., et al. (1985) Mol. Gen. Genet. 199:537-539.
28. Collins, F. S. (1999) Nature Genetics 21:2.
29. Cregg, J. M., et al., (1985) Mol. Cell. Biol. 5:3376-3385.
30. Das, S., et al. (1984) J. Bacteriol. 158:1165-1167.
31. Davidow, L. S. et al. (1987) Curr. Genet. 11:377-383.
32. De Louvencourt, L., et al. (1983) J. Bacteriol. 154:737-742.
33. Delahunty, C., et al. (1996) Am. J. Human Genetics 58:1239-12, 46.
34. Deutscher, M. P., et al., eds. (1990) Guide to Protein Purification: Methods in Enzymology. (Methods in Enzymology Series, Vol 182). Academic Press.
35. Dieffenbach, C. W., Dveksler, G. S., eds. (1995) PCR Primer: A Laboratory Manual. Cold Spring Harbor Laboratory Press.
36. Dijkema, R., et al. (1985) EMBO J. 4:761-767.
37. Doerfler, W., Bohm, P., eds. (1987) The Molecular Biology Of Baculoviruses. Springer-Verlag, Inc.
38. Dutoit, V., et al. (2002) J. Clin. Invest. 110:1813-1822.
39. Espejo, A., et al. (2002) Biochem. J. 367:697-702.
40. Fields, S., Song, O. (1989) Nature 340:245-246.
41. Fisch, P., et al. (1993) Oncogene 8:3271-3276.
42. Gaillardin, C., Ribet, A. M. (1987) Curr. Genet. 11:369-375.
43. Gao, X., Nawaz, Z. (2002) Breast Cancer Res. 4:182-186.
44. Gaudilliere, B., et al. (2002) J. Biol. Chem. 277:46,442-46,446.
45. Geffen D. B., Man S. (2002) Isr. Med. Assoc. J. 4:1124-31.
46. Gennaro, A. R. (2003) Remington: The Science and Practice of Pharmacy with Facts and Comparisons: DrugfactsPlus. 20th ed., Lippincott Williams & Williams.
47. Gingras, M. C., et al. (2002) Mol. Immunol. 38:817-824.
48. Goeddel, D. V., et al. (1979) Nature 281:544-548.
49. Gonnet, G. H., et al. (1992) Science 256:1443-1445.
50. Gordon, J. W. (1989) Int. Rev. Cytol. 115:171-229.
51. Gorman, C. M., et al. (1982) Proc. Natl. Acad. Sci. 79:6777-6781.
52. Griffiths, M., et al. (1997) Nat. Med. 3:89-93.
53. Grosschedl, R., Baltimore, D. (1985) Cell 41:885-897.
54. Grosveld, F., Kollias, G., eds. (1992) Transgenic Animals. 1^{st} ed. Academic Press.
55. Gu, H., et al. (1994) Science. 265:103-106.
56. Gustin, K., Burk, R. D. (1993) Biotechniques 14:22-24.
57. Hacia, J. G. (1999) Nature Genetics 21:42-47.
58. Ham, R. G., McKeehan, W. L. (1979) 58:44-93.
59. Harlow, E., et al., eds. (1998) Using Antibodies: A Laboratory Manual: Portable Protocol NO. 1. Cold Spring Harbor Laboratory.
60. Harlow, E., Lane, D., eds. (1988) Antibodies: A Laboratory Manual. Cold Spring Harbor Laboratory.
61. Hassanzadeh, G. H. G., et al., (1998) FEBS Lett. 437:75-80.
62. Heiser, A., et al. (2002) J. Clin. Invest. 109:409-417.
63. Hinnen, A., et al., (1978) Proc. Natl. Acad. Sci. 75:1929-1933.
64. Hong, G. F. (1982) Biosci. Rep. 2:907-912.
65. Hoogenboom, H. R., et al. (1998) Immunotechnology 4:1-20.
66. Hooper, M. L. (1993) Embryonal Stem Cells: Introducing Planned Changes into the Animal Germline. Gordon & Breach Science Pub.
67. Houseman, B. T., et al. (2002) Nature Biotechnol. 20:270-274.
68. Howard, G. C., Bethell, D. R. (2000) Basic Methods in Antibody Production and Characterization. CRC Press.
69. Ito, H., et al. (1978) J. Bacteriol. 153:163-168.
70. Jameson, D. M., Sawyer, W. H. (1995) Methods Enzymol. 246:283-300.
71. Janeway, C. A., et al., (2001) Immunobiology. 5^{th} ed. Garland Publishing.
72. Jones, D. H., Winistorfer, S. C. (1992) Biotechniques 12:528-530.
73. Jones, P., ed. (1998a) Vectors: Cloning Applications: Essential Techniques, John Wiley & Son, Ltd.
74. Jones, P., ed. (1998b) Vectors: Expression Systems: Essential Techniques, John Wiley & Son, Ltd.
75. Jost, C. R., et al. (1994) J. Biol. Chem. 269:26,267-26,273.
76. Joulin, V., Richard-Foy, H. (1995) Eur. J. Biochem. 232:620-626.
77. Kabat, E. A., Wu T. T. (1991) J. Immunol. 147:1709-1719.
78. Kari, C., et al. (2003) Cancer Res. 63:1-5.
79. Kelly, J. M., Hynes, M. J. (1985) EMBO J. 4:475-479.
80. Keown, W. A., et al. (1990) Methods Enzymol. 185:527-537.
81. Kibbe, A. H., ed. (2000) Handbook of Pharmaceutical Excipients. 3^{rd} ed. Pharmaceutical Press.
82. Knutson, K. L., et al. (2001) J. Clin. Invest. 107:477-484.
83. Kolonin, M. G., et al. (1998) Proc. Natl. Acad. Sci. 95:14,266-14,271.
84. Ku, D. H., et al. (1990) J. Biol. Chem. 265:16,060-16,063.
85. Kunze, G. et al. (1985) J. Basic Microbiol. 25:141-144.
86. Kurtz, M. B., et al. (1986) Mol. Cell. Biol. 6:142-149.
87. Lakso, M., et al. (1992) Proc. Natl. Acad. Sci. 89:6232-6236
88. Lander, E. S. (1999) Array of hope. Nature Genetics 21:34.
89. Lander, E. S., et al. (2001) Nature 409:860-921.
90. Lavitrano, M., et al. (1989) Cell 57:717-723.
91. Lebacq-Verheyden, A. M., et al. (1988) Mol. Cell. Biol. 8:3129-3135.
92. Lerner, R. A. (1982) Nature 299:592-596.
93. Li, E., et al. (1996), Eur. J. Biochem. 238:631-638.
94. Lipshutz, R. J., et al. (1999) Nature Genetics 21:20-24.
95. Liu A. Y., et al. (1987a) Proc. Natl. Acad. Sci. 84:3439-3443.
96. Liu, A. Y., et al. (1987b) J. Immunol. 139:3521-3526.
97. Lodish, H., et al., (1999) Molecular Cell Biology. 4th ed. W H Freeman & Co.
98. Luckow, V., Summers, M. (1988) Biotechnology 6:47-55.
99. MacBeath, G., Schreiber. S. L. (2000) Science 289:1760-1763.
100. Machiels, J. P., et al., (2002) Semin. Oncol. 29:494-502.
101. Mackay, A., et al., (2003) Oncogene 22:2680-2688.
102. Maeda, S., et al. (1985) Nature 315:592-594.
103. Marotti, K. R., Tomich, C. S. (1989) Gene Anal. Tech. 6:67-70.
104. Martin, B. M., et al. (1988) DNA 7:99-106.
105. Matz, M. V., et al., (1999) Nat. Biotechnol. 17:969-973.
106. McGraw, R. A. III (1984) Anal. Biochem. 143:298-303.
107. McPherson, M. J., et al., (2000) PCR Basics: From Background to Bench. Springer Verlag.
108. Milligan, J. F., et al., (1993) J. Med. Chem. 36:1923-1937.
109. Mitchell, D. A., Nair, S. K. (2000) J. Clin. Invest. 106:1065-1069.
110. Miyajima, A., et al. (1987) Gene 58:273-281.
111. Monfardini, C., et al., (1995) Bioconjugate Chem. 6, 62-69.
112. Mortlock, D. P., et al., (1996) Genome Res. 6:327-335.
113. Murphy, D., Carter, D. A., eds. (1993) Transgenesis Techniques: Principles and Protocols. Humana Press.
114. Okayama, H., Berg, P. (1983) Mol. Cell. Biol. 3:280-289.
115. O'Neil, N. J., et al., (2001) Am. J. Pharmacogenomics 1:45-53.
116. O'Neill, L. A. (2002) Curr. Top. Microbiol. Immunol. 270:47-61.
117. Pan, C. X., Koeneman, K. S. (1999) Med. Hypothesis 53:130-135.
118. Paterson, T., et al., (1994) Appl. Microbiol. Biotechnol. 40:691-698.
119. Acad. Sci. 89:10,974-10,978.
120. Peelle, B., et al., (2001) J. Protein Chem. 20:507-519.
121. Pertl, U., et al., (2003) Blood 101:649-654.
122. Phillips, M. I., ed. (1999a) Antisense Technology, Part A, Methods in Enzymology Vol. 313. Academic Press, Inc.
123. Phillips, M. I., ed. (1999b) Antisense Technology, Part B, Methods in Enzymology Vol. 314. Academic Press, Inc.
124. Pietu, G., et al. (1996) Genome Res. 6:492-503.
125. Pinkert, C. A., ed. (9994) Transgenic Animal Technology: A Laboratory Handbook. Academic Press.
126. Prentki, P., Krisch, H. M. (1984) Gene 29:303-313.
127. Ramalho-Santos, M. (2002) Science 298:597-6600.
128. Raval, P. (1994) J. Pharmacol. Toxicol. Methods 32:125-127.
129. Riley, J., et al. (1990) Nuc. Acids Res. 18:2887-2890.
130. Roggenkamp, R., et al. (1984) Mol. Gen. Genet. 194:489-493.
131. Saiki, R. K, et al. (1988) Science 239:487-491.
132. Sambrook, J., Russell, D. W., Sambrook, J. (1989) Molecular Cloning, A Laboratory Manual. 2^{nd} ed. Cold Spring Harbor Laboratory Press.
133. Sayers, J. R., et l. (1992) Biotechniques 13:592-596.
134. Schaeferling, M., et al. (2002) Electrophoresis 23:3097-3105.
135. Schaffer, J. E., Lodish, H. F. (1994) Cell 79:393-395.
136. Schena, M., ed. (1999) DNA Microarrays: A Practical Approach. Oxford Univ. Press.
137. Schena, M., ed. (2000) Microarray Biochip Technology. 1st ed. Eaton Publishing Co.
138. Setlow, J., Hollaender, A., eds. (1986) Genetic Engineering: Principles and Methods. Plenum Pub. Corp.
139. Shinnick, T. M., et al. (1983) Ann. Rev. Microbiol. 37:425-446.
140. Siebenlist, U., et al. (1980) Cell 20:269-281.
141. Smith, G. E., et al (1985) Proc. Natl. Acad. Sci. 82:8404-8408.
142. Soares, M. B. (1997) Curr. Opin. Biotechnol. 8:542-546.
143. Southern, E., et al. (1999) Nature Genetics 21:5-9.
144. Steinhaur, C., et al. (2002) Biotechniques, Supp. :38-45.
145. Stolz, L. E., Tuan, R. S. (1996) Mol. Biotechnol. 6:225-230.
146. Sutcliffe, J. G., et al (1983) Science 219:660-1666.
147. Thompson, S. et al. (1989) Cell 56:313-321.
148. Tilburn, J., et al. (1983) Gene 26:205-221.
149. Ulmer, J. B., et al., (1993) Science 259:1745-1749.
150. Van Den Blink, B., et al. (2002) Ann. N.Y. Acad. Sci. 973:349-58.
151. van der Putten, H., et al. (1985) Proc. Natl. Acad. Sci. 82:6148-6152.
152. Vlak, J. M., et al. (1988) J. Gen. Virol. 69:765-776.
153. Voisset, C., et al. (2000) AIDS Res. Hum. Retroviruses 36:731-740.
154. Wagner, R. W., et al. (1996) Nat. Biotechnol. 14:840-844.
155. Wagner, R. W., et al. (1993) Science 260:151-1513.
156. Wei, S., et al. (1997) Immunogenetics 45:405-412.
157. Weiner, M. P., et al. (1993) Gene 126:35-41.
158. Weng, S., et al. (2002) Proteomics 2:48-57.
159. Winssinger, N., et al. (2002) Proc. Natl. Acad. Sci. 99:11,139-11,144.
160. Wright, G., et al. (1991) Biotechnology (N.Y.) 9:830-834.
161. Xu, C. W., et al. (1997) Proc. Natl. Acad. Sci. 94:12,473-12,478.
162. Yelton, M. M., et al. (1984) Proc. Natl. Acad. Sci. 81:1470-1474.
163. Yu, Z., Restifo, N. P. (2002) J. Clin. Invest. 110:289-294.
164. Zallipsky, S. (1995) Bioconjugate Chem., 6:150-165.
165. Zhao, N., et al. (1995) Gene 156:207-215.
166. Zhu, D. L. (1989) Anal. Biochem. 177:120-124.
167. Zhu, H., et al. (2000) Nat. Genetics 26:283-289.
168. Zhu, H., et al. (2001) Science 293:2101-2105.
169. Zhu, H., Snyder, M. (2003) Curr. Opin. Chem. Biol. 7:55-63.

170. Zhu, J., Kahn, C. R. (1997) Proc. Natl. Acad. Sci. 94:13,063-13,068.

TABLE 1
Identification of Novel KIAA0779 Clones and Antibody Targets

| | SEQ. ID. NO. | SEQ. ID. NO. | SEQ. ID. NO. | |
|---|---|---|---|---|
| FP ID | (N1) | (P1) | (N0) | Source ID |
| HG1018237 | SEQ. ID. NO. 1 | SEQ. ID. NO. 7 | SEQ. ID. NO. 31 | CLN00539416.a |
| HG1018238 | SEQ. ID. NO. 2 | SEQ. ID. NO. 8 | SEQ. ID. NO. 32 | CLN00250082.b |
| HG1018239 | SEQ. ID. NO. 3 | SEQ. ID. NO. 9 | SEQ. ID. NO. 33 | 24980850:24980849 |
| HG1018240 | | SEQ. ID. NO. 10 | | CLN00539416.a_nonTM_1 |
| HG1018241 | | SEQ. ID. NO. 11 | | CLN00539416.a_nonTM_2 |
| HG1018242 | | SEQ. ID. NO. 12 | | CLN00250082.b_nonTM_1 |
| HG1018243 | | SEQ. ID. NO. 13 | | CLN00250082.b_nonTM_2 |
| HG1018244 | | SEQ. ID. NO. 14 | | 24980850:24980849_nonTM_1 |
| HG1018245 | | SEQ. ID. NO. 15 | | 24980850:24980849_nonTM_2 |
| HG1018246 | | SEQ. ID. NO. 16 | | 24980850:24980849_nonTM_3 |
| HG1018247 | | | SEQ. ID. NO. 34 | 227356_at |
| HG1018248 | SEQ. ID. NO. 4 | SEQ. ID. NO. 17 | SEQ. ID. NO. 35 | CLN00219153.a |
| HG1018249 | SEQ. ID. NO. 5 | SEQ. ID. NO. 18 | SEQ. ID. NO. 36 | CLN00149041.a |
| HG1018250 | SEQ. ID. NO. 6 | SEQ. ID. NO. 19 | SEQ. ID. NO. 37 | 24980850:24980849 |
| HG1018251 | | SEQ. ID. NO. 20 | | CLN00219153.a_nonTM_1 |
| HG1018252 | | SEQ. ID. NO. 21 | | CLN00219153.a_nonTM_2 |
| HG1018253 | | SEQ. ID. NO. 22 | | CLN00219153.a_nonTM_3 |
| HG1018254 | | SEQ. ID. NO. 23 | | CLN00219153.a_altTM_1 |
| HG1018255 | | SEQ. ID. NO. 24 | | CLN00219153.a_altTM_2 |
| HG1018256 | | SEQ. ID. NO. 25 | | CLN00219153.a_altnonTM_1 |
| HG1018257 | | SEQ. ID. NO. 26 | | CLN00219153.a_altnonTM_2 |
| HG1018258 | | SEQ. ID. NO. 27 | | CLN00219153.a_altnonTM_3 |
| HG1018259 | | SEQ. ID. NO. 28 | | 24980850:24980849_nonTM_1 |
| HG1018260 | | SEQ. ID. NO. 29 | | 24980850:24980849_nonTM_2 |
| HG1018261 | | SEQ. ID. NO. 30 | | 24980850:24980849_nonTM_3 |
| HG1018262 | | | SEQ. ID. NO. 38 | 213349_at |

TABLE 2
Structural Characteristics of Group 1 Novel
KIAA0779 Clones and Antibody Targets

| | | Predicted | Mature | | | |
|---|---|---|---|---|---|---|
| | | Protein | Protein | | TM | non-TM |
| FP ID | Source ID | Length | Coords. | TM | Coords. | Coords. |
| HG1018237 | CLN00539416.a | 96 | (1-96) | 1 | (27-49) | (1-26) |
| | | | | | | (50-96) |
| HG1018238 | CLN00250082.b | 76 | (1-76) | 1 | (27-49) | (1-26) |
| | | | | | | (50-76) |
| HG1018239 | 24980850:24980849 | 653 | (1-653) | 2 | (592-614) | (1-591) |
| | | | | | (621-638) | (615-620) |
| | | | | | | (639-653) |

TABLE 3
Structural Characteristics of Group 2 Novel KIAA0779 Clones and Antibody Targets

| | | Pred. | Mat. | | | | | Alt. Non- |
|---|---|---|---|---|---|---|---|---|
| | | Protein | Prot. | | TM | Non-TM | Alt. TM | TM |
| FP ID | Source ID | Length | Coords. | TM | Coords. | Coords. | Coords. | Coords |
| HG1018248 | CLN00219153.a | 121 | (1-121) | 2 | (57-76) | (1-56) | (60-82) | (1-59) |
| | | | | | (91-113) | (77-90) | (89-106) | (83-88) |
| | | | | | | (114-121) | | (107-121) |
| HG1018249 | CLN00149041.a | 214 | (1-214) | 0 | | (1-214) | | |
| HG1018250 | 24980850:24980849 | 653 | (1-653) | 2 | (592-614) | (1-591) | | |
| | | | | | (621-638) | (615-620) | | |
| | | | | | | (639-653) | | |

TABLE 4
Expression of Probe 227356 in Normal Tissues

| | | | Relative | |
|---|---|---|---|---|
| | Number | % | Median | |
| Normal | of | Expres- | Level of | |
| Tissue | Samples | sion | Expression | Range |
| Adrenal | 14 | 85 | 200 | ND |
| Heart | 3 | 33 | 130 | ND |
| Bladder | 9 | 100 | 250 | 75^{th} percentile: 350; |
| | | | | maximum: 400 |
| Breast | 77 | 97 | 210 | maximum: 390 |
| Cervix | 105 | 91 | 210 | 75^{th} percentile: 380; |
| | | | | maximum: 440 |
| Endometrium | 15 | 100 | 260 | 75^{th} percentile: 650 |
| Ovary | 73 | 85 | 150 | 75^{th} percentile: 250; |
| | | | | maximum: 310 |
| Colon | 228 | 99 | 260 | ND |
| Kidney | 99 | 97 | 225 | ND |
| Liver | 48 | 86 | 210 | ND |
| Lung | 124 | 94 | 240 | 75^{th} percentile: 410; |
| | | | | maximum: 725 |
| Pancreas | 53 | 100 | 350 | ND |
| Prostate | 61 | 98 | 350 | 75^{th} percentile: 730 |
| Rectum | 48 | 100 | 275 | ND |
| Skin | 68 | 84 | 175 | ND |
| Stomach | 66 | 100 | 225 | ND |

TABLE 5
Expression of Probe 227356 in Cancer

| | | | Relative | |
|---|---|---|---|---|
| | Number | % | Median | |
| Malignant | of | Expres- | Level of | |
| Tissue | Samples | sion | Expression | Range |
| Bladder | 23 | 91 | 300 | 75^{th} percentile: 670; |
| | | | | maximum: 900 |
| Breast | 312 | 98 | 250 | 75^{th} percentile: 470; |
| | | | | maximum: 1200 |
| Cervix | 23 | 100 | 400 | 75^{th} percentile: 800; |
| | | | | maximum: 810 |
| Endometrium | 101 | 97 | 325 | 75^{th} percentile: 700; |
| | | | | maximum: 1100 |
| Lung | ND | 98 | 350 | 75^{th} percentile: 575; |
| (squamous) | | | | maximum: 1050 |
| Ovary | 130 | 99 | 300 | 75^{th} percentile: 650; |
| | | | | maximum: 900 |
| Prostate | 98 | 100 | 400 | 75^{th} percentile: 770; |
| | | | | maximum: 950 |
| Skin | 45 | 95 | 210 | 75^{th} percentile: 380; |
| | | | | maximum: 650 |
| Pancreas | 79 | 92 | 240 | ND |

TABLE 6
Expression of Probe 213349 in Normal Tissues

| | | | Relative | |
|---|---|---|---|---|
| | Number | % | Median | |
| Normal | of | Expres- | Level of | |
| Tissue | Samples | sion | Expression | Range |
| Adrenal | 14 | 85 | 200 | ND |
| Heart | 3 | 33 | 130 | ND |
| Bladder | 9 | 100 | 250 | 75^{th} percentile: 350 |
| Breast | 77 | 97 | 210 | maximum: 390 |
| Cervix | 105 | 91 | 210 | 75^{th} percentile: 380; |
| | | | | maximum: 440 |
| Endometrium | 15 | 100 | 260 | 75^{th} percentile: 650 |
| Ovary | 73 | 85 | 150 | 75^{th} percentile: 250; |
| | | | | maximum: 310 |
| Colon | 228 | 99 | 260 | ND |
| Kidney | 99 | 97 | 225 | ND |
| Liver | 48 | 86 | 210 | ND |
| Lung | 124 | 94 | 250 | 75^{th} percentile: 410; |
| | | | | maximum: 725 |
| Pancreas | 53 | 100 | 350 | ND |
| Prostate | 61 | 98 | 350 | 75^{th} percentile: 730 |
| Rectum | 48 | 100 | 275 | ND |
| Skin | 68 | 84 | 175 | ND |
| Stomach | 66 | 100 | 225 | ND |

TABLE 7
Gene Expression of Probe 213349 in Cancer

| | | | Relative | |
|---|---|---|---|---|
| | Number | % | Median | |
| Malignant | of | Expres- | Level of | |
| Tissue | Samples | sion | Expression | Range |
| Bladder | 23 | 91 | 300 | 75^{th} percentile: 670; |
| | | | | maximum: 900 |
| Breast | 312 | 98 | 250 | 75^{th} percentile: 470; |
| | | | | maximum: 1200 |
| Cervix | 23 | 100 | 400 | 75^{th} percentile: 800; |
| | | | | maximum: 810 |
| Endometrium | 101 | 97 | 325 | 75^{th} percentile: 700; |
| | | | | maximum: 1100 |
| Lung | | 98 | 350 | 75^{th} percentile: 575; |
| (squamous) | | | | maximum: 1050 |
| Ovary | 130 | 99 | 300 | 75^{th} percentile: 650; |
| | | | | maximum: 900 |
| Prostate | 98 | 100 | 400 | 75^{th} percentile: 770; |
| | | | | maximum: 950 |
| Skin | 45 | 95 | 210 | 75^{th} percentile: 380 |
| Pancreas | 79 | 92 | 240 | |

1. An isolated nucleic acid molecule comprising at least one polynucleotide sequence, wherein the polynucleotide sequence is chosen from
(a) SEQ ID NOS.: 1-2, 4-5, 31-32, and 35-36;
(b) sequences that hybridize to the sequences of (a) under high stringency conditions;
(c) sequences having at least 80% sequence identity to the sequences of (a) or (b);
(d) complements of the sequences of (a), (b) or c); and
(e) biologically active fragments of any of (a)-(d).

2. The nucleic acid molecule of claim 1, wherein the nucleic acid molecule is a DNA or a RNA molecule.

3. A double-stranded isolated nucleic acid molecule comprising the nucleic acid molecule of claim 1 and its complement.

4. The nucleic acid molecule of claim 1, wherein the polynucleotide sequence encodes a polypeptide or a biologically active fragment of the polypeptide.

5. A vector comprising the nucleic acid molecule of claim 1 and a promoter that regulates the expression of the nucleic acid molecule.

6. The vector of claim 5, wherein the promoter is chosen from one that is naturally contiguous to the nucleic acid molecule and a promoter that is not naturally contiguous to the nucleic acid molecule.

7. The vector of claim 5, wherein the promoter is a promoter chosen from an inducible promoter, a conditionally active promoter, a constitutive promoter, and a tissue specific promoter.

8. A recombinant host cell comprising the nucleic acid molecule of claim 1.

9. The host cell of claim 8, wherein the cell is a prokaryotic cell or a eukaryotic cell.

10. The host cell of claim 8, wherein the cell is a cell chosen from a human cell, a non-human mammalian cell, an insect cell, a fish cell, a plant cell, and a fungal cell.

11. An isolated polypeptide comprising an amino acid sequence, wherein the amino acid sequence is chosen from SEQ ID NOS.:7-8, 10-16, 17-18, 20-30, or a biologically active fragment thereof, or is encoded by a polynucleotide sequence c chosen from SEQ ID NOS.:1-2, 4-5, or a biologically active fragment thereof.

12. The polypeptide of claim 11, wherein the polypeptide comprises a non-transmembrane region.

13. The polypeptide of claim 12, wherein the polypeptide comprises an extracellular fragment.

14. A method of making a recombinant host cell comprising:
(a) providing a composition comprising the vector of claim 5, and
(b) allowing a host cell to come into contact with the vector to form a recombinant host cell.

15. A method of making a polypeptide comprising:
(a) providing a nucleic acid molecule encoding the polypeptide of claim 11;
(b) introducing the nucleic acid molecule into an expression system; and
(c) allowing the polypeptide to be produced.

16. A method of making a polypeptide comprising:
(a) providing a composition comprising the host cell of claim 8; and
(b) culturing the host cell to produce the polypeptide.

17. A method of determining the presence of the nucleic acid molecule of claim 1 in a sample comprising:
(a) providing a complement of the nucleic acid molecule or providing a complement to the complement of the nucleic acid molecule of claim 1;
(b) allowing the molecule to interact with the sample; and
(c) determining whether interaction has occurred.

18. A method of determining the presence of a specific antibody to the polypeptide of claim 11 in a sample, comprising:
(a) providing the polypeptide of claim 11;
(b) allowing the polypeptide to interact with a specific antibody in the sample, if present; and
(c) determining whether interaction has occurred.

19. A method of determining the presence of the polypeptide of claim 11 in a sample, comprising:
(a) providing an antibody that specifically binds to or interferes with the activity of the polypeptide;
(b) allowing the antibody to interact with the polypeptide in the sample, if any; and
(c) determining whether interaction has occurred.

20. An antibody specifically binding to and/or interfering, with the biological activity of the nucleic acid molecule of claim 1 or a biologically active fragment thereof, or the polypeptide of claim 11 or a biologically active fragment thereof.

21. The antibody of claim 20, wherein the antibody is chosen from a monoclonal antibody, a polyclonal antibody, a single chain antibody, an Fab fragment, an antibody comprising a backbone of a molecule with an Ig domain, a V_{H} fragment, a V_{L} fragment, a cdr fragment, and a framework fragment.

22. The antibody of claim 20, wherein the antibody is chosen from a cytotoxic antibody, targeting antibody, an antibody agonist, an antibody antagonist, an antibody that promotes endocytosis of a target antigen, an antibody that mediates ADCC, and an antibody that mediates CDC.

23. The antibody of claim 20, wherein the antibody is chosen from a human antibody, a non-human primate antibody, a non-primate animal antibody, a rabbit antibody, a mouse antibody, a rat antibody, a sheep antibody, a goat antibody, a horse antibody, a porcine antibody, a cow antibody, a chicken antibody, a humanized antibody, a primatized antibody, and a chimeric antibody.

24. The antibody of claim 23, wherein the antibody comprises a cytotoxic antibody that comprises one or more cytotoxic component chosen from a radioisotope, a microbial toxin, a plant toxin, and a chemical compound.

25. The cytotoxic antibody of claim 20, wherein the chemical compound is chosen from doxorubicin and cisplatin.

26. A composition comprising a pharmaceutically acceptable carrier or excipient and one or more active agents chosen from the nucleic acid molecule of claim 1, the vector of claim 5, the polypeptide of claim 11, and the antibody of claim 20.

27. An antibody directed to a polypeptide encoded by a nucleic acid molecule encoded by a nucleic acid molecule with SEQ ID NO.:1-6.

28. A bacteriophage comprising the antibody of claim 20 or a fragment thereof.

29. A bacterial cell comprising the bacteriophage of claim 27.

30. A recombinant host cell that produces the antibody of claim 20.

31. An animal injected with one or more active agents chosen from the nucleic acid molecule of claim 1, the vector of claim 5, the host cell of claim 8, the polypeptide of claim 11, and the antibody of claim 20.

32. A diagnostic kit comprising a nucleic acid molecule, wherein the nucleic acid molecule comprises at least 6 contiguous nucleotides from the nucleic acid molecule of claim 1.

33. A diagnostic kit comprising a polypeptide molecule, wherein the polypeptide molecule comprises an amino acid sequence, wherein the amino acid sequence is chosen from SEQ ID NOS.:7-30 or a biologically active fragment thereof, or is encoded by a polynucleotide sequence chosen from SEQ ID NOS.1-2, 4-5, and reagents to carry out an immunoassay.

34. A diagnostic kit comprising the antibody of claim 20 and reagents to carry out an immunoassay.

35. A method of making an antibody, comprising:
(a) introducing an antigen chosen from the nucleic acid molecule of claim 1 or the polypeptide of claim 11 into an animal in an amount sufficient to elicit generation of antibodies specific to the antigen, and
(b) recovering the antibodies therefrom.

36. A method of identifying a modulating agent that modulates the biological activity of the polypeptide of claim 11 comprising:
(a) providing the polypeptide of claim 11;
(b) allowing at least one modulating agent to contact the polypeptide; and
(c) selecting an agent that modulates the biological activity of the polypeptide.

37. A modulator composition comprising a modulator and a pharmaceutically acceptable carrier, wherein the modulator is obtainable by the method of claim 36.

38. The modulator composition of claim 37, wherein the modulator is an antibody or a small molecule drug.

39. A method of treating a disease, disorder, syndrome, or condition in a subject, comprising administering the composition of claim 26 to the subject.

40. The method of claim 39, wherein the disease, disorder, syndrome, or condition is proliferative, inflammatory, immune related, or metabolic.

41. The method of claim 39, wherein the disease is cancer.

42. A method of treating cancer in a subject, comprising:
(a) providing an antibody composition of claim 26; and
(b) administering the antibody composition to the subject.

43. A method of treating kidney cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat kidney cancer.

44. A method of treating cervical cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat cervical cancer.

45. A method of treating squamous lung cancer in a subject comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat squamous lung cancer.

46. A method of treating ovarian cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat ovarian cancer.

47. A method of treating bladder cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat bladder cancer.

48. A method of treating breast cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat breast cancer.

49. A method of treating endometrial cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat endometrial cancer.

50. A method of treating prostate cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat prostate cancer.

51. A method of treating skin cancer in a subject, comprising:
(a) providing the antibody composition of claim 26, wherein the antibody specifically binds to or interferes with the activity of a polypeptide chosen from SEQ ID NOS.:7-8, 10-18, 20-30, or an active fragment thereof; and
(b) administering an amount of the antibody composition to the subject effective to treat skin cancer.
